(19) United States
(12) Patent Application Publication
(10) Pub. No.: US 2006/0275870 A1
(43) Pub. Date: Dec. 7, 2006

(54) Novel G-protein coupled receptors and uses therefor

(75) Inventor: Wei Gu, Brookline, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/500,008

(22) Filed: Aug. 7, 2006

Related U.S. Application Data
(63) Continuation of application No. 10/664,667, filed on Sep. 18, 2003.
Application No. 10/664,667 is a continuation of application No. 09/851,595, filed on May 8, 2001, now abandoned.
Application No. 09/851,595 is a continuation-in-part of application No. 09/566,588, filed on May 8, 2000, now abandoned.
(60) Provisional application No. 60/132,896, filed on May 6, 1999.

(51) Int. Cl. G01N 33/567; C07H 21/04; C12P 21/06; C07K 14/705; C07K 16/28

(52) U.S. Cl. 435/69.1; 435/320.1; 435/325; 530/350; 536/23.5; 435/7.2; 530/388.22

(57) ABSTRACT

Novel G-protein coupled receptor molecules, designated LGR6 polypeptides, proteins, and nucleic acid molecules, are disclosed. In addition to isolated, LGR6 proteins, the invention further provides isolated LGR6 fusion proteins, antigenic peptides and anti-LGR6 antibodies. The invention also provides LGR6 nucleic acid molecules, recombinant expression vectors containing a nucleic acid molecule of the invention, host cells into which the expression vectors have been introduced and non-human transgenic animals in which an LGR6 gene has been introduced or disrupted. Diagnostic, screening and therapeutic methods utilizing compositions of the invention are also provided.

### RELATED APPLICATIONS

This application claims priority to U.S. patent application Ser. No. 09/566,588, filed May 8, 2000, which claims the benefit U.S. Provisional Application No. 60/132,896 filed on May 6, 1999, incorporated herein in their entirety by this reference.

### BACKGROUND OF THE INVENTION

G-protein coupled receptors (GPCRs) are seven transmembrane domain proteins that mediate signal transduction of a diverse number of ligands through heterotrimeric G proteins (Strader, C. D. et al. (1994) Annu. Rev Biochem. 63: 101-132). G protein-coupled receptors (GPCRs), along with G-proteins and effector proteins (e.g., intracellular enzymes and channels), are the components of a modular signaling system. Upon ligand binding to an extracellular portion of a GPCR, different G proteins are activated, which in turn modulate the activity of different intracellular effector enzymes and ion channels (Gutkind, J. S. (1998) J. Biol. Chem. 273: 1839-1842; Selbie, L. A. and Hill, S. J. (1998) Trends Pharmacol. Sci. 19:87-93).

G proteins represent a family of heterotrimeric proteins composed of α, β and γ subunits, which bind guanine nucleotides. These proteins are usually linked to cell surface receptors (e.g., GPCR). Following ligand binding to the GPCR, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits. Examples of mammalian G proteins include Gi, Go, Gq, Gs and Gt (Lodish H. et al. Molecular Cell Biology, (Scientific American Books Inc., New York, N.Y., 1995).

The GPCR protein superfamily identified to date contains over 250 subtypes. The superfamily can be broken down into five subfamilies: Subfamily I, which includes receptors typified by rhodopsin and the beta2-adrenergic receptor and currently contains over 200 unique members (reviewed by Dohlman et al. (1991) Annu. Rev. Biochem. 60:653-688); Subfamily II, which includes the parathyroid hormone/calcitonin/secretin receptor family (Juppner et al. (1991) Science 254:1024-1026; Lin et al. (1991) Science 254:1022-1024); Subfamily III, which includes the metabotropic glutamate receptor family in mammals, such as the GABA receptors (Nakanishi et al. (1992) Science 258: 597-603); Subfamily IV, which includes the cAMP receptor family that is known to mediate the chemotaxis and development of D. discoideum (Klein et al. (1988) Science 241:1467-1472); and Subfamily V, which includes the fungal mating pheromone receptors such as STE2 (reviewed by Kurjan I et al. (1992) Annu. Rev. Biochem. 61:1097-1129). Within each family, distinct, highly conserved motifs have been identified. These motifs have been suggested to be critical for the structural integrity of the receptor, as well as for coupling to G proteins.

Glycoprotein hormone receptors represent a subgroup of the Subfamily I of GPCRs. These hormone receptors have a large N-terminal extracellular (ecto-) domain which contains several leucine-rich repeats. The ligands for these receptors are glycoprotein hormones such as gonadotropins (e.g., lutenizing hormone (LH), follicle stimulating hormone (FSH), choriogonadotropin (CG) and thyrotropin (TSH)). Gonadotropins and TSH are essential for the growth and differentation of gonads and the thryoid glands, respectively. Binding of a glycoprotein hormone to these receptors leads to activation of the Gs-cAMP-protein kinase A pathway (Ji, T. H. et al. (1997) Recent Prog. Horm. Res. 52:431-453; Dufau, M. L. (1998) Annu. Rev. Physiol. 60: 461-496; Kohn, L. D. (1995) Vitam. Horm. 50: 287-384; Simoni, M. et al. (1997) Endocr. Rev. 18: 739-773).

GPCRs are of critical importance to several systems including the endocrine system, the central nervous system and peripheral physiological processes. Evolutionary analysis suggests that the ancestor of these proteins originally developed in concert with complex body plans and nervous systems. The GPCR genes and gene-products are believed to be potential causative agents of disease (Spiegel et al. (1993) J. Clin. Invest. 92:1119-1125); McKusick and Amberger (1993) J. Med Genet. 30:1-26). For example, specific defects in the rodopsin gene and the V2 vasopressin receptor gene have been shown to cause various forms of autosomal dominant and autosomal recessive retinitis pigmentosa (see Nathans et al. (1992) Annual Rev. Genet. 26:403 424), and nephrogenic diabetes insipidus (Holtzman et al. (1993) Hum. Mol. Genet. 2:1201-1204).

Given the important biological roles and properties of GPCRs, there exists a need for the identification of novel genes encoding such proteins as well as for the discovery of modulators of such molecules for use in regulating a variety of normal and/or pathological cellular processes.

### SUMMARY OF THE INVENTION

The present invention is based, at least in part, on the discovery of novel members of the G-protein coupled receptor family, referred to herein as “large G-protein coupled receptor 6” or “LGR6” nucleic acid and protein molecules. The LGR6 nucleic acid and protein molecules of the present invention are useful as targets for developing modulating agents that regulate a variety of cellular processes, e.g., neural and endocrine processes, as well as thermogenesis. Accordingly, in one aspect, this invention provides isolated nucleic acid molecules encoding LGR6 polypeptides or biologically active portions thereof, as well as nucleic acid fragments suitable as primers or hybridization probes for the detection of LGR6-encoding nucleic acids.

In one embodiment, an LGR6 nucleic acid molecule of the invention is at least 60%, 65%, 70%, 75%, 80%, 85%, 90%, 91%, 92%, 93%, 94%, 95%, 96%, 97%, 98%, 99% or more homologous to the nucleotide sequence (e.g., to the entire length of the nucleotide sequence) shown in SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, or a complement thereof.

In a preferred embodiment, the isolated nucleic acid molecule includes the nucleotide sequence shown in SEQ ID NO:1 or SEQ ID NO:3, or a complement thereof. In another embodiment, the nucleic acid molecule includes SEQ ID NO:3 and nucleotides 1-221 of SEQ ID NO:1. In yet another embodiment, the nucleic acid molecule includes SEQ ID NO:3 and nucleotides 3123-3637 of SEQ ID NO:1. In another preferred embodiment, the nucleic acid molecule has the nucleotide sequence shown in SEQ ID NO:1 or SEQ ID NO:3. In another preferred embodiment, the nucleic acid molecule includes a fragment of at least 439 nucleotides of the nucleotide sequence of SEQ ID NO:1, SEQ ID NO:3, or a complement thereof.

In another preferred embodiment, the isolated nucleic acid molecule includes the nucleotide sequence shown in SEQ ID NO:4 or SEQ ID NO:6, or a complement thereof. In another embodiment, the nucleic acid molecule includes SEQ ID NO:6 and nucleotides 1897-2486 of SEQ ID NO:4. In another preferred embodiment, the nucleic acid molecule has the nucleotide sequence shown in SEQ ID NO:4 or SEQ ID NO:6. In another preferred embodiment, the nucleic acid molecule includes a fragment of at least 481 nucleotides of the nucleotide sequence of SEQ ID NO:4, SEQ ID NO:6, or a complement thereof. In yet another preferred embodiment, the isolated nucleic acid molecule includes at least 200 consecutive nucleotides, more preferably at least 400 consecutive nucleotides, more preferably at least 600 consecutive nucleotides, more preferably at least 800 consecutive nucleotides, more preferably at least 1000 consecutive nucleotides, more preferably at least 1200 consecutive nucleotides, more preferably at least 1400 consecutive nucleotides, more preferably at least 1600 or more consecutive nucleotides of the nucleotide sequence shown SEQ ID NO:4 or 6, or a complement thereof.

In another preferred embodiment, the isolated nucleic acid molecule includes the nucleotide sequence shown in SEQ ID NO:7 or SEQ ID NO:9, or a complement thereof. In another embodiment, the nucleic acid molecule includes SEQ ID NO:9 and nucleotides 2209-2711 of SEQ ID NO:7. In another preferred embodiment, the nucleic acid molecule has the nucleotide sequence shown in SEQ ID NO:7 or SEQ ID NO:9. In another preferred embodiment, the nucleic acid molecule includes a fragment of at least 2175 nucleotides of the nucleotide sequence of SEQ ID NO:7, SEQ ID NO:9, or a complement thereof.

In another preferred embodiment, the isolated nucleic acid molecule includes the nucleotide sequence shown in SEQ ID NO:10 or SEQ ID NO:12, or a complement thereof. In another embodiment, the nucleic acid molecule includes SEQ ID NO:12 and nucleotides 1-103 of SEQ ID NO:10. In yet another embodiment, the nucleic acid molecule includes SEQ ID NO:12 and nucleotides 3005-3492 of SEQ ID NO:10. In another preferred embodiment, the nucleic acid molecule has the nucleotide sequence shown in SEQ ID NO:10 or SEQ ID NO:12. In another preferred embodiment, the nucleic acid molecule includes a fragment of at least 439 nucleotides of the nucleotide sequence of SEQ ID NO:10, SEQ ID NO:12, or a complement thereof.

In another embodiment, an LGR6 nucleic acid molecule includes a nucleotide sequence encoding a protein having an amino acid sequence sufficiently homologous to the amino acid sequence of SEQ ID NO:2, SEQ ID NO:5, SEQ ID NO:8, SEQ ID NO: 11, or an amino acid sequence encoded by the DNA insert of the plasmid deposited with ATCC as Accession Number ______, ______ or ______. In a preferred embodiment, an LGR6 nucleic acid molecule includes a nucleotide sequence encoding a protein having an amino acid sequence at least 60%, 65%, 70%, 75%, 80%, 85%, 90%, 91%, 92%, 93%, 94%, 95%, 96%, 97%, 98%, 99% or more homologous to the amino acid sequence of SEQ ID NO:2, SEQ ID NO:5, SEQ ID NO:8, SEQ ID NO:11, or the amino acid sequence encoded by the DNA insert of the plasmid deposited with ATCC as Accession Number ______, ______ or ______.

In another preferred embodiment, an isolated nucleic acid molecule encodes the amino acid sequence of a mouse or human LGR6. In yet another preferred embodiment, the nucleic acid molecule includes a nucleotide sequence encoding a protein having the amino acid sequence of SEQ ID NO: 2, SEQ ID NO:5, SEQ ID NO:8, SEQ ID NO:11, or the amino acid sequence encoded by the DNA insert of the plasmid deposited with ATCC as Accession Number ______, ______ or ______. In yet another preferred embodiment, the nucleic acid molecule is at least 1899, 2175 or 2901 nucleotides in length and encodes a protein having an LGR6 activity (as described herein).

Another embodiment of the invention features nucleic acid molecules, preferably LGR6 nucleic acid molecules, which specifically detect LGR6 nucleic acid molecules relative to nucleic acid molecules encoding non-LGR6 proteins. For example, in one embodiment, such a nucleic acid molecule is at least 439, 440, 450-500, 500-550, 550-600, 600-650, 650-700, 700-750, 750-800, 800-900, 900-1000, 1000-1500, 1500-2000, 2000-2500, 2500-3000, 3000-3500, 3500-3600 or more nucleotides in length and hybridizes under stringent conditions to a nucleic acid molecule comprising the nucleotide sequence shown in SEQ ID NO:1, or a complement thereof, or the nucleotide sequence of the DNA insert of the plasmid deposited with ATCC as Accession Number ______, or a complement thereof. In preferred embodiments, the nucleic acid molecules are at least 15 (e.g., contiguous) nucleotides in length and hybridize under stringent conditions to nucleotides 1-1381, 1427-1433, 1690-2341, 2701-2868 and 3379-3637 of SEQ ID NO:1. In other preferred embodiments, the nucleic acid molecules comprise nucleotides 1-1381, 1427-1433, 1690-2341, 2701-2868 and 3379-3637 of SEQ ID NO:1. In another preferred embodiment, the nucleic acid molecules consist of nucleotides 1-1381, 1427-1433, 1690-2341, 2701-2868 and 3379-3637 of SEQ ID NO:1.

In another particularly preferred embodiment, the nucleic acid molecule comprises a fragment of at least 481, 490-500, 500-550, 550-600, 600-650, 650-700, 700-750, 750-800, 800-900, 900-1000, 1000-1500, 1500-2000, 2000-2400 or more nucleotides in length and which hybridizes under stringent conditions to a nucleic acid molecule comprising the nucleotide sequence shown in SEQ ID NO:4, or a complement thereof, or the nucleotide sequence of the DNA insert of the plasmid deposited with ATCC as Accession Number ______, or a complement thereof. In preferred embodiments, the nucleic acid molecules are at least 15 (e.g., contiguous) nucleotides in length and hybridize under stringent conditions to nucleotides 1-1055, 1231-1290 and 1357-1722 of SEQ ID NO:4. In other preferred embodiments, the nucleic acid molecules comprise nucleotides 1-1055, 1231-1290 and 1357-1722 of SEQ ID NO:4. In another preferred embodiment, the nucleic acid molecules consist of nucleotides 1-1055, 1231-1290 and 1357-1722 of SEQ ID NO:4.

In yet another embodiment, a nucleic acid molecule of the present invention comprises a nucleotide sequence which is 167, 170-200, 200-220, 220-240, 240-260, 260-280, 280-300, 300-320, 320-340, 340-360, 360-380, 380-400, 400-420, 420-440, 440-460, 460-480, 490-500, 500-550, 537, 550-600, 600-650, 650-700, 700-750, 750-800, 800-850, 850-900, 950-1000, 1100-1200, 1200-1500, or 1500-1899 nucleotides in length and hybridizes under stringent hybridization conditions to a nucleic acid molecule comprising nucleotides 1-1899 of SEQ ID NO:4 or SEQ ID NO:6, or the nucleotide sequence of the DNA insert of the plasmid deposited with ATCC as Accession Number ______.

In another preferred embodiment, a nucleic acid molecule of the invention is at least 250-500, 500-750, 750-1000, 1000-1200, 1200-1400, 1400-1600, 1600-1800, 1800-2000, 2000-2174, 2175, 2176-2200, 2200-2400, 2400-2600, 2600 or more nucleotides in length and which hybridizes under stringent conditions to a nucleic acid molecule comprising the nucleotide sequence shown in SEQ ID NO:7 or 9, or a complement thereof, or the nucleotide sequence of the DNA insert of the plasmid deposited with ATCC as Accession Number ______, or a complement thereof.

In another preferred embodiment, a nucleic acid molecule of the invention is at least 1-50, 50-100, 100-150, 150-200, 200-250, 250-500, 500-750, 750-1000, 1000-1200, 1200-1400, 1400-1600, 1600-1800, 1800-2000, 2000-2174, 2175, 2176-2200, 2200-2400, 2400-2600, 2600 or more nucleotides in length and which hybridizes under stringent conditions to a nucleic acid molecule comprising the nucleotide sequence shown in SEQ ID NO:10 or SEQ ID NO:12, or a complement thereof, or the nucleotide sequence of the DNA insert of the plasmid deposited with ATCC as Accession Number ______, or a complement thereof. In preferred embodiments, the nucleic acid molecules are at least 15 (e.g., contiguous) nucleotides in length and hybridize under stringent conditions to SEQ ID NO:10 or SEQ ID NO:12, or a complement thereof.

In other preferred embodiments, the nucleic acid molecule encodes a naturally occurring allelic variant of a polypeptide comprising the amino acid sequence of SEQ ID NO:2, or an amino acid sequence encoded by the DNA insert of the plasmid deposited with ATCC as Accession Number ______, wherein the nucleic acid molecule hybridizes to a nucleic-acid molecule comprising SEQ ID NO:1 or SEQ ID NO:3 under stringent conditions. In yet other preferred embodiments, the nucleic acid molecule encodes a naturally occurring allelic variant of a polypeptide comprising the amino acid sequence of SEQ ID NO:5, or an amino acid sequence encoded by the DNA insert of the plasmid deposited with ATCC as Accession Number ______, wherein the nucleic acid molecule hybridizes to a nucleic acid molecule comprising SEQ ID NO:4 or SEQ ID NO:6 under stringent conditions. In another preferred embodiment, the nucleic acid molecule encodes a naturally occurring allelic variant of a polypeptide comprising the amino acid sequence of SEQ ID NO:8, or an amino acid sequence encoded by the DNA insert of the plasmid deposited with ATCC as Accession Number ______, wherein the nucleic acid molecule hybridizes to a nucleic acid molecule comprising SEQ ID NO:7 or SEQ ID NO:9 under stringent conditions. In another preferred embodiment, the nucleic acid molecule encodes a naturally occurring allelic variant of a polypeptide comprising the amino acid sequence of SEQ ID NO:11, or an amino acid sequence encoded by the DNA insert of the plasmid deposited with ATCC as Accession Number ______, wherein the nucleic acid molecule hybridizes to a nucleic acid molecule comprising SEQ ID NO:11 under stringent conditions.

Another embodiment of the invention provides an isolated nucleic acid molecule which is antisense to an LGR6 nucleic acid molecule, e.g., the coding strand of an LGR6 nucleic acid molecule.

Another aspect of the invention provides a vector comprising an LGR6 nucleic acid molecule. In certain embodiments, the vector is a recombinant expression vector. In another embodiment, the invention provides a host cell containing a vector of the invention. The invention also provides a method for producing a protein, preferably an LGR6 protein, by culturing in a suitable medium, a host cell, e.g., a mammalian host cell such as a non-human mammalian cell, of the invention containing a recombinant expression vector, such that the protein is produced.

Another aspect of this invention features isolated or recombinant LGR6 proteins and polypeptides. In one embodiment, the isolated protein, preferably an LGR6 protein, includes at least one extracellular domain. In another embodiment, the isolated protein, preferably an LGR6 protein, includes at least one leucine-rich repeat. In another embodiment, the isolated protein, preferably an LGR6 protein, includes at least one RGD cell attachment site. In another embodiment, the isolated protein, preferably an LGR6 protein, includes at least one transmembrane domain. In another embodiment, the isolated protein, preferably an LGR6 protein, includes at least one cytoplasmic domain. In another embodiment, the isolated protein, preferably an LGR6 protein, includes at least one extracellular domain, at least one leucine-rich repeat, at least one RGD cell attachment site, at least one transmembrane domain and at least one cytoplasmic domain. In another embodiment, the isolated protein, preferably an LGR6 protein, includes at least one extracellular domain; at least one leucine-rich repeat; at least one RGD cell attachment site; at least one transmembrane domain; at least one cytoplasmic domain; at least one protein phosphorylation site selected from the group consisting of a Protein Kinase C site, a Casein Kinase II site, and a tyrosine kinase phosphorylation site; at least one N-myristoylation site; and at least one glycosaminoglycan attachment site.

In a preferred embodiment, the protein, preferably an LGR6 protein, includes at least one extracellular domain, at least one leucine-rich repeat, at least one RGD cell attachment site, at least one transmembrane domain, and at least one cytoplasmic domain and has an amino acid sequence at least about 60%, 65%, 70%, 75%, 80%, 85%, 90%, 95% or more homologous to the amino acid sequence of SEQ ID NO:2, SEQ ID NO:5, SEQ ID NO:8, SEQ ID NO:11 or the amino acid sequence encoded by the DNA insert of the plasmid deposited with ATCC as Accession Number ______, ______ or ______.

In another preferred embodiment, the protein, preferably an LGR6 protein, includes at least one extracellular domain and plays a role in transducing an extracellular signal, e.g., by interacting with a ligand (e.g., a glycoprotein hormone) and/or a cell-surface receptor (e.g., an integrin receptor); by mobilizing intracellular molecules that participate in signal transduction pathways (e.g., adenylate cyclase, or phosphatidylinositol 4,5-bisphosphate (PIP₂), inositol 1,4,5-triphosphate (IP₃)); by modulating cell attachment; by maintaining energy balance and/or homeothermy, e.g., by modulating thermogenesis; by modulating endocrine function; and/or by modulating neural development and/or maintenance. In another preferred embodiment, the protein, preferably an LGR6 protein, includes at least one leucine-rich repeat and plays a role in transducing an extracellular signal, e.g., by interacting with a ligand (e.g., a glycoprotein hormone) and/or a cell surface receptor (e.g., an integrin receptor); by mobilizing intracellular molecules that participate in signal transduction pathways (e.g., adenylate cyclase, or phosphatidylinositol 4,5-bisphosphate (PIP₂), inositol 1,4,5-triphosphate (IP₃)); by modulating cell attachment; by maintaining energy balance and/or homeothermy, e.g., by modulating thermogenesis; by modulating endocrine function; and/or by modulating neural development and/or maintenance. In another preferred embodiment, the protein, preferably an LGR6 protein, includes at east one RGD cell attachment site and plays a role in transducing an extracellular signal, e.g., by interacting with a ligand (e.g., a glycoprotein hormone) and/or a cell surface receptor (e.g., an integrin receptor); by mobilizing intracellular molecules that participate in signal transduction pathways (e.g., adenylate cyclase, or phosphatidylinositol 4,5-bisphosphate (PIP₂), inositol 1,4,5-triphosphate (IP₃)); by modulating cell attachment; by maintaining energy balance and/or homeothermy, e.g., by modulating thermogenesis; by modulating endocrine function; and/or by modulating neural development and/or maintenance.

In another preferred embodiment, the protein, preferably an LGR6 protein, includes at least one transmembrane domain and plays a role in transducing an extracellular signal, e.g., by interacting with a ligand (e.g., a glycoprotein hormone) and/or a cell surface receptor (e.g., an integrin receptor); by mobilizing intracellular molecules that participate in signal transduction pathways (e.g., adenylate cyclase, or phosphatidylinositol 4,5-bisphosphate (PIP₂), inositol 1,4,5-triphosphate (IP₃)); by modulating cell attachment; by maintaining energy balance and/or homeothermy, e.g., by modulating thermogenesis; by modulating endocrine function; and/or by modulating neural development and/or maintenance. In another preferred embodiment, the protein, preferably an LGR6 protein, includes at least one cytoplasmic domain and plays a role in transducing an extracellular signal, e.g., by interacting with a ligand (e.g., a glycoprotein hormone) and/or a cell surface receptor (e.g., an integrin receptor); by mobilizing intracellular molecules that participate in signal transduction pathways (e.g., adenylate cyclase, or phosphatidylinositol 4,5-bisphosphate (PIP₂), inositol 1,4,5-triphosphate (IP₃)); by modulating cell attachment; by maintaining energy balance and/or homeothermy, e.g., by modulating thermogenesis; by modulating endocrine function; and/or by modulating neural development and/or maintenance.

In another preferred embodiment, the protein, preferably an LGR6 protein, includes at least one extracellular domain, at least one leucine-rich repeat, at least one RGD-cell attachment site, at least one transmembrane domain and at least one cytoplasmic domain, and plays a role in in transducing an extracellular signal, e.g., by interacting with a ligand (e.g., a glycoprotein hormone) and/or a cell surface receptor (e.g., an integrin receptor); by mobilizing intracellular molecules that participate in signal transduction pathways (e.g., adenylate cyclase, or phosphatidylinositol 4,5-bisphosphate (PIP₂), inositol 1,4,5-triphosphate (IP₃)); by modulating cell attachment; by maintaining energy balance and/or homeothermy, e.g., by modulating thermogenesis; by modulating endocrine function; and/or by modulating neural development and/or maintenance.

In one preferred embodiment, the isolated protein includes at least 50 consecutive amino acids, more preferably at least 100 consecutive amino acids, more preferably at least 150 consecutive amino acids, more preferably at least 200 consecutive amino acids, more preferably at least 250 consecutive amino acids, more preferably at least 350 consecutive amino acids, more preferably at least 450 consecutive amino acids, more preferably at least 500 consecutive amino acids of the amino acid sequence shown SEQ ID NO:5, 8 or 11.

In yet another preferred embodiment, the protein, preferably an LGR6 protein, includes at least one leucine-rich repeat, at least one RGD-cell attachment site, at least one transmembrane domain and at least one cytoplasmic domain, and is encoded by a nucleic acid molecule having a nucleotide sequence which hybridizes under stringent hybridization conditions to a nucleic acid molecule comprising the nucleotide sequence of SEQ ID NO:1, SEQ ID NO:3, SEQ ID NO:4, SEQ ID NO:6, SEQ ID NO:7, SEQ ID NO:9, SEQ ID NO:10 or SEQ ID NO:12.

In another embodiment, the invention features fragments of the proteins having the amino acid sequence of SEQ ID NO:2, SEQ ID NO:5, SEQ ID NO:8, SEQ ID NO:11 wherein the fragment comprises at least 15 amino acids (e.g., contiguous amino acids) of the amino acid sequence of SEQ ID NO:2, SEQ ID NO:5, SEQ ID NO:8, SEQ ID NO:11 or an amino acid sequence encoded by the DNA insert of the plasmid deposited with the ATCC as Accession Number ______, ______ or ______. In another embodiment, the protein, preferably an LGR6 protein, has the amino acid sequence of SEQ ID NO:2, SEQ ID NO:5, SEQ ID NO:8 or SEQ ID NO:11.

In another embodiment, the invention features an isolated protein, preferably an LGR6 protein, which is encoded by a nucleic acid molecule having a nucleotide sequence at least about 60%, 65%, 70%, 75%, 80%, 85%, 90%, 95% or more homologous to a nucleotide sequence of SEQ ID NO:1, SEQ ID NO:3, or a complement thereof. In yet another embodiment, the invention features an isolated protein, preferably an LGR6 protein, which is encoded by a nucleic acid molecule having a nucleotide sequence at least about 60%, 65%, 70%, 75%, 80%, 85%, 90%, 95% or more homologous to a nucleotide sequence of SEQ ID NO:4, SEQ ID NO:6, or a complement thereof. In yet another embodiment, the invention features an isolated protein, preferably an LGR6 protein, which is encoded by a nucleic acid molecule having a nucleotide sequence at least about 60%, 65%, 70%, 75%, 80%, 85%, 90%, 95% or more homologous to a nucleotide sequence of SEQ ID NO:7, SEQ ID NO:9, or a complement thereof. In yet another embodiment, the invention features an isolated protein, preferably an LGR6 protein, which is encoded by a nucleic acid molecule having a nucleotide sequence at least about 60%, 65%, 70%, 75%, 80%, 85%, 90%, 95% or more homologous to a nucleotide sequence of SEQ ID NO:10, SEQ ID NO:12, or a complement thereof. This invention further features an isolated protein, preferably an LGR6 protein, which is encoded by a nucleic acid molecule having a nucleotide sequence which hybridizes under stringent hybridization conditions to a nucleic acid molecule comprising the nucleotide sequence of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or a complement thereof.

The proteins of the present invention or biologically active portions thereof, can be operatively linked to a non-LGR6 polypeptide (e.g., heterologous amino acid sequences) to form fusion proteins. The invention further features antibodies, such as monoclonal or polyclonal antibodies, that specifically bind proteins of the invention, preferably LGR6 proteins. In addition, the LGR6 proteins or biologically active portions thereof can be incorporated into pharmaceutical compositions, which optionally include pharmaceutically acceptable carriers.

In another aspect, the present invention provides a method for detecting the presence of an LGR6 nucleic acid molecule, protein or polypeptide in a biological sample by contacting the biological sample with an agent capable of detecting an LGR6 nucleic acid molecule, protein or polypeptide such that the presence of an LGR6 nucleic acid molecule, protein or polypeptide is detected in the biological sample.

In another aspect, the present invention provides a method for detecting the presence of LGR6 activity in a biological sample by contacting the biological sample with an agent capable of detecting an indicator of LGR6-activity such that the presence of LGR6 activity is detected in the biological sample.

In another aspect, the invention provides a method for modulating LGR6 activity comprising contacting a cell capable of expressing LGR6 with an agent that modulates LGR6 activity such that LGR6 activity in the cell is modulated. In one embodiment, the agent inhibits LGR6 activity. In another embodiment, the agent stimulates LGR6 activity. In one embodiment, the agent is an antibody that specifically binds to an LGR6 protein. In another embodiment, the agent modulates expression of LGR6 by modulating transcription of an LGR6 gene or translation of an LGR6 mRNA. In yet another embodiment, the agent is a nucleic acid molecule having a nucleotide sequence that is antisense to the coding strand of an LGR6 mRNA or an LGR6 gene.

In one embodiment, the methods of the present invention are used to treat a subject having a disorder characterized by aberrant LGR6 protein or nucleic acid expression or activity by administering an agent which is an LGR6 modulator to the subject. In one embodiment, the LGR6 modulator is an LGR6 protein. In another embodiment the LGR6 modulator is an LGR6 nucleic acid molecule. In yet another embodiment, the LGR6 modulator is a peptide, peptidomimetic, or other small molecule. In a preferred embodiment, the disorder characterized by aberrant LGR6 protein or nucleic acid expression is a weight disorder, e.g., obesity, anorexia, cachexia; a neural disorder, e.g., a CNS disorder, including Alzheimer's disease; an endocrine disorder; or a cardiovascular disorder, e.g., atherosclerosis, ischaemia reperfusion injury, cardiac hypertrophy, hypertension, coronary artery disease, myocardial infarction, arrythmia, cardiomyopathies, and congestive heart failure.

The present invention also provides a diagnostic assay for identifying the presence or absence of a genetic alteration characterized by at least one of (i) aberrant modification or mutation of a gene encoding an LGR6 protein; (ii) mis-regulation of the gene; and (iii) aberrant post-translational modification of an LGR6 protein, wherein a wild-type form of the gene encodes a protein with an LGR6 activity.

In another aspect the invention provides a method for identifying a compound that binds to or modulates the activity of an LGR6 protein, by providing an indicator composition comprising an LGR6 protein having LGR6 activity, contacting the indicator composition with a test compound, and determining the effect of the test compound on LGR6 activity in the indicator composition to identify a compound that modulates the activity of an LGR6 protein.

Other features and advantages of the invention will be apparent from the following detailed description and claims.

### BRIEF DESCRIPTION OF THE DRAWINGS

FIG. 1 depicts a mouse cDNA sequence (SEQ ID NO:1) and predicted amino acid sequence (SEQ ID NO:2) of mouse LGR6 (also referred to herein by clone designation “ftmzb048h10”). The methionine-initiated open reading frame of mouse ftmzb048h10 (without the 5′ and 3′ untranslated regions) extends from nucleotide 222 to nucleotide 3122 of SEQ ID NO: 1 (shown herein as SEQ ID NO:3).

FIG. 2 depicts an alignment of portions of the amino acid sequence of the mouse LGR6 (clone ftmzb048h10) and a leucine-rich repeat consensus sequence derived from a hidden Markov model (PF00560). Alignments of eight leucine-rich regions of mouse LGR6 are indicated. For each alignment, the upper sequence is the PF00560 sequence while the lower sequence corresponds to amino acids 67 to 114, 115 to 162, 163 to 210, 211 to 257, 258 to 305, 306 to 352, 353 to 398 and 399 to 446 of SEQ ID NO:2.). The leucine-rich consensus sequence contains two leucine-rich repeats. Thus, the total number of leucine-rich repeats is sixteen, instead of eight.

FIG. 3 is a table summarizing proteins with leucine-rich repeats based on function, cellular location, length, leucine-rich consensus sequence and accession number. This table was obtained from Kobe, B. and Deisenhofer, J. (1994) Trends in Biochem Sci. at page 416. The numbers above the sequences indicate the position in the repeat in reference to the consensus of porcine RNase inhibitor. One-letter code is used for amino acids. An amino acid is included in the consensus if present at that position in more than half of the repeats; ‘a’ represents A, V, L, F, Y or M, and is included in the consensus if these amino acids are present at that position in more than 80% of the repeats. Symbols used ‘,’, any amino acid; ‘−’, gap; ‘+’, amino acid may or may not be present at this position.

The following abbreviations are used: RNase, ribonuclease; GP, glycoprotein; snRNP, small nuclear ribonucleoprotein particle; ECM, extracellular matrix; PM plasma membrane; EC, extracellular; TGF, transforming growth factor; IC, intracellular, BMP, bone-morpfogenic protein; WF, von Willebrand factor; LPS-LPB, complex of lipopolysaccharide and lipopolysaccharide-binding protein; NGF, nerve growth factor; BDNF, brain-derived neurotrophic factor; NT-3, neurotrophin-3; LH, lutrophin; CG, choriogonadotrophin; FSH, follitrophin; TSH, thyrotrophin; T-LR trypsnosomal leucine-rich protein; RM membrane, rough microsoal membrane. Total number of repeats is the number of occurrences of the a.a.a.N/C/T sequence, where ‘a’ represents A, V, L, F, Y or M; repeats shorter than 18 residues and isolated single repeats were not counted. Only the counted repeats were used to determine the consensus sequence.

FIG. 4 depicts a human cDNA sequence (SEQ ID NO:4) of human LGR6 (also referred to herein by clone designation “fahr”). The methionine-initiated open reading frame of human fahr (without the 5′ and 3′ untranslated regions) extends from nucleotide 1 to nucleotide 1899 of SEQ ID NO:4 (shown herein as SEQ ID NO:6).

FIG. 5 depicts the predicted amino acid sequence (SEQ ID NO:5) of human LGR6 (clone fahr).

FIG. 6 depicts an alignment of a portion of the amino acid sequence of the human LGR6 (clone fahr) and a leucine-rich repeat consensus sequence derived from a hidden Markov model (PF00560). The upper sequence in the alignment is the PF00560 sequence while the lower sequence corresponds to amino acids 64 to 111 of SEQ ID NO:5. The leucine-rich consensus sequence contains two leucine-rich repeats. Thus, the total number of leucine-rich repeats is two, instead of one.

FIG. 7 depicts a multiple sequence alignment of the amino acid sequence of mouse LGR6 (clone ftmzb048h10), clone aambb001d112 and human LGR6 (clone fahr). The approximate location of the seven transmembrane domains (1-VII) is indicated.

FIG. 8 depicts a partial cDNA sequence and predicted amino acid sequence of human LGR6. The nucleotide sequence corresponds to nucleic acids 1 to 2711 of SEQ ID NO:7. The amino acid sequence corresponds to amino acids 1 to 736 of SEQ ID NO: 8. The coding region without the and 3′ untranslated region of the human LGR6 gene is shown in SEQ ID NO:9.

FIG. 9 depicts a structural, hydrophobicity, and antigenicity analysis of the human LGR6 protein (SEQ ID NO:11).

FIG. 10 depicts the results of a search which was performed against the HMM database (PFAM) using the amino acid sequence human LGR6 (SEQ ID NO:11) which resulted in the identification of “Leucine rich repeat (LRR) domains” and “7 transmembrane receptor (rhodopsin family) domains” in the human LGR6 protein.

FIG. 11 depicts the results of a search which was performed against the HMM database (SMART) using the amino acid sequence human LGR6 (SEQ ID NO:11) which resulted in the identification of a “Leucine rich repeat (LRR) domains”, for example, typical LRR (LRR_typ_{—}2), bacterial type LRR (LRR_bac_{—}2), SDS22-like LRR (LRR_sd22_{—}2), and plant specific LRR (LRR_PS_{—}2) in the human LGR6 protein.

FIG. 12 depicts a local alignment of the mouse LGR6 nucleic acid sequence with the human LGR6 nucleic acid sequence using the the GAP program in the GCG software package, using a nwsgapdna matrix, a gap weight of 12 and a length weight of 4. The results showed a 84.211% identity between the two sequences.

FIG. 13 depicts a local alignment of the mouse LGR6 protein with the human LGR6 protein using the the GAP program in the GCG software package, using a Blossum 62 matrix and a gap weight of 12 and a length weight of 4. The results showed a 89.281% identity between the two sequences.

FIG. 14 depicts the nucleotide sequence of the full length human LGR6 (SEQ ID NO:10) (also referred to herein by clone designation “Fbh150881”).

FIG. 15 depicts the predicted amino acid sequence of human LGR6 (SEQ ID NO:11) (also referred to herein by clone designation “Fbh150881”).

FIG. 16 depicts depicts a local alignment of the mouse LGR6 protein with the full length human LGR6 protein using the the GAP program in the GCG software package, using a Blossum 62 matrix and a gap weight of 12 and a length weight of 4. The results showed a 89.855% identity between the two sequences.

### DETAILED DESCRIPTION OF THE INVENTION

The present invention is based, at least in part, on the discovery of novel molecules, referred to herein as LGR6 nucleic acid and protein molecules, which are members of G-protein coupled receptor family (GPCR). These novel molecules are capable of, for example, interacting with an extracellular signal ligand (e.g., a glycoprotein hormone) and/or a cell surface receptor (e.g., an integrin receptor), and thereby modulating cellular processes including cell attachment, mobilization of signal transduction pathways, regulation of energy balance and/or homeothermy, as well as modulation of endocrine function, and/or neural development and maintenance.

The LGR6 molecules of the present invention comprise a family of molecules having certain conserved structural and functional features. The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics.

As used herein, the term “G protein-coupled receptor” or “GPCR” refers to a family of proteins that preferably comprise an N-terminal extracellular domain, seven transmembrane domains (also referred to as membrane-spanning domains), three extracellular domains (also referred to as extracellular loops), three cytoplasmic domains (also referred to as cytoplasmic loops), and a C-terminal cytoplasmic domain (also referred to as a cytoplasmic tail). Members of the GPCR family also share certain conserved amino acid residues, some of which have been determined to be critical to receptor function and/or G protein signaling.

For example, GPCRs usually contain the following features including a conserved asparagine residue in the first transmembrane domain; a cysteine residue in the first extracellular loop which is believed to form a disulfide bond with a conserved cysteine residue in the second extracellular loop; a conserved phenylalanine residue which is commonly found as part of the motif FXXCXXP; and a conserved leucine residue in the seventh transmembrane domain which is commonly found as part of the motif DPXXY or NPXXY. An alignment of the transmembrane domains of 44 representative GPCRs can be found at http://mgdkk1.nidll.nih.gov:8000/extended.html.

The LGR6 proteins of the present invention contain a significant number of structural characteristics in common with members of the GPCR family. For example, the mouse LGR6 protein (clone ftmzb048h10) contains conserved cysteines found in the first two extracellular loops (prior to the third and fifth transmembrane domains, respectively) of most GPCR (e.g., cys 642 and cys 717 of SEQ ID NO:2). Similarly, the human LGR6 protein (clone fahr) contains conserved cysteine residues at positions 308 and 383 of SEQ ID NO: 5. The human LGR6 protein (clone fahr) contains conserved cysteine residues at positions 411 and 486 of SEQ ID NO: 8. The human LGR6 protein (clone Fbh150881) contains conserved systeine residues at positions 642 and 717 of SEQ ID NO:11. The two cysteine residues are believed to form a disulfide bond that stabilizes the functional protein structure. In addition, both mouse and human LGR6 proteins contain an NPXXY in the seventh transmembrane domain (e.g., residues 823-827 of SEQ ID NO:2, residues 489-493 of SEQ ID NO:5, residues 592-596 of SEQ ID NO:8, and residues 823-827 of SEQ ID NO:11, respectively).

Based on structural similarities, members of the GPCR family have been classified into various subfamilies, including: Subfamily I which comprises receptors typified by rhodopsin and the beta2-adrenergic receptor and currently contains over 200 unique members (reviewed by Dohlman et al. (1991) Annu. Rev. Biochem. 60:653-688); Subfamily II, which includes the parathyroid hormone/calcitonin/secretin receptor family (Juppner et al (1991) Science 254:1024-1026; Lin et al (1991) Science 254:1022-1024); Subfamily III, which includes the metabotropic glutamate receptor family in mammals, such as the GABA receptors (Nakanishi et al. (1992) Science 258: 597-603); Subfamily IV, which includes the cAMP receptor family that is known to mediate the chemotaxis and development of D. discoideum (Klein et al. (1988) Science 241:1467-1472); and Subfamily V, which includes the fungal mating pheromone receptors such as STE2 (reviewed by Kurjan I et al. (1992) Annu. Rev. Biochem. 61:1097-1129). Within each family, distinct, highly conserved motifs have been identified. These motifs have been suggested to be critical for the structural integrity of the receptor, as well as for coupling to G proteins.

The LGR6 proteins of the present invention show significant homology to a subgroup of the Subfamily I of GPCRs represented by the glycoprotein hormone receptors. As used herein, the term “glycoprotein hormone receptors” refers to a subgroup of GPCRs which share certain structural and functional characteristics. For example, glycoprotein hormone receptors have an extended N-terminal extracellular (ecto-) domain which contains several leucine-rich repeats. The ligands for these receptors are glycoprotein hormones such as gonadotropins (e.g., luteinizing hormone (LH), follicle-stimulating hormone (FSH), choriogonadotropin (CG) and thyroid-stimulating hormone (TSH)). Binding of a glycoprotein hormone to these receptors leads to activation of the Gs-cAMP-protein kinase A pathway (Ji, T. H. et al. (1997) Recent Prog. Horm. Res. 52:431-453; Dufau, M. L. (1998) Annu. Rev. Physiol. 60: 461-496; Kohn, L. D. (1995) Vitam. Horm. 50: 287-384; Simoni, M. et al. (1997) Endocr. Rev. 18: 739-773). In particular, the LGR6 proteins of the invention show significant homology to two orphan receptors termed LGR4 and LGR5 (Hsu, J. W. et al. (1988) Mol. Endocrinol. 12 (12): 1830-1845; Accession Nos. AF0661443 and AF061444, respectively).

In one embodiment, the LGR6 proteins of the present invention have an amino acid sequence of about 400-1100, preferably about 500-1000, and more preferably about 600-970 amino acids in length. For example, the LGR6 proteins preferably include an N-terminal extracellular domain which contains at least one, two, three, four, five, six, seven, eight, nine, ten, eleven, twelve, thirteen, fourteen, fifteen, and preferably sixteen leucine-rich repeats; and at least one RGD attachment site. Preferably, the LGR6 protein further includes at least one, two, three, four, five, six or seven transmembrane domains (also referred to as membrane-spanning domains), at least one, two, and preferably, three extracellular domains (also referred to as extracellular loops), at least one, two and preferably, three cytoplasmic domains (also referred to as cytoplasmic loops), and at least one C-terminal cytoplasmic domain (also referred to as a cytoplasmic tail).

In one embodiment, an LGR6 protein includes at least one extracellular domain. When located at the N-terminal domain the extracellular domain is referred to herein as an “N-terminal extracellular domain”, or as an N-terminal extracellular loop in the amino acid sequence of the protein. As used herein, an “N-terminal extracellular domain” includes an amino acid sequence having about 1-700, preferably about 1-650, more preferably about 1-600, more preferably about 1-560, even more preferably about 1-563 amino acid residues in length and is located outside of a cell or extracellularly. The C-terminal amino acid residue of a “N-terminal extracellular domain” is adjacent to an N-terminal amino acid residue of a transmembrane domain in a naturally-occurring LGR6 or LGR6-like protein. For example, an N-terminal cytoplasmic domain is located at about amino acid residues 1-563 of SEQ ID NO:2. Preferably, the N-terminal extracellular domain is capable of interacting (e.g., binding to) with an extracellular signal, for example, a ligand (e.g., a glycoprotein hormone) or a cell surface receptor (e.g., an integrin-receptor). Most preferably, the N-terminal extracellular domain mediates protein-protein interactions, signal transduction and/or cell adhesion.

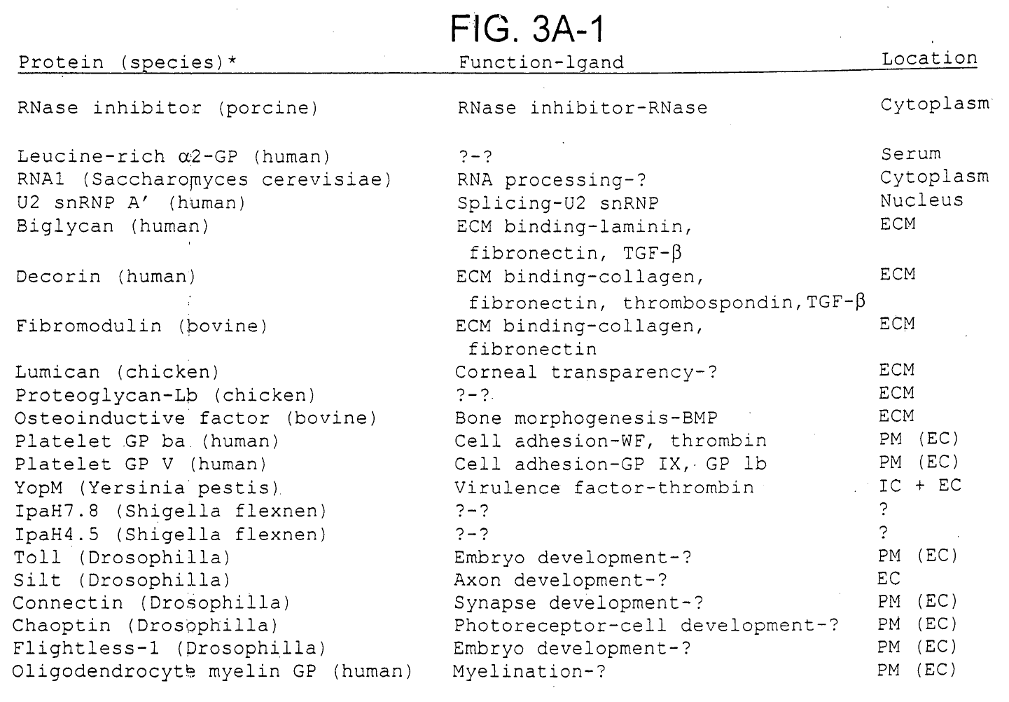

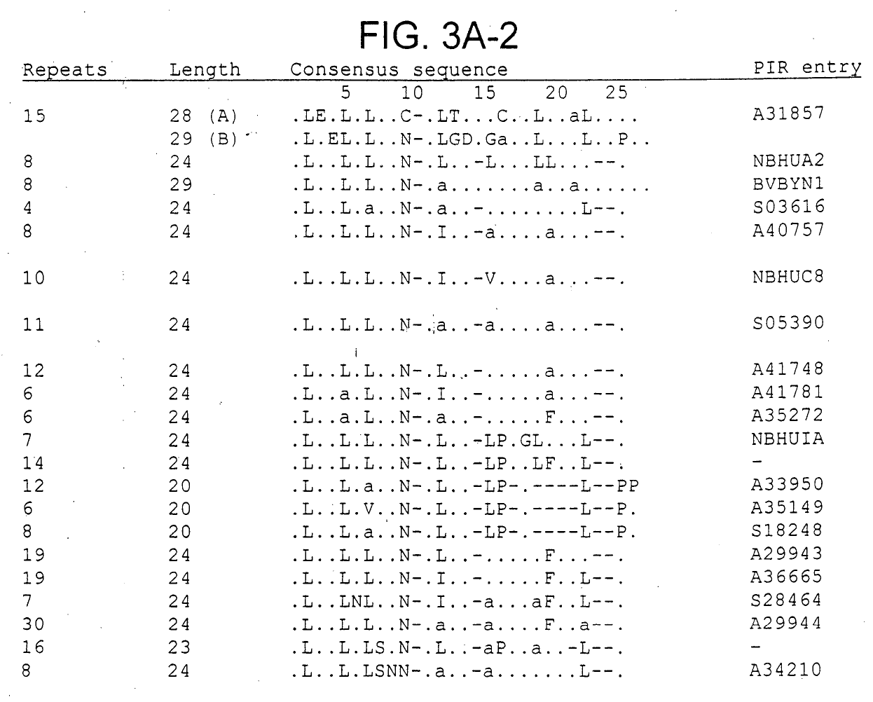

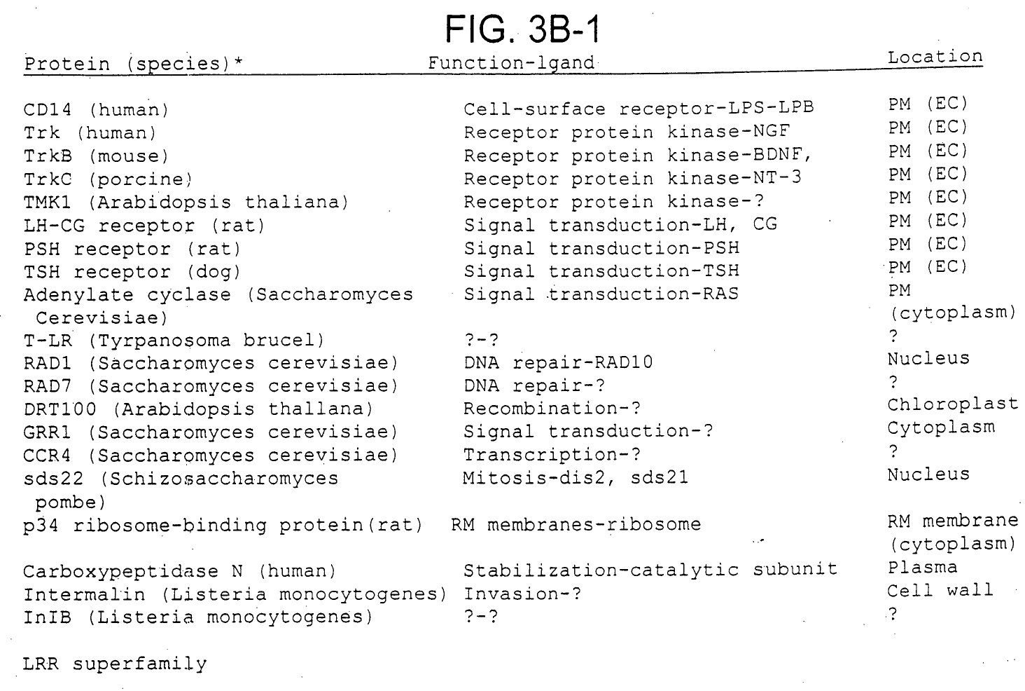

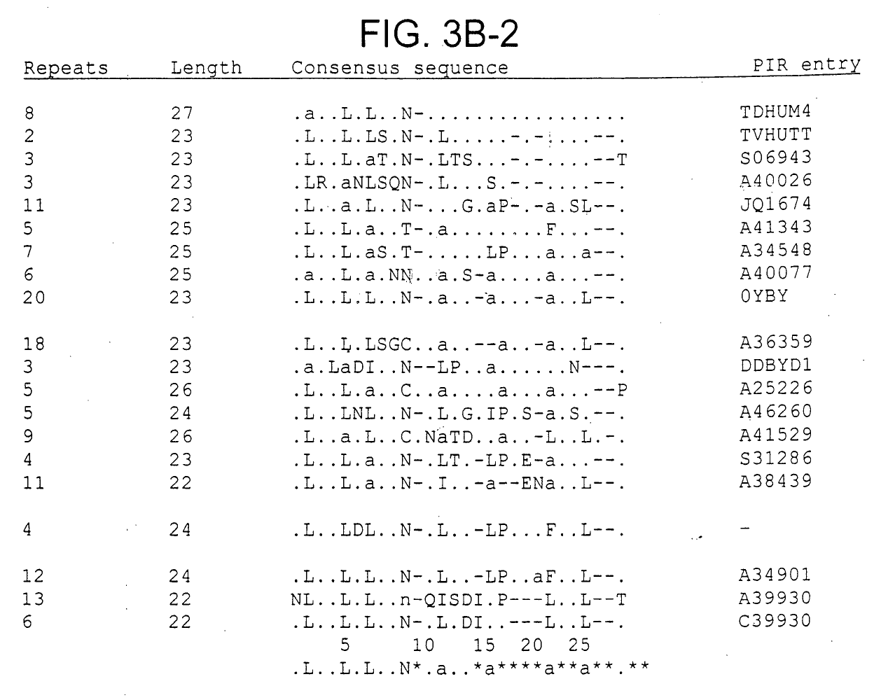

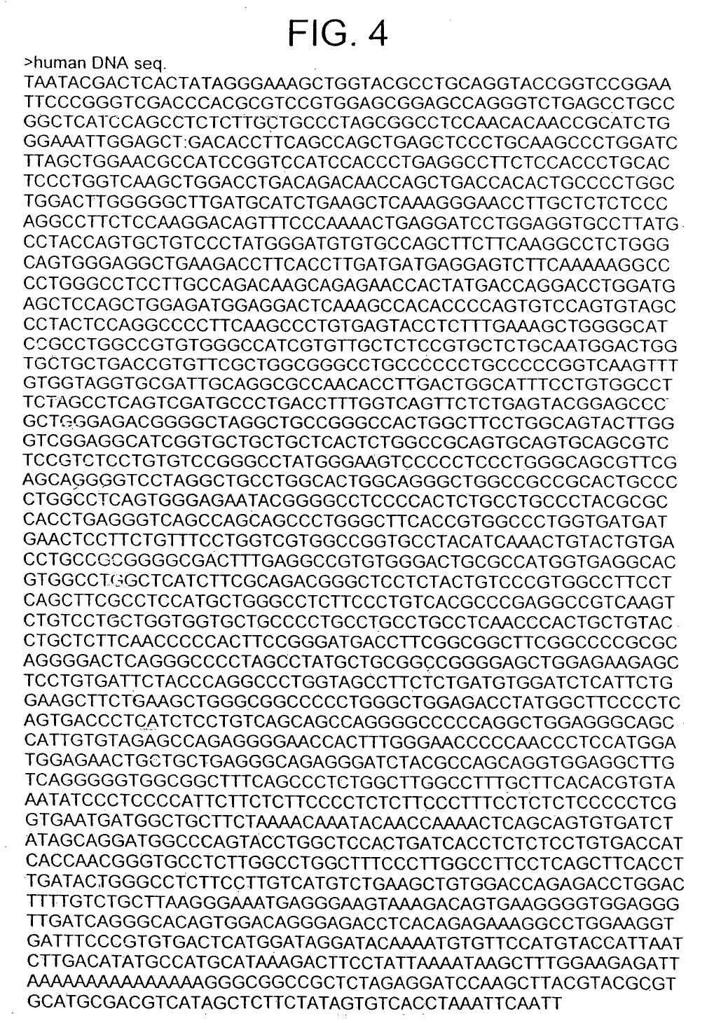

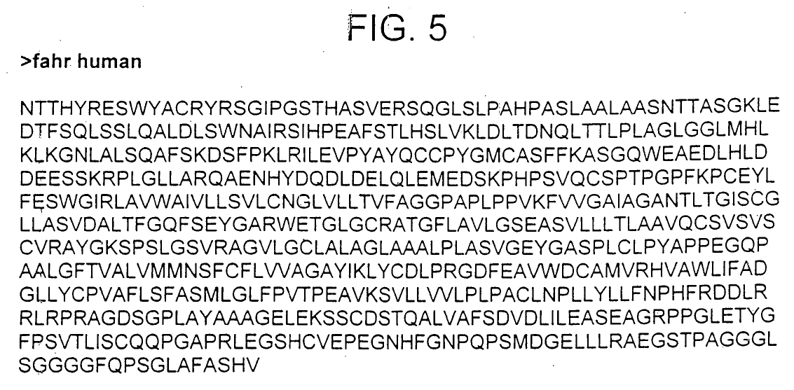

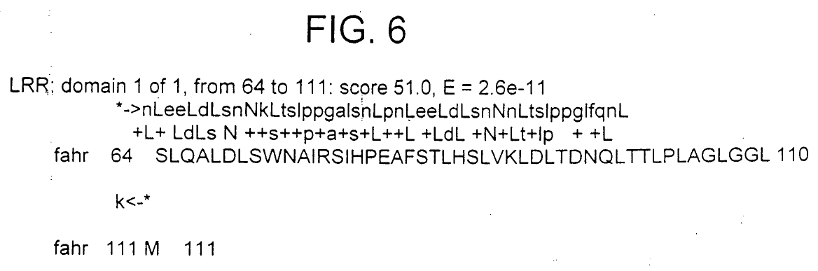

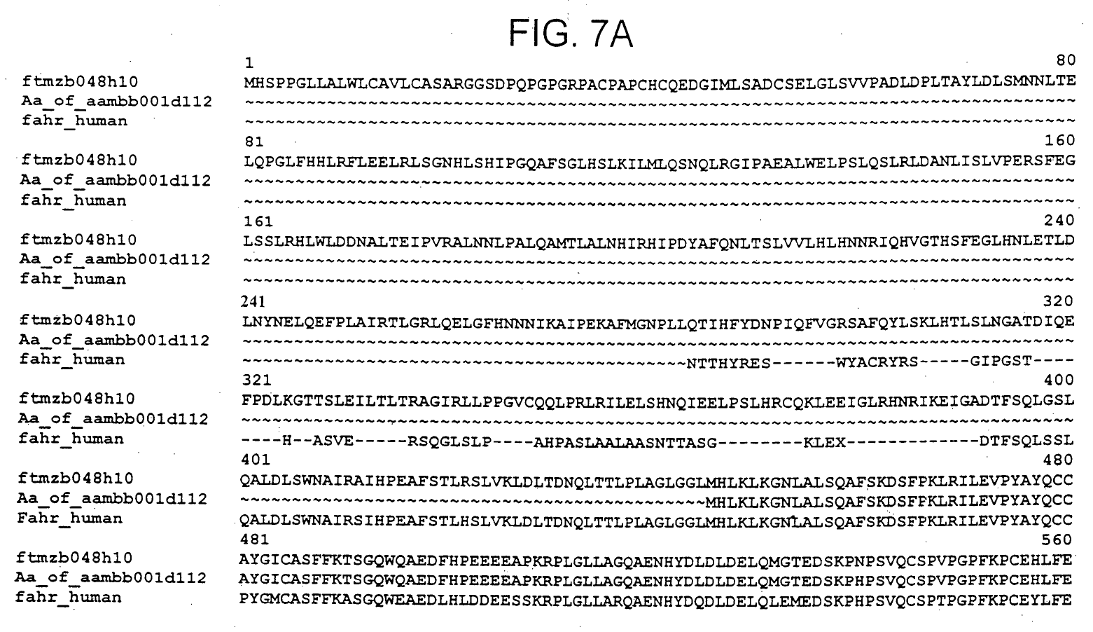

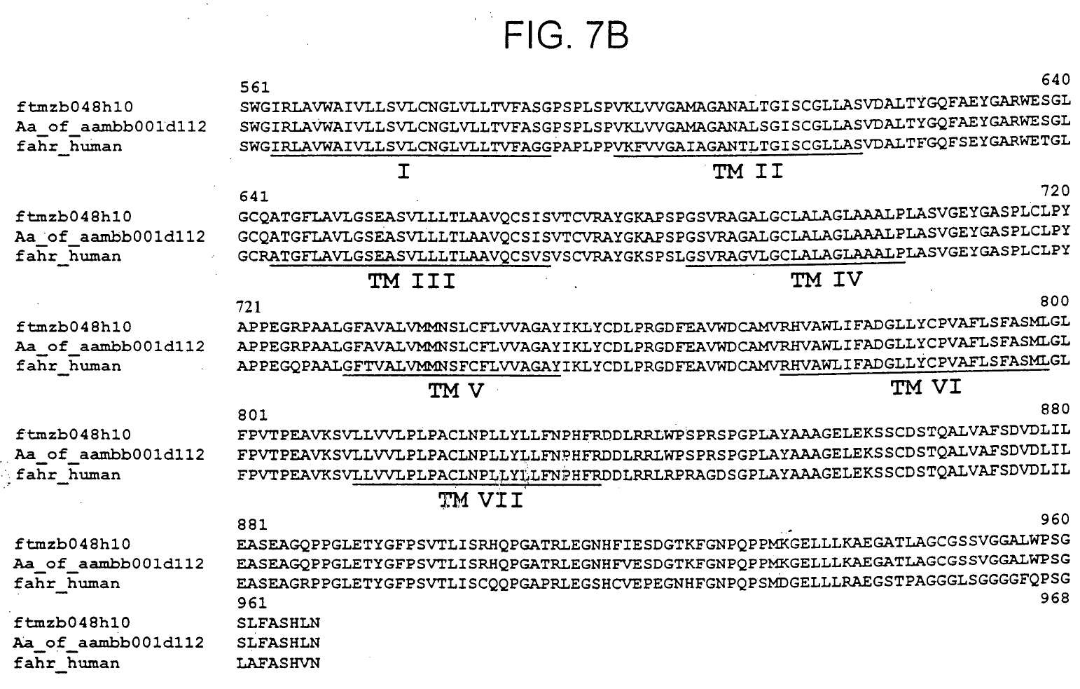

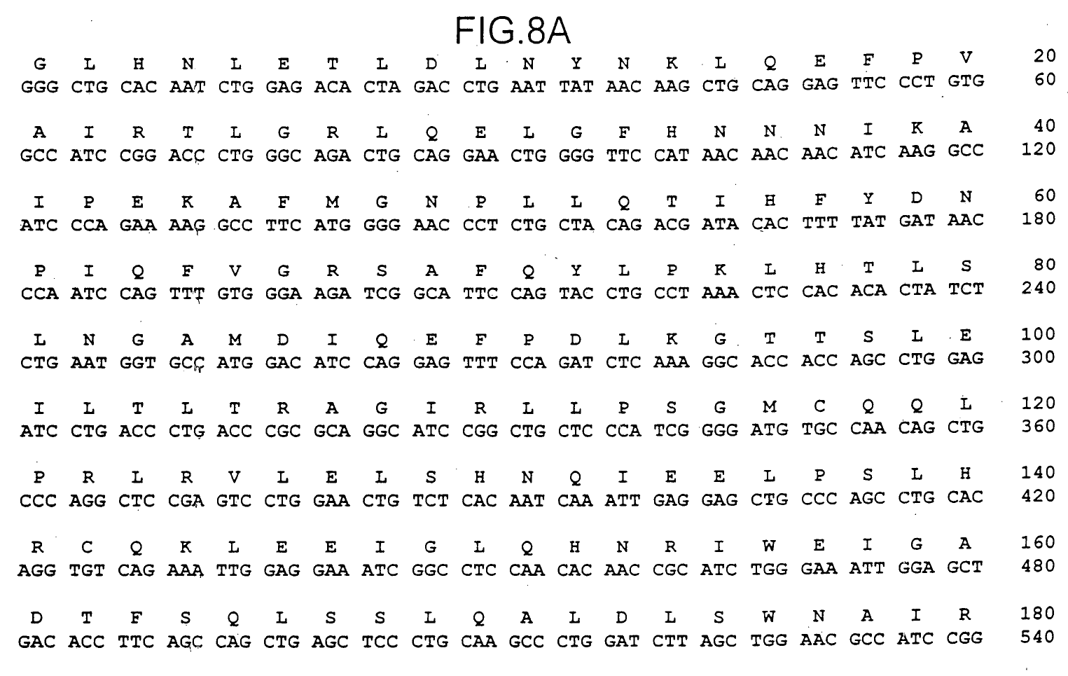

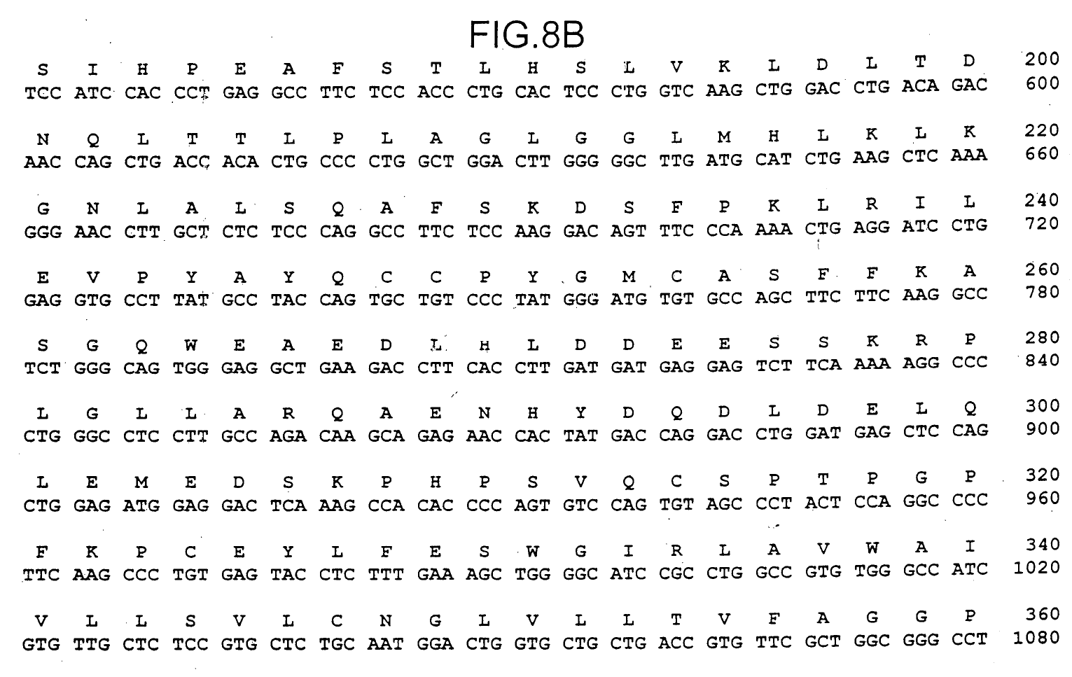

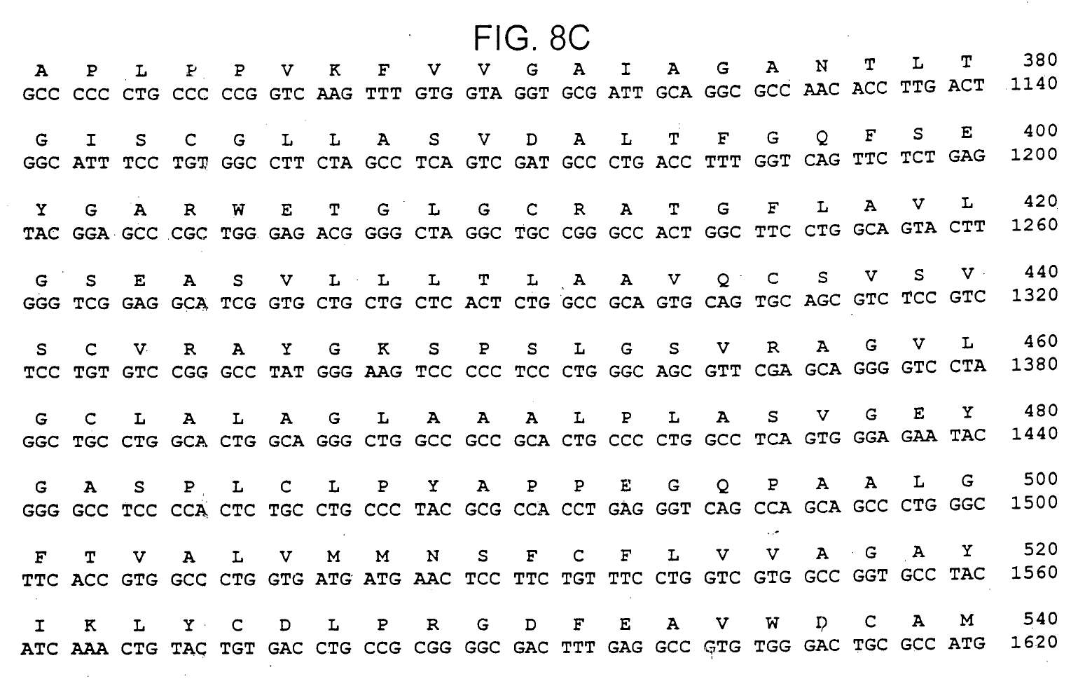

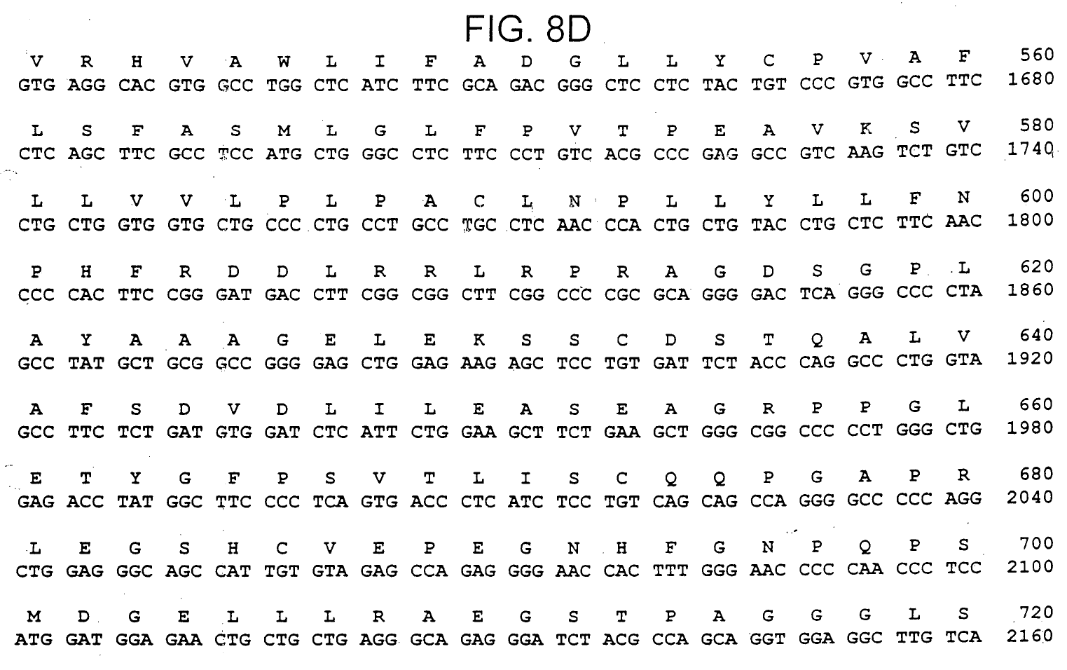

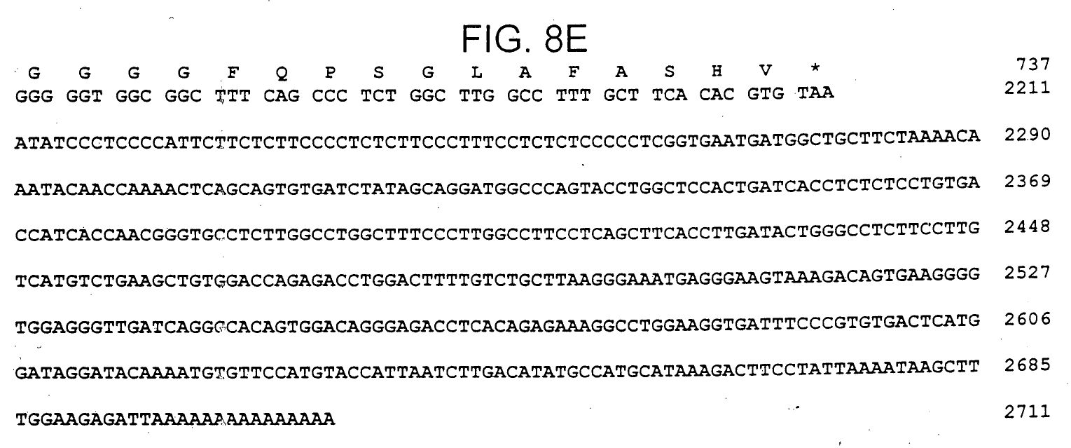

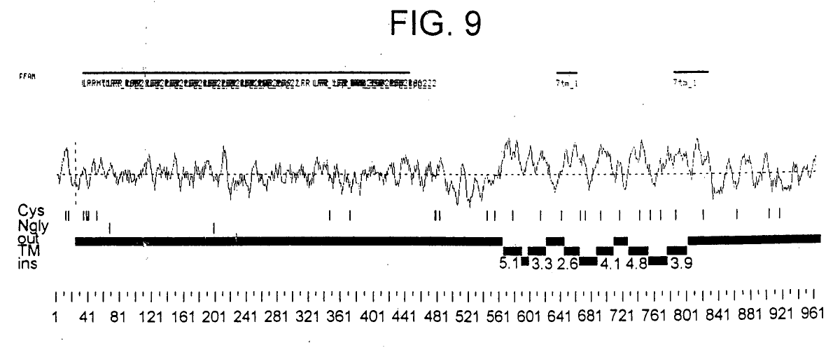

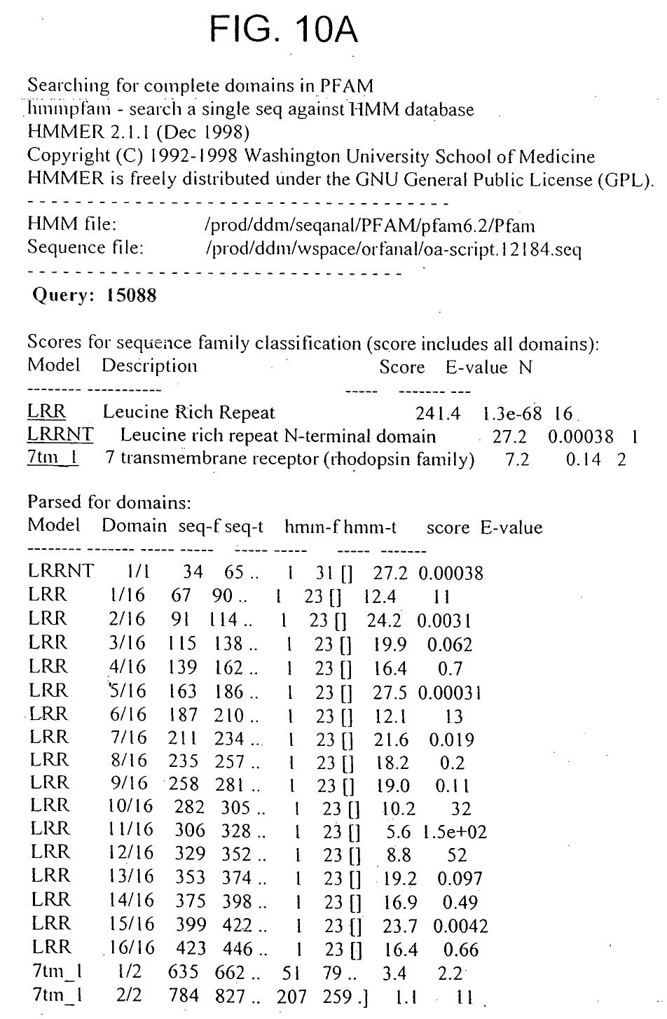

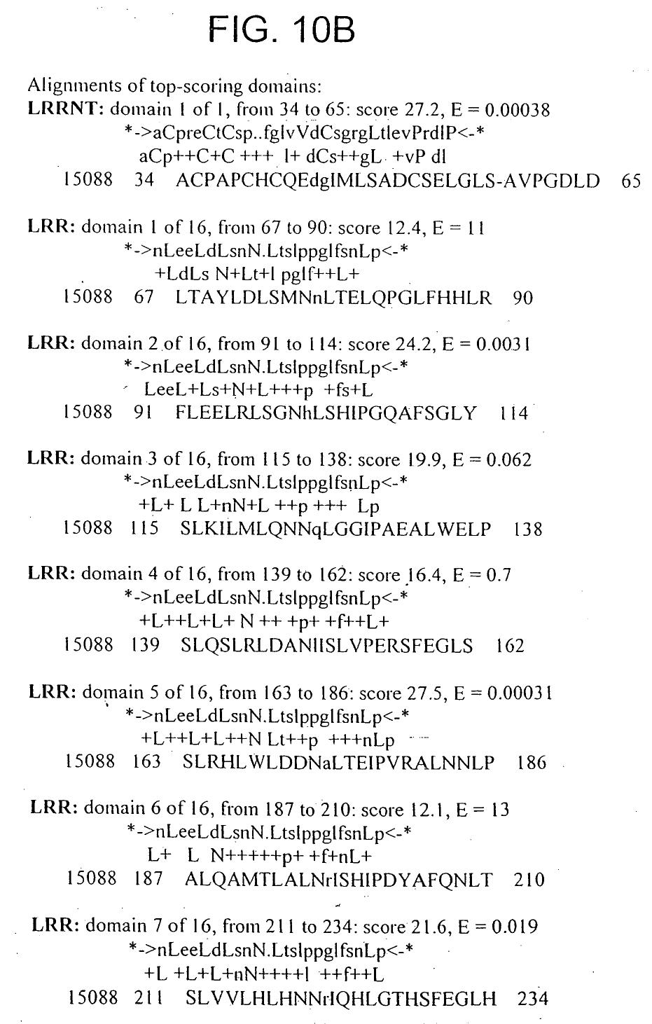

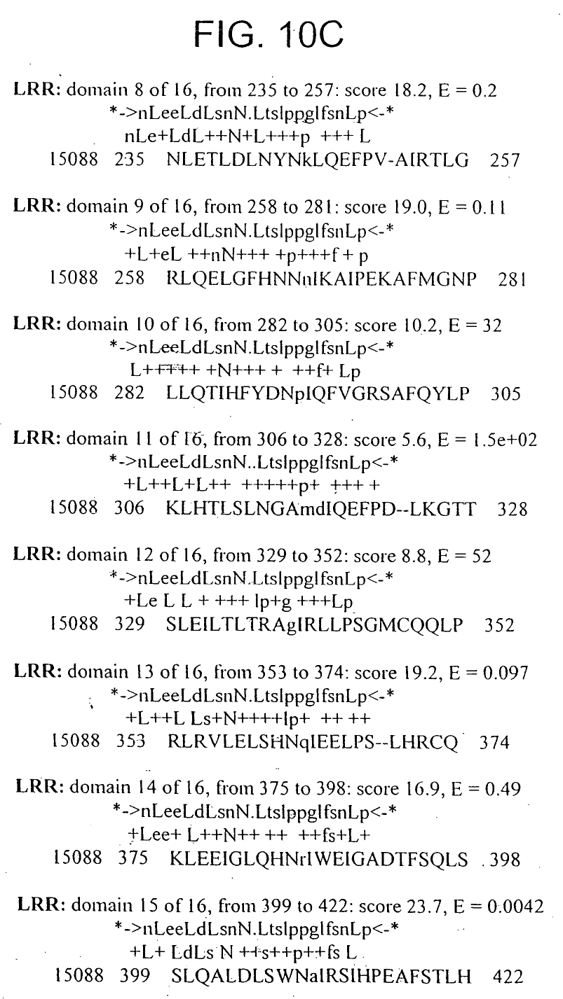

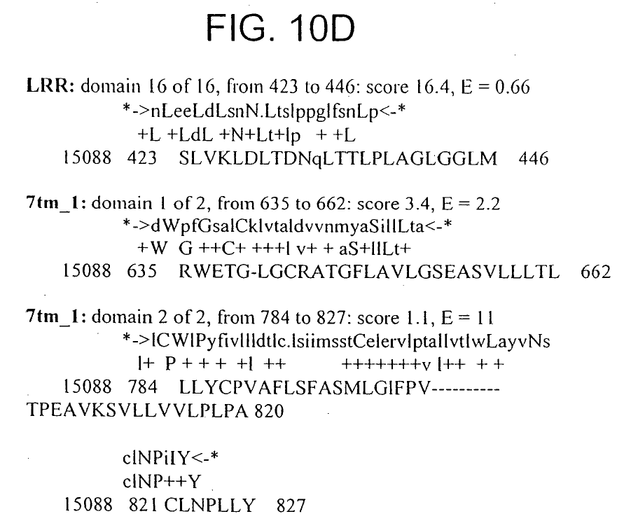

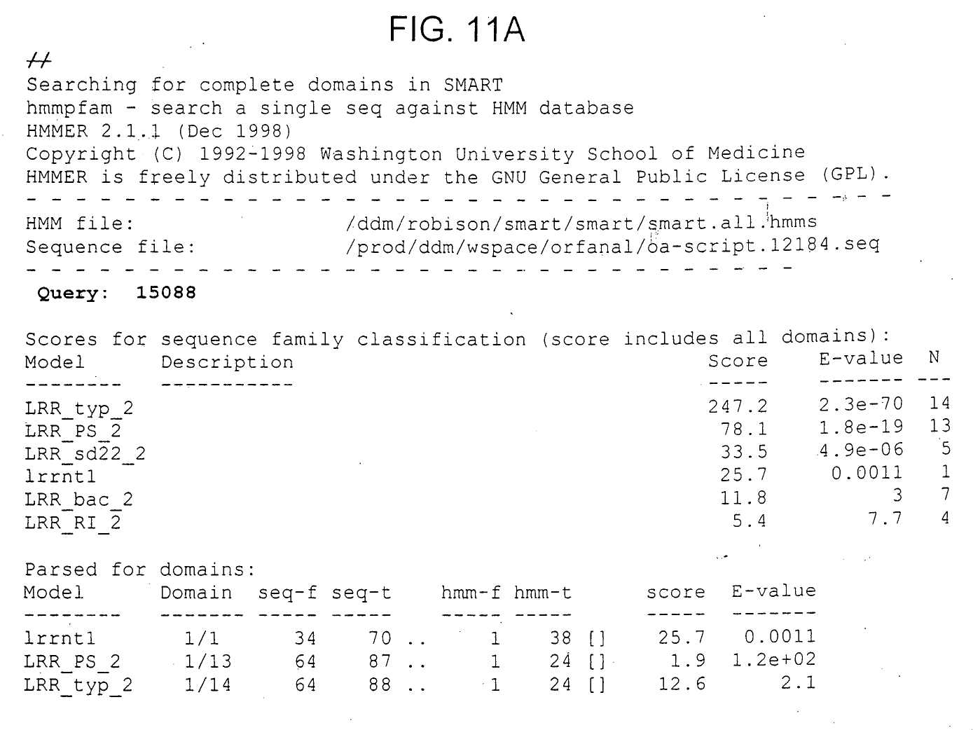

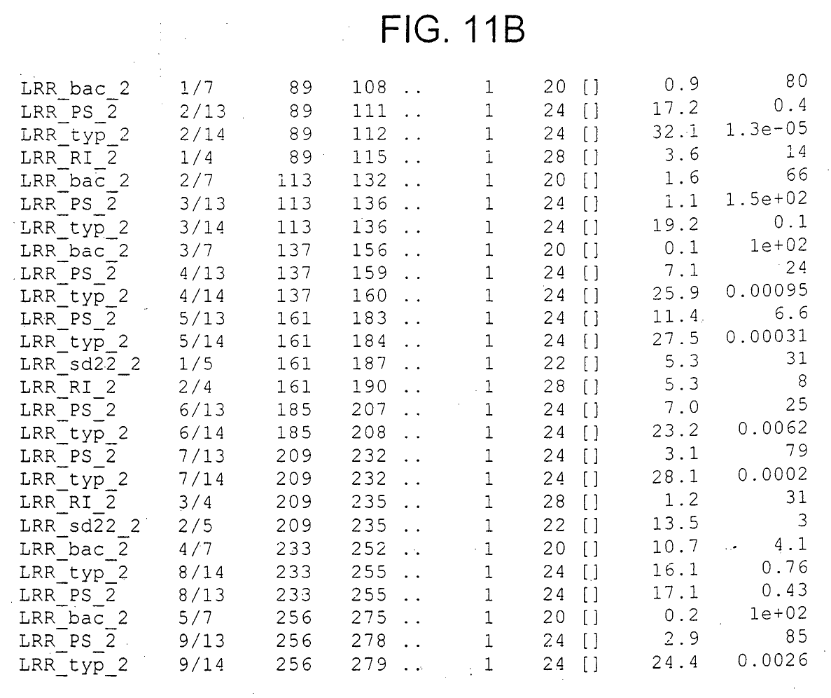

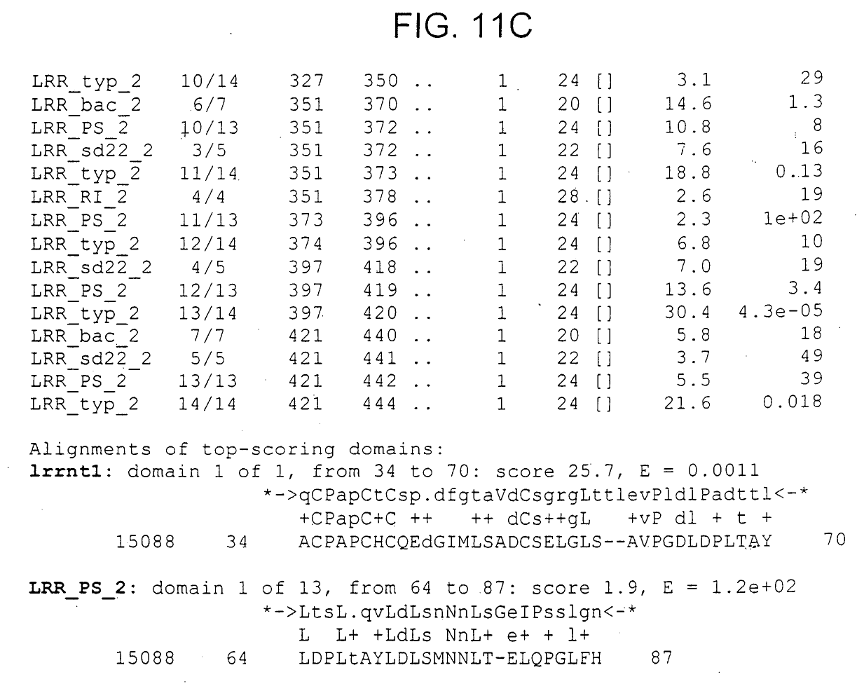

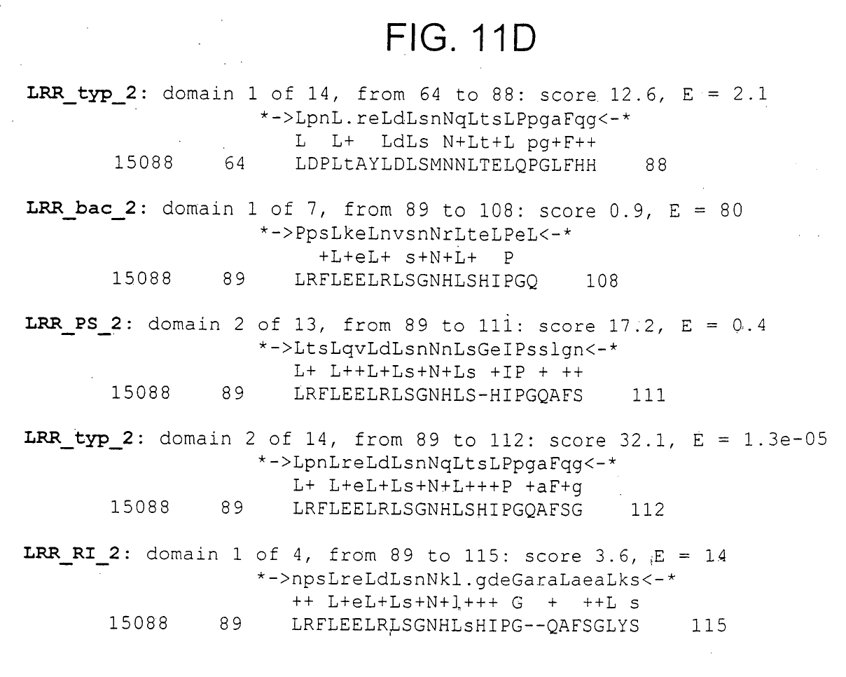

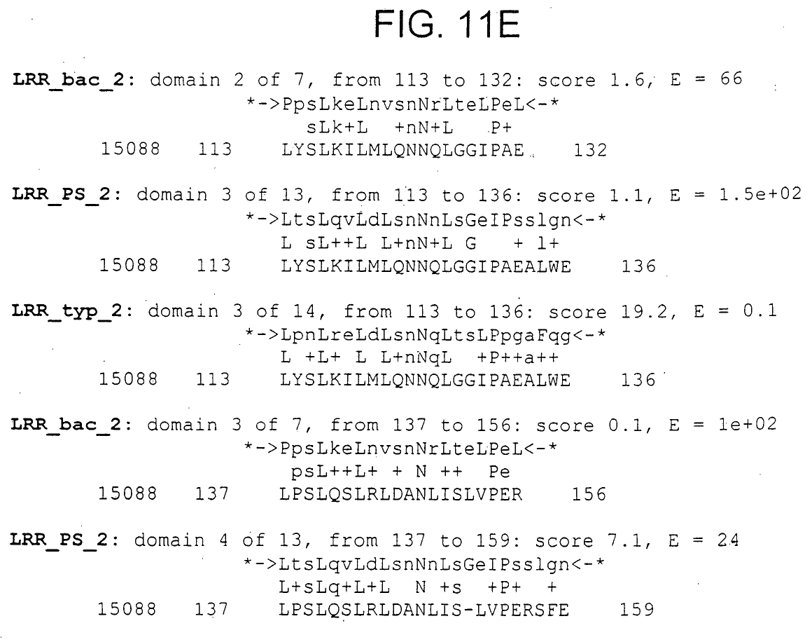

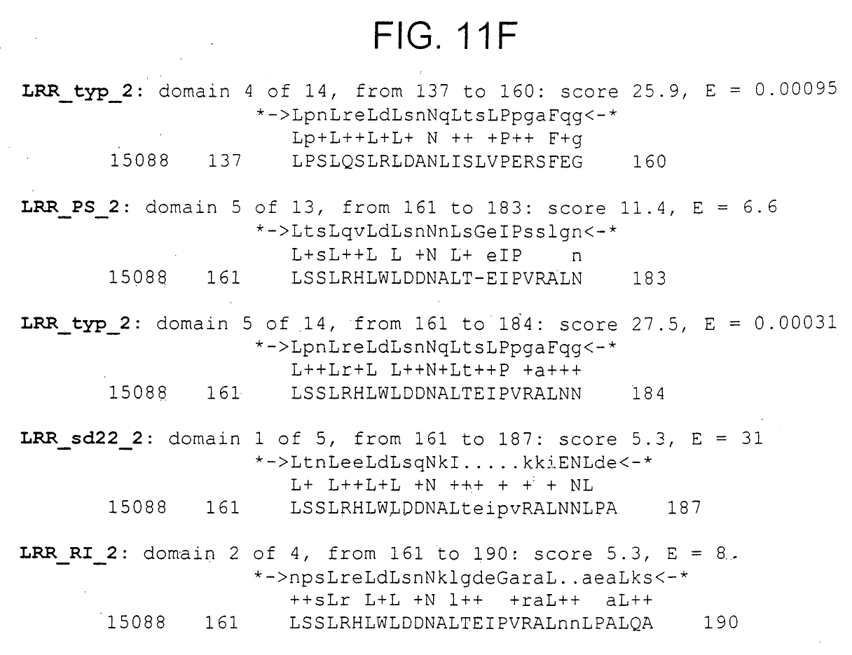

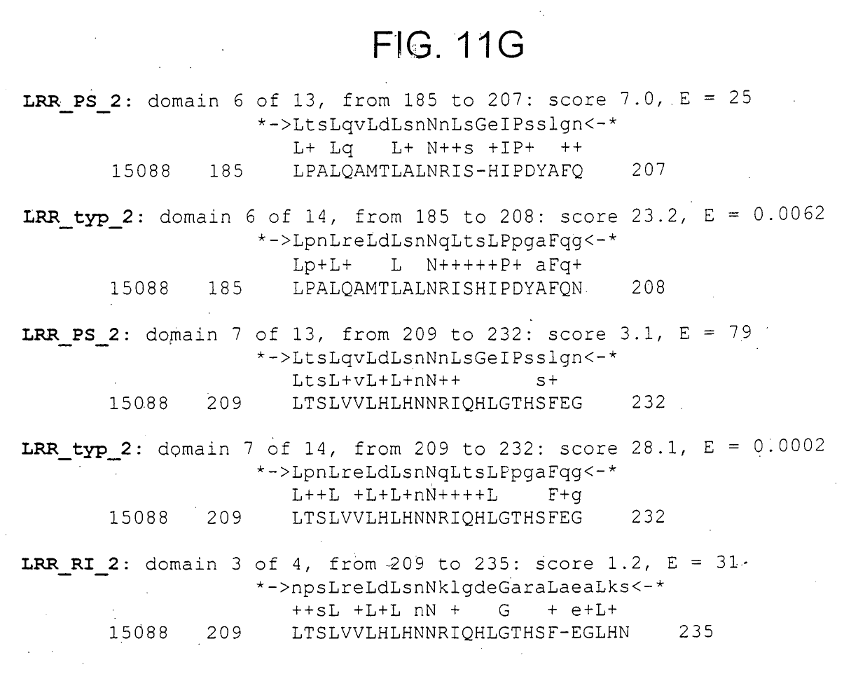

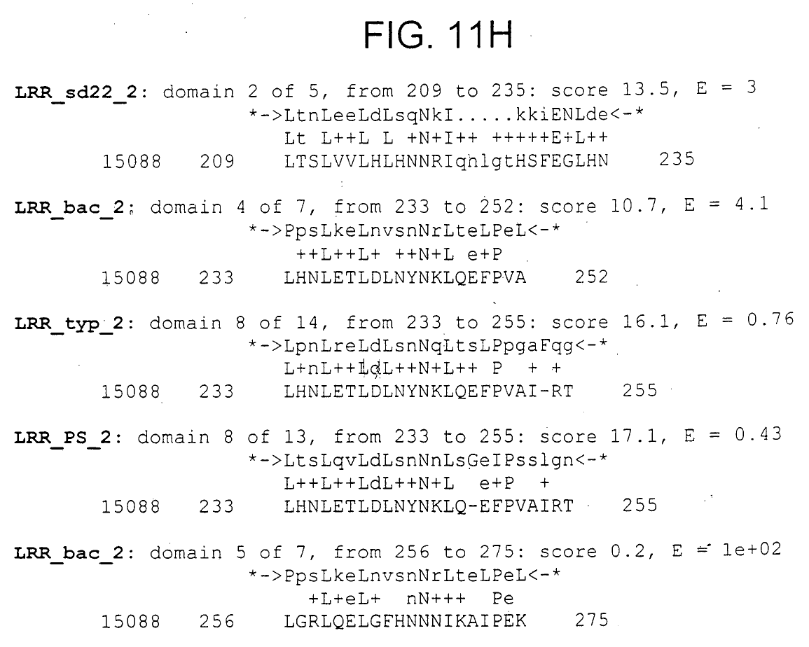

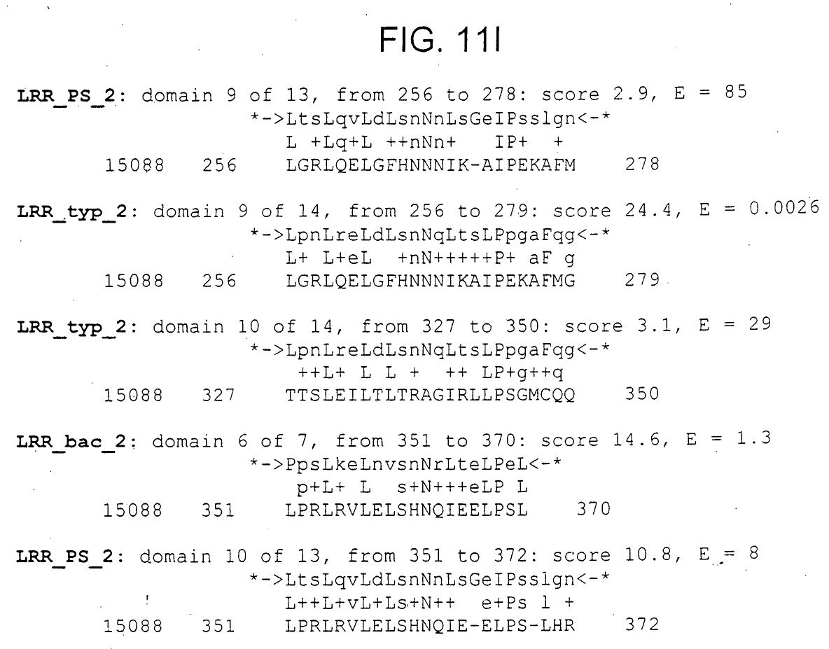

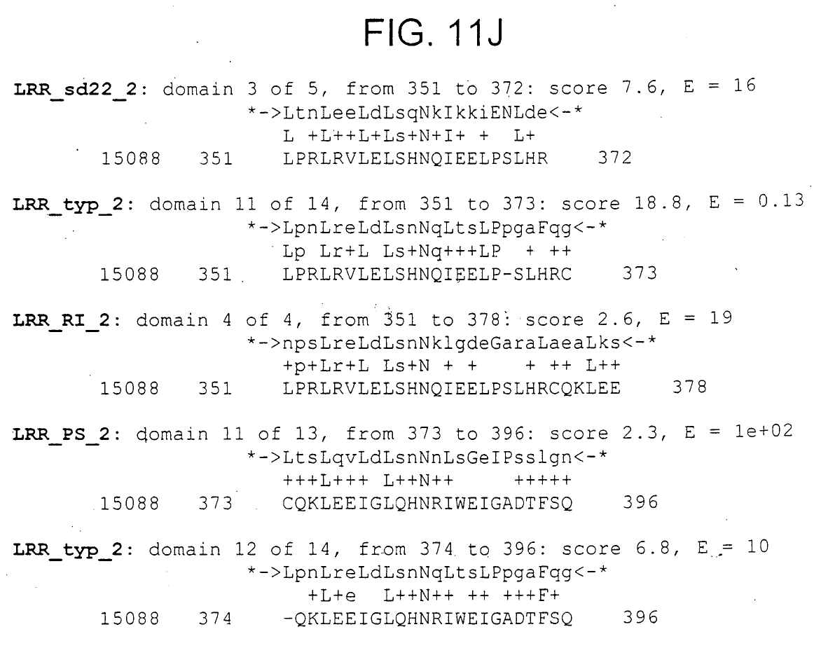

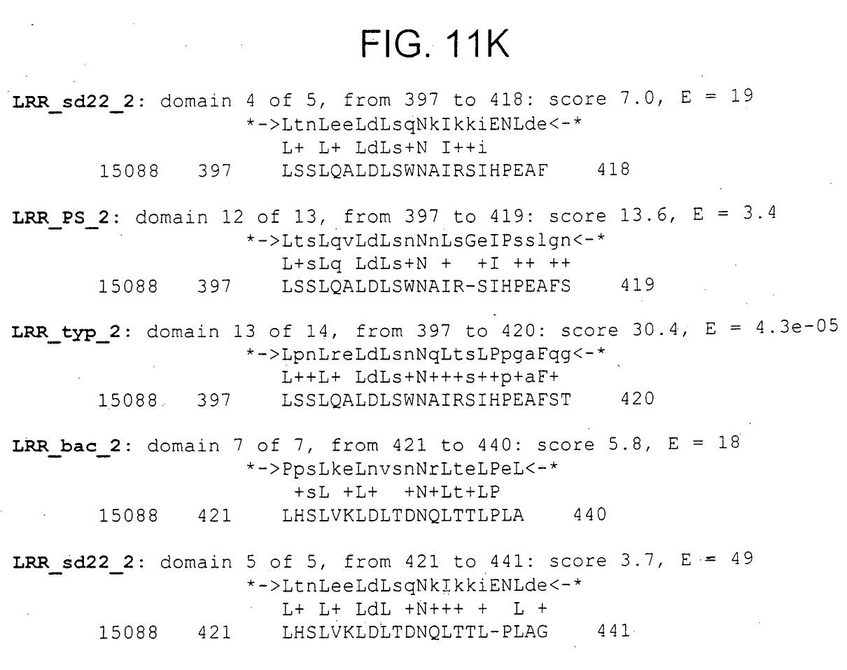

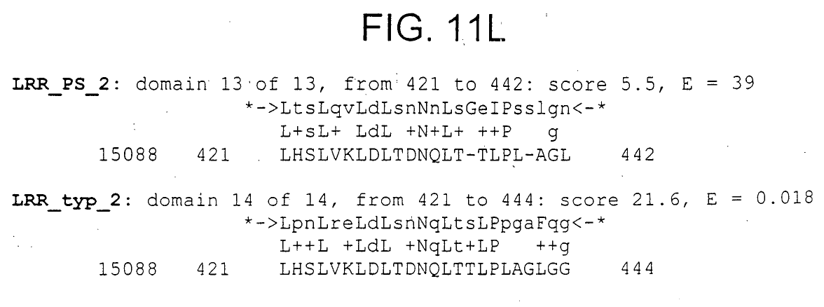

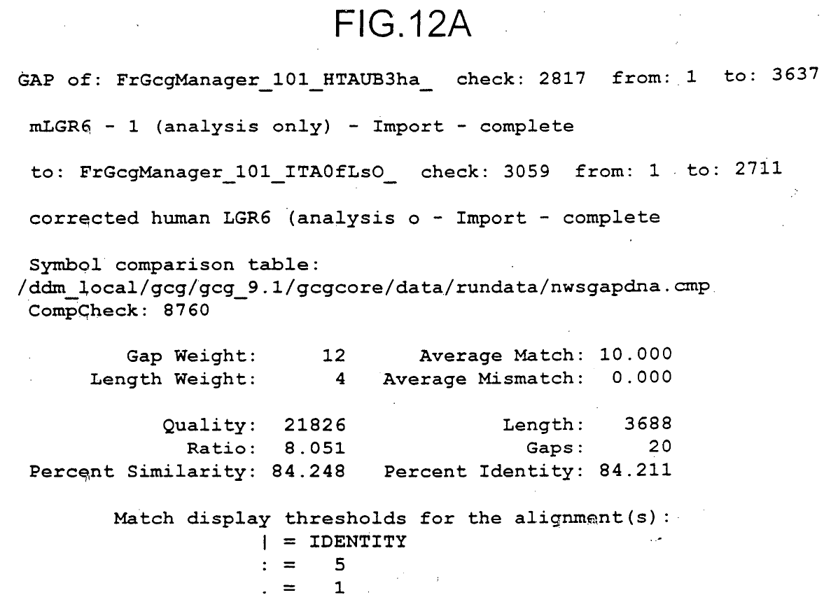

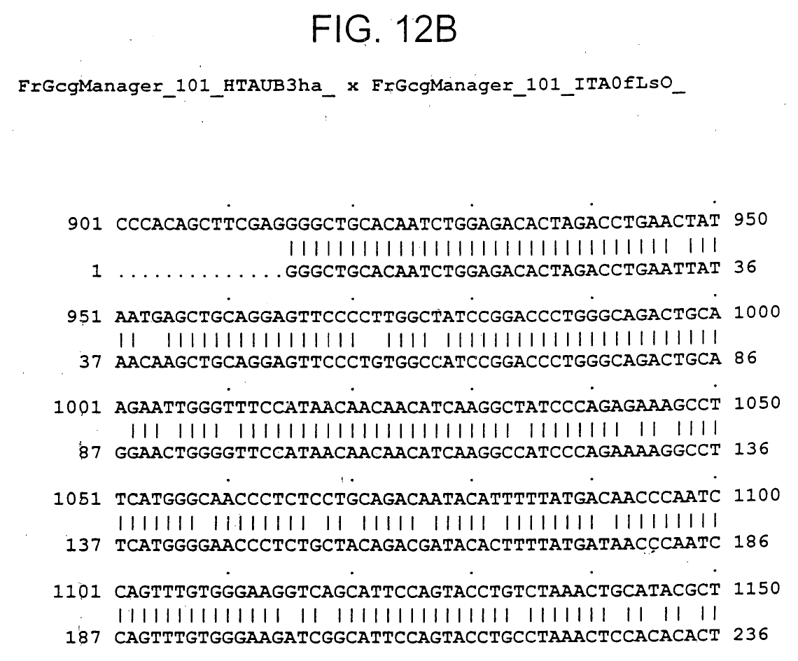

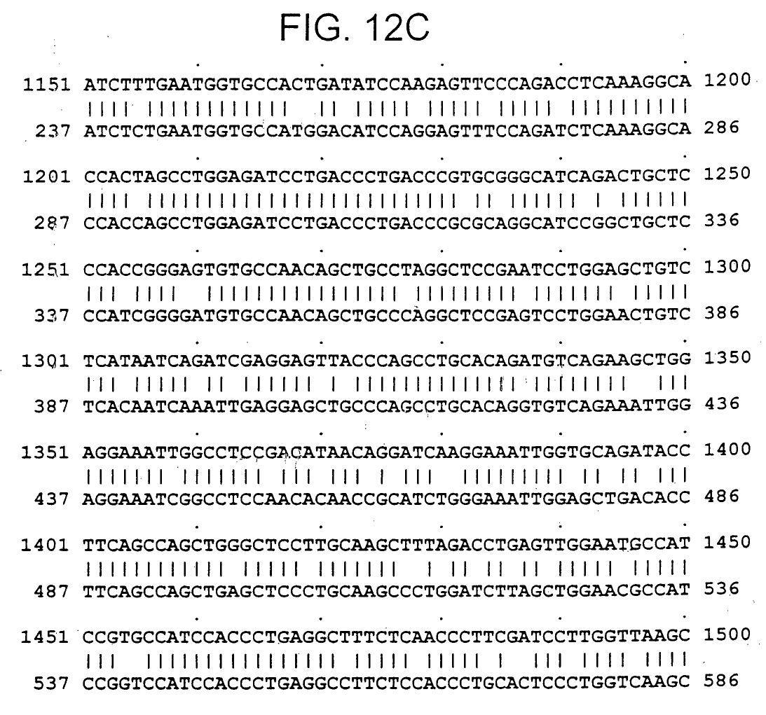

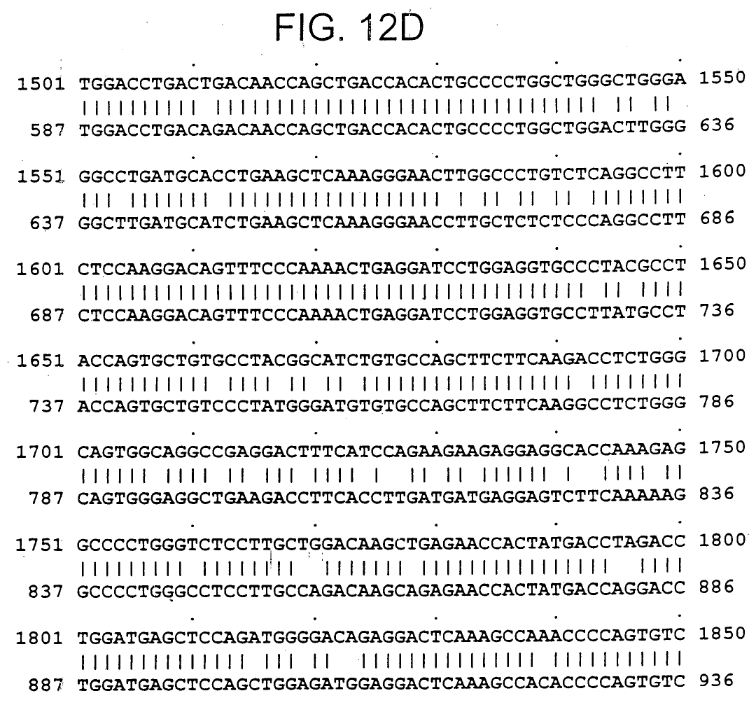

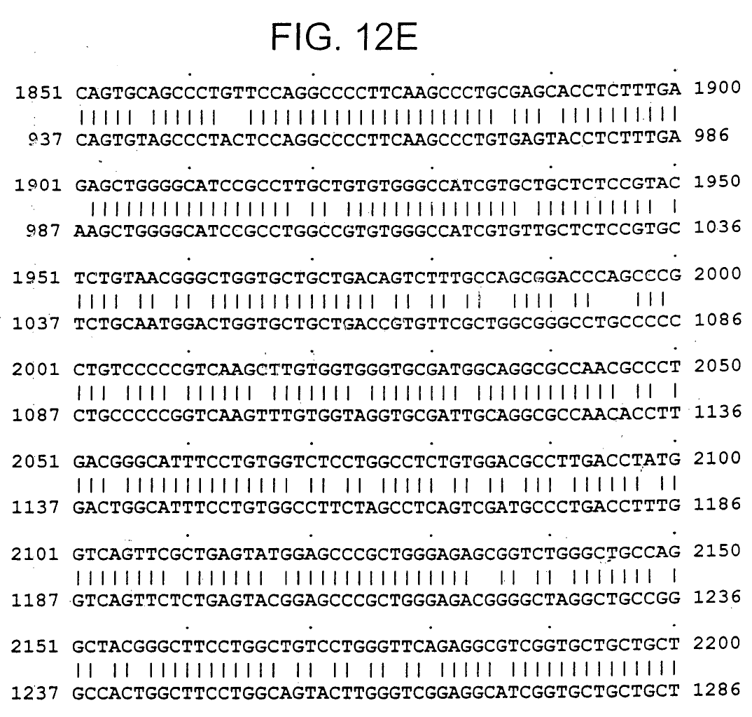

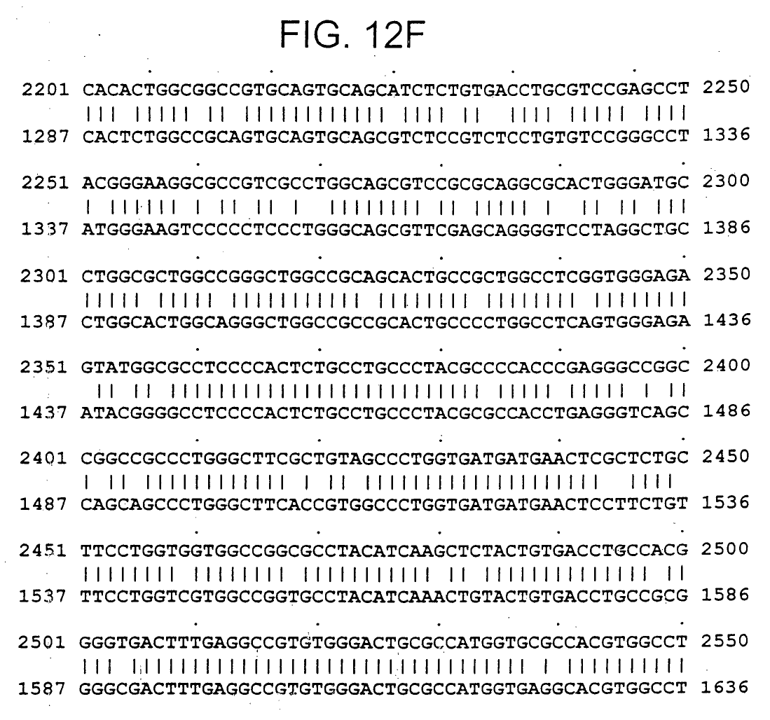

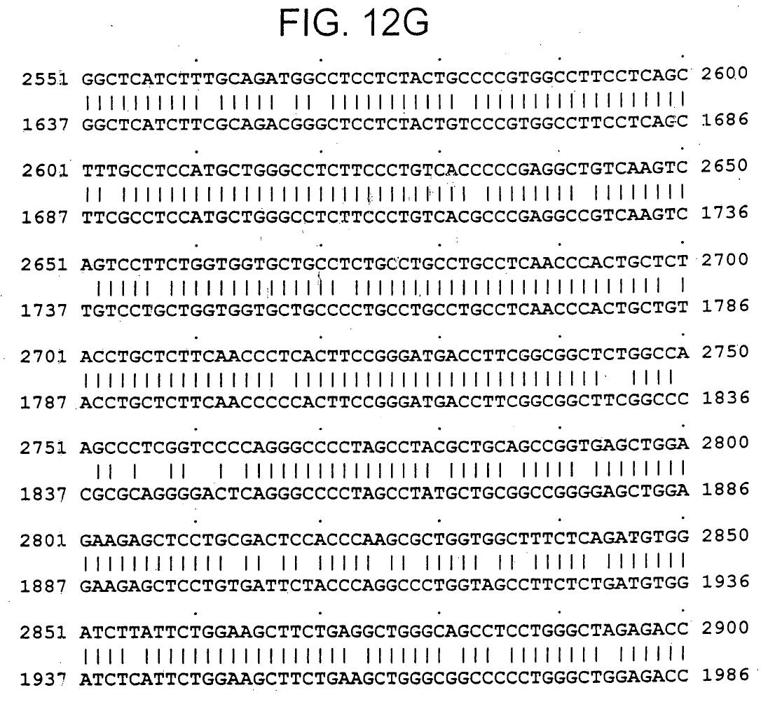

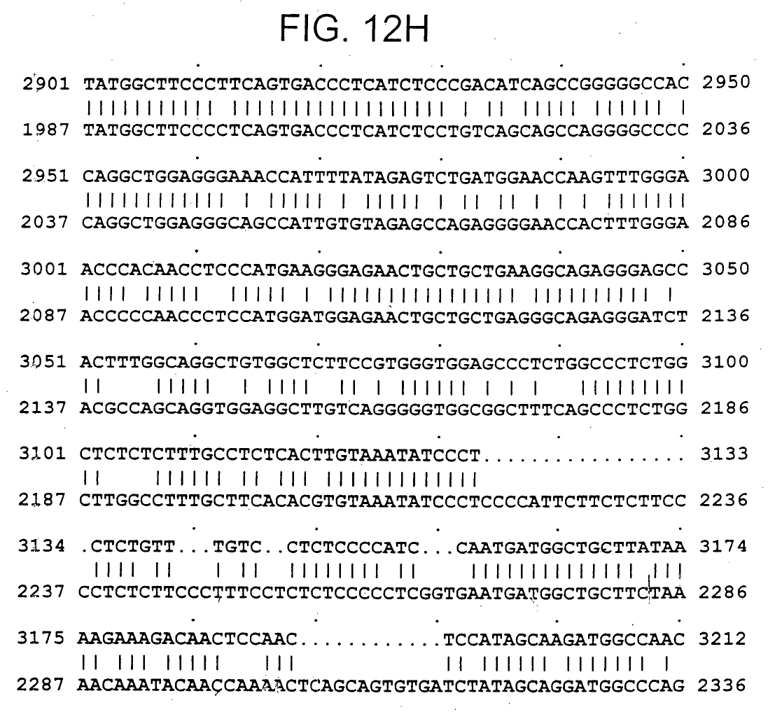

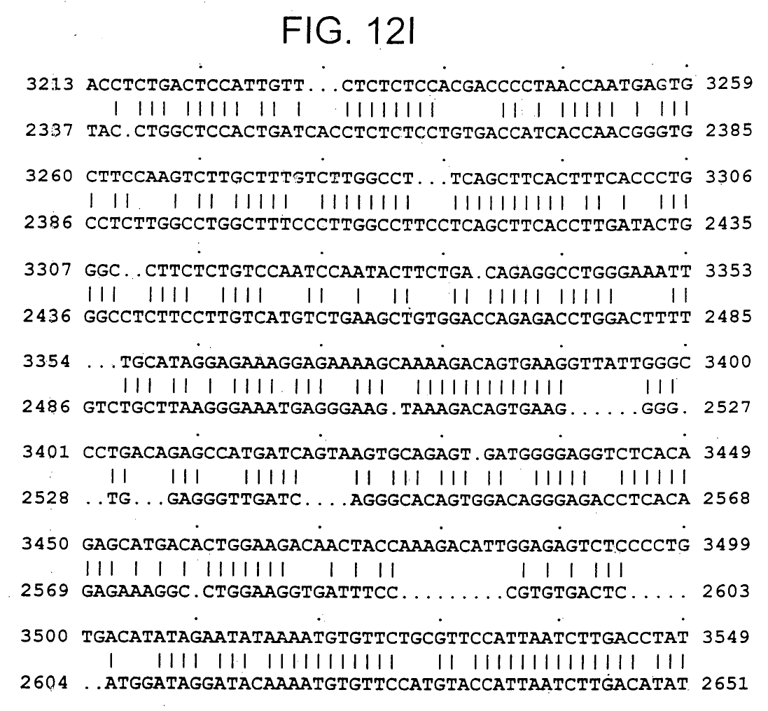

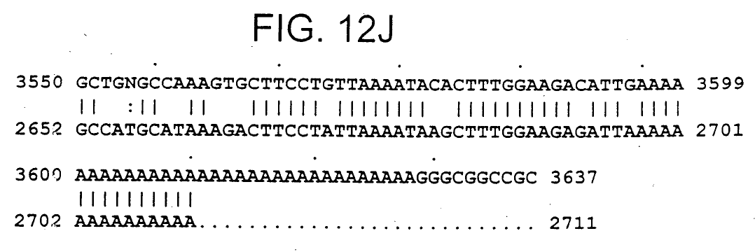

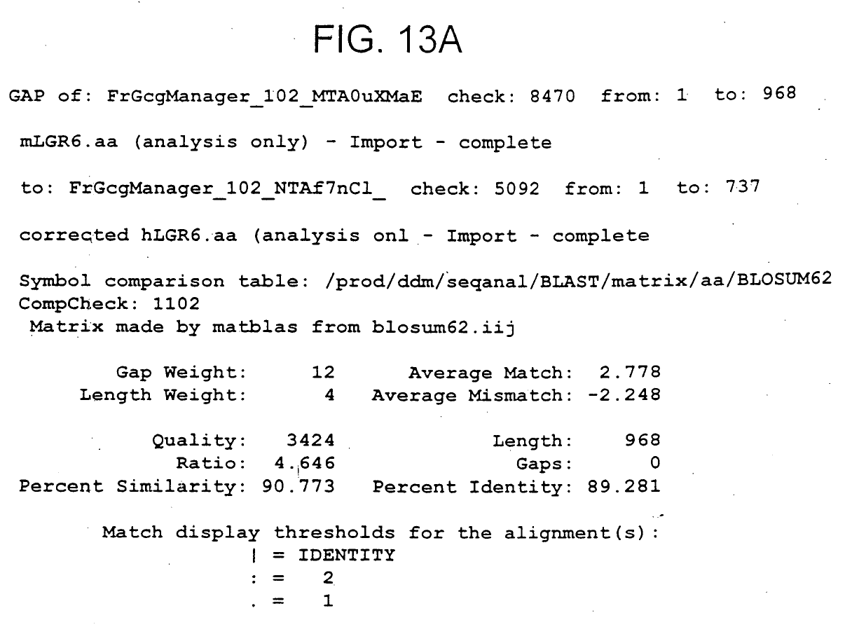

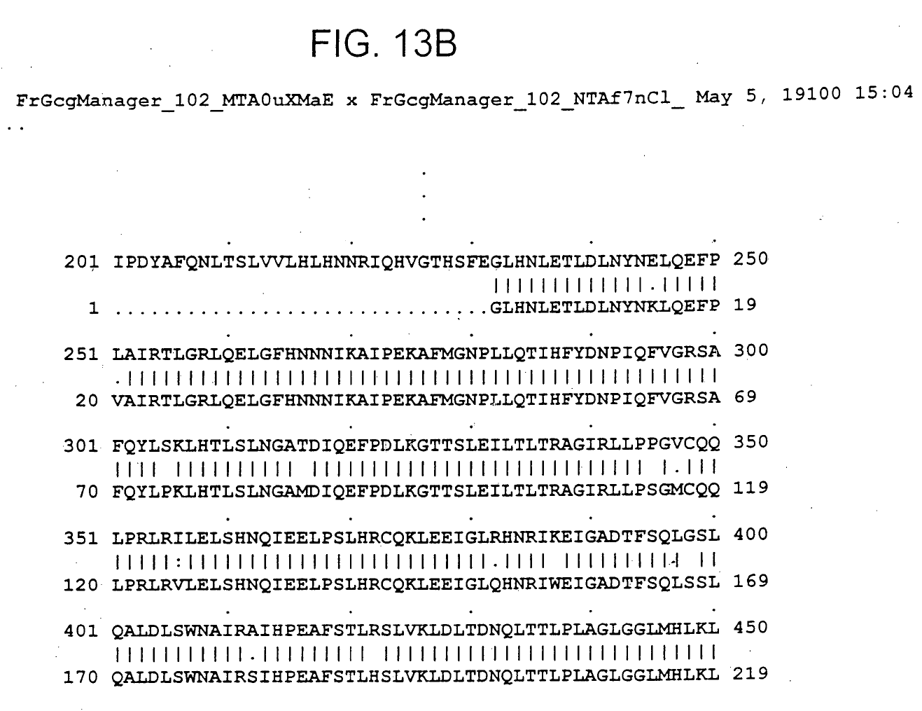

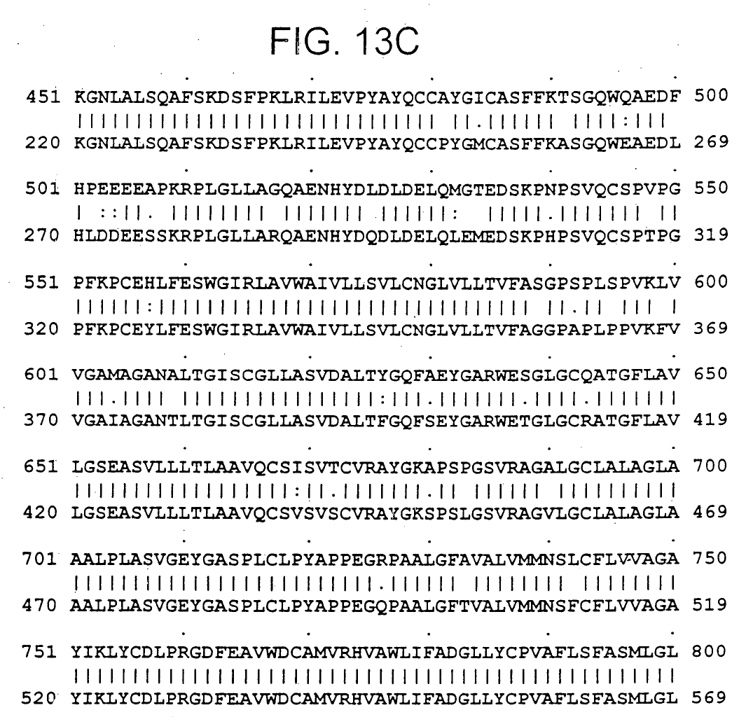

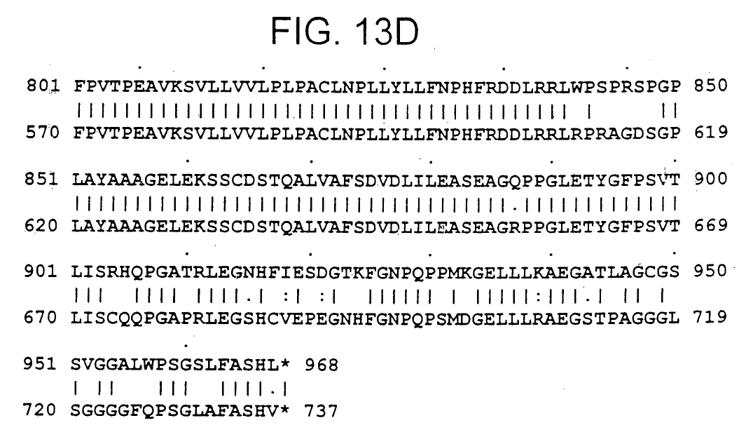

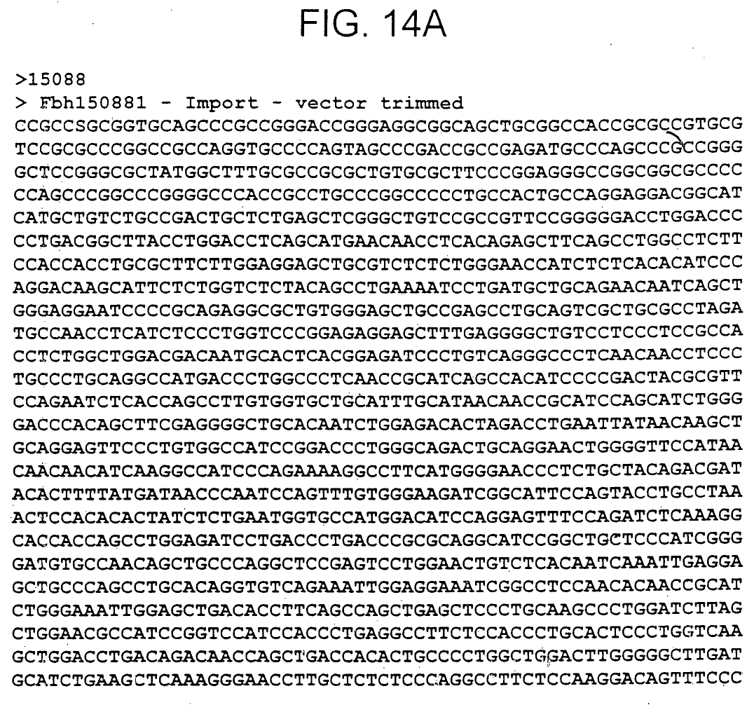

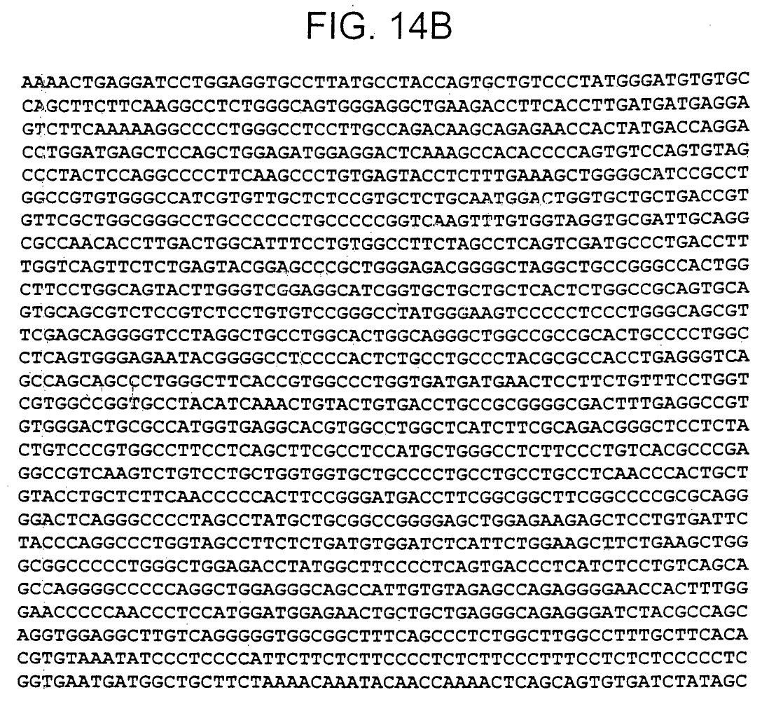

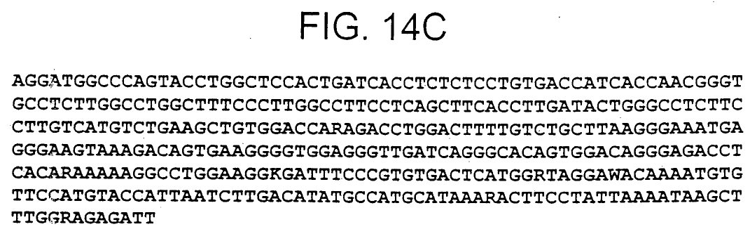

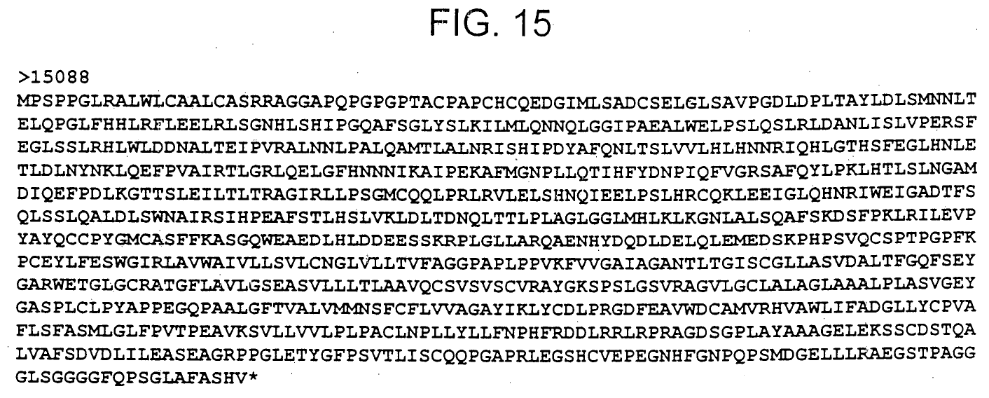

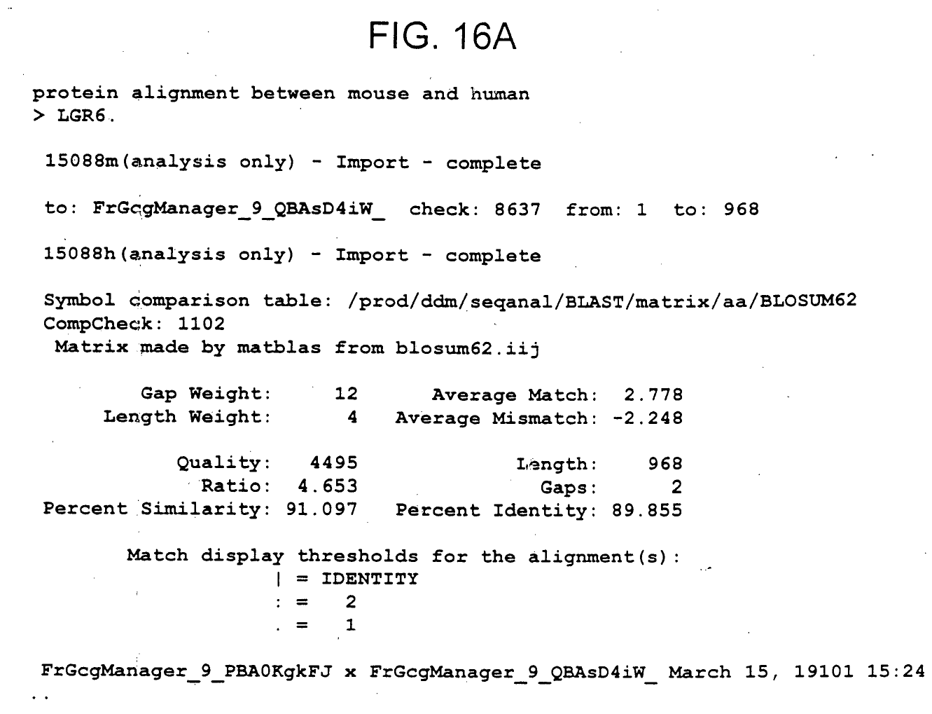

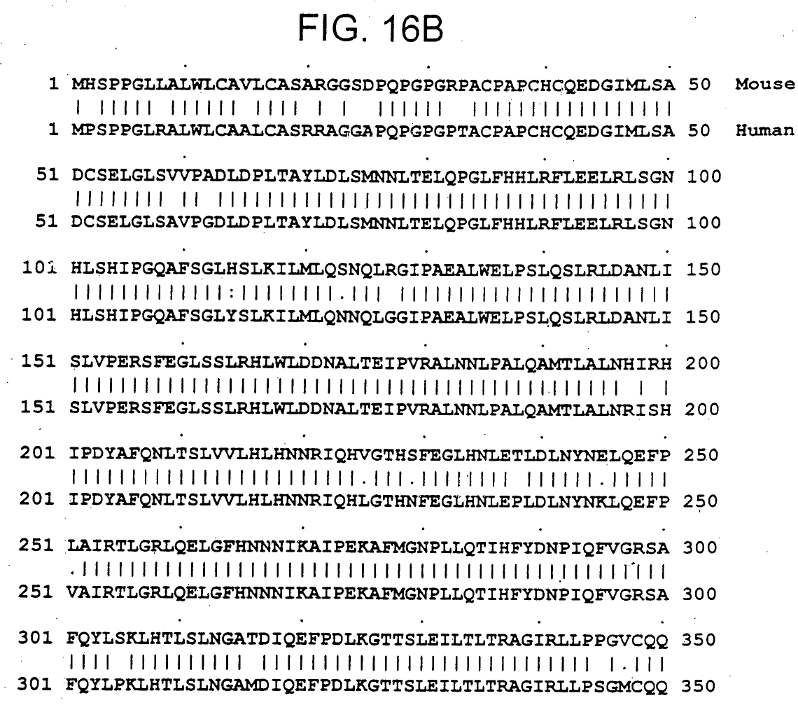

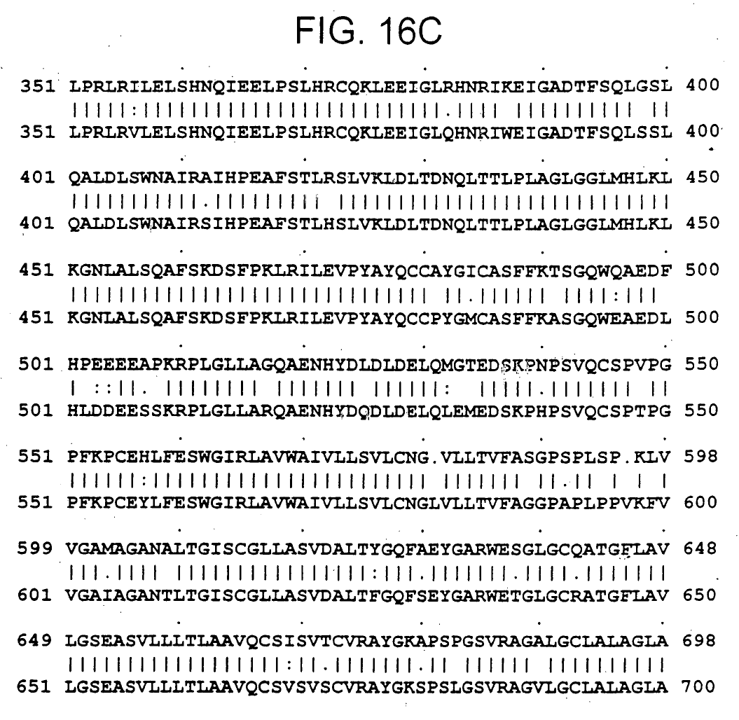

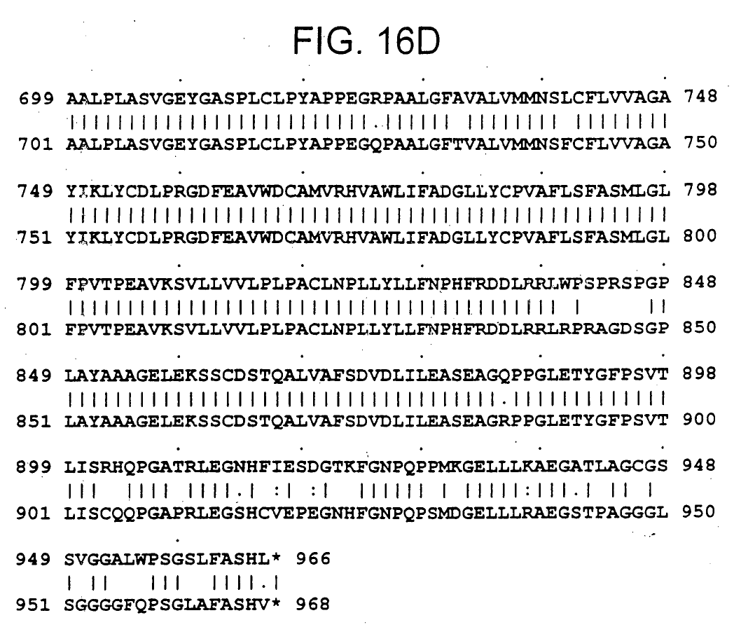

In one embodiment, the extracellular domain contains at least one, two, three, four, five, six, seven, eight, nine, ten, eleven, twelve, thirteen, fourteen, fifteen, and preferably, sixteen leucine-rich repeats. As used herein, a “leucine-rich repeat” (also referred to herein as “LRR”) refers to short protein modules characterized by a periodic distribution of hydrophobic amino acids, especially leucine residues, separated by more hydrophilic residues (Buchanan, S. and Gay, N. J. (1996) Prog. Biophys. Molec. Biol. Vol. 65 (No. ½): 144; Kobe, B. and Deisenhofer, J. (1994) Trends in Biochem Sci.: 415-421, the contents of which are incorporated herein by reference). LRRs are distinguished by a consensus sequence of about 20-30, preferably, 24 amino acids in length. As shown in FIG. 3, the LRR consensus sequence preferably contains leucines or other aliphatic residues at positions 2, 5, 7, 12, 16, 21 and 24, and asparagine, cysteine or threonine at position 10. Preferred LRRs contain exclusively asparagine at position 10, however, a cysteine residue may be substituted in this position (FIG. 3). Consensus sequences derived from LRRs in individual proteins often contain additional conserved residues in positions other than those mentioned above. For example, aliphatic and aromatic amino acids, sometimes glycines and prolines can also be found. The hydrophobic consensus residues in the carboxy-terminal parts of the repeats are commonly spaced by 3, 4, or 7 residues. Leucine-rich repeats are usually present in tandem, and the number of LRR ranges from one to about 30 repeats.

As used herein, the term “leucine rich repeat” includes a protein domain having an amino acid sequence of about 10-30 amino acid residues and having a bit score for the alignment of the sequence to the LRR domain (HMM) of at least about 5. Preferably, a LRR domain includes at least about 15-28, more preferably about 20-26 amino acid residues, or 22-24 amino acid residues, and has a bit score for the alignment of the sequence to the LRR domain (HMM) of at least about 8, 10, 16, 18, 19, 23, 25 or greater. The LRR domain (HMM) has been assigned the PFAM Accession PF00560 (http://genome.wustl.edu/Pfam/.html). To identify the presence of a LRR domain in a LGR6 protein, and make the determination that a protein of interest has a particular profile, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF00560 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci USA 84:4355-4358; Krogh et al. (1994) J. Mol. Biol. 235:1501-1531; and Stultz et al. (1993) Protein Sci. 2:305-314, the contents of which are incorporated herein by reference.

In one embodiment, the LRR corresponds to a β-α structural unit, consisting of a short β-strand and an α-helix approximately parallel to each other. The structural units are arranged so that the β-strands and the helices are parallel to a common axis, resulting in a nonglobular, horseshoe-shaped molecule with a parallel β-sheet lining in the inner circumference of the horseshoe, and the helices flanking the circumference. Leucine-rich repeats are located at about amino acid residues 67 to 90, 91 to 114, 115 to 138, 139 to 162, 163 to 186, 187 to 210, 211 to 234, 235 to 257, 258 to 281, 282 to 305, 306 to 329, 330 to 352, 353 to 375, 376 to 398, 399 to 422 and 423 to 446 of SEQ ID NO:2 of SEQ ID NO:2, and at about amino acids 64 to 87 and 88 to 111 of SEQ ID NO:5. In addition, a search was performed against the HMM database resulting in the identification of LRR domains in the amino acid sequence of human LGR6 at about residues 4-26, 27-50, 51-74, 75-97, 98-121, 122-143, 144-167, 168-191, and 192-215 of SEQ ID NO:8. A search was also performed against the HMM database resulting in the identification of LRR domains in the amino acid sequence of the complete human LGR6 at about residues 67 to 90, 91 to 114, 115 to 138, 139 to 162, 163 to 186, 187 to 210, 211 to 234, 235 to 257, 258 to 281, 282 to 305, 306 to 329, 330 to 352, 353 to 375, 376 to 398, 399 to 422 and 423 to 446 of SEQ ID NO:11 (see FIGS. 10 and 11). The LRR domains identified in the amino acid sequence of human LGR6 of SEQ ID NO:8 correspond to amino acid residues 235 to 257, 258 to 281, 282 to 305, 306 to 329, 330 to 352, 353 to 375, 376 to 398, 399 to 422 and 423 to 446 of SEQ ID NO:11

Accordingly, LGR6 proteins having at least 50-60% identity, preferably about 60-70%, more preferably about 70-80%, or about 80-90% identity with a LRR domain of human or mouse LGR6 are within the scope of the invention.

Preferably, the leucine-rich repeat in the extracellular domain of an LGR6 protein mediates protein-protein interactions, signal transduction and/or cell adhesion. In one embodiment, the LRR domain is capable of interacting (e.g., binding to) a glycoprotein hormone. Exemplary glycoprotein hormones include gonadotropins (e.g., luteinizing hormone (LH), follicle-stimulating hormone (ESH), choriogonadotropin (CG) and thyroid-stimulating hormone (TSH)). Upon binding of an extracellular protein to the LRR, an intracellular signal transduction pathway (e.g., adenylate cyclase pathway or PI turnover pathway) is activated. For example, the Gs-cAMP-protein kinase A pathway can be activated (Ji, T. H. et al. (1997) Recent Prog. Horm. Res. 52:431-453; Dufau, M. L. (1998) Annu. Rev. Physiol. 60: 461-496; Kohn, L. D. (1995) Vitam. Horm. 50: 287-384; Simoni, M. et al. (1997) Endocr. Rev. 18: 739-773). Alternatively, or in addition to the ligand binding role, the LRRs may mediate receptor dimerization or oligomerization. Such aggregation has been shown, for a number of receptor types, to correlate with their activation. Examples of the receptors that are activated upon dimerization include receptor tyrosine kinases (RTK) and serine/threonine kinases.

In one embodiment, the LGR6 proteins of the present invention contain at least one RGD cell attachment site. As used herein, the term “RGD cell attachment site” refers to a cell adhesion sequence consisting of amino acids Arg-Gly-Asp typically found in extracellular matrix proteins such as collagens, laminin and fibronectin, among others (reviewed in Ruoslahti, E. (1996) Annu. Rev. Cell Dev. Biol. 12:697-715). Preferably, the RGD cell attachment site is located in the extracellular domain of an LGR6 protein and interacts (e.g., binds to) a cell surface receptor, such as an integrin receptor. As used herein, the term “integrin” refers to a family of receptors comprising αβ heterodimers that mediate cell attachment to extracellular matrices and cell-cell adhesion events. The α subunits vary in size between 120 and 180 kd and are each noncovalently associated with αβ subunit (90-110 kd) (reviewed by Hynes (1992) Cell 69:11-25). Most integrins are expressed in a wide variety of cells, and most cells express several integrins. There are at least 8 known P subunits and 14 known α subunits. The majority of the integrin ligands are extracellular matrix proteins involved in substratum cell adhesion such as collagens, laminin, fibronectin among others. The RGD cell attachment site is located at about amino acid residues 760-762 of SEQ ID NO:2, at amino acids 425-427 of SEQ ID NO:5, at amino acid residues 529-531 of SEQ ID NO:8 and at amino acid residues 760-762 of SEQ ID NO:11.

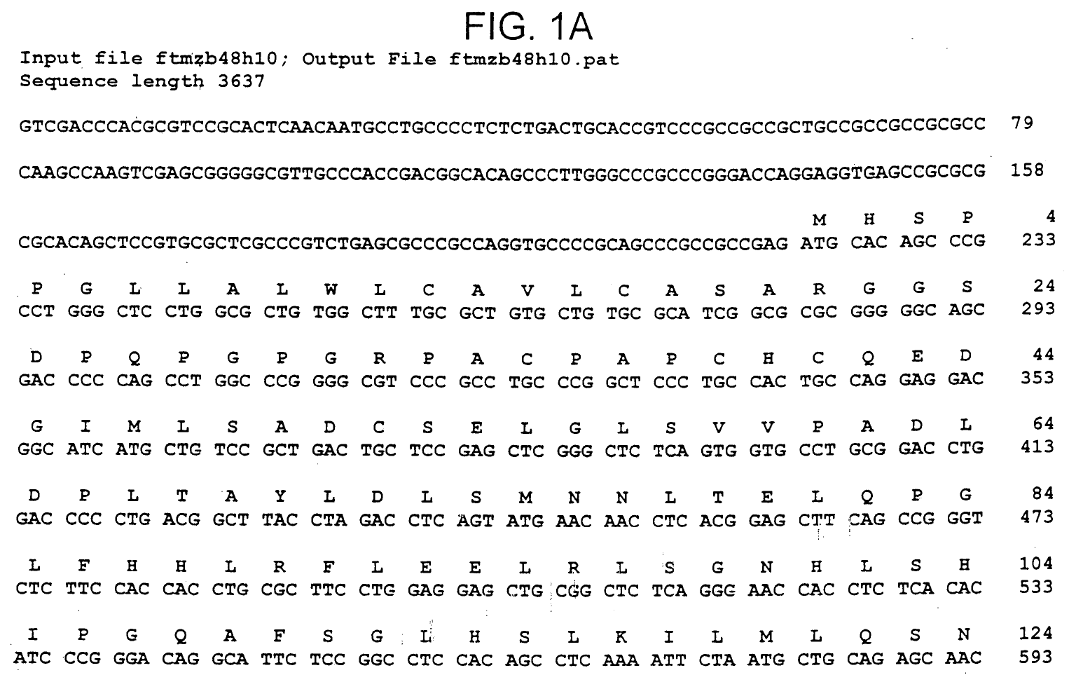

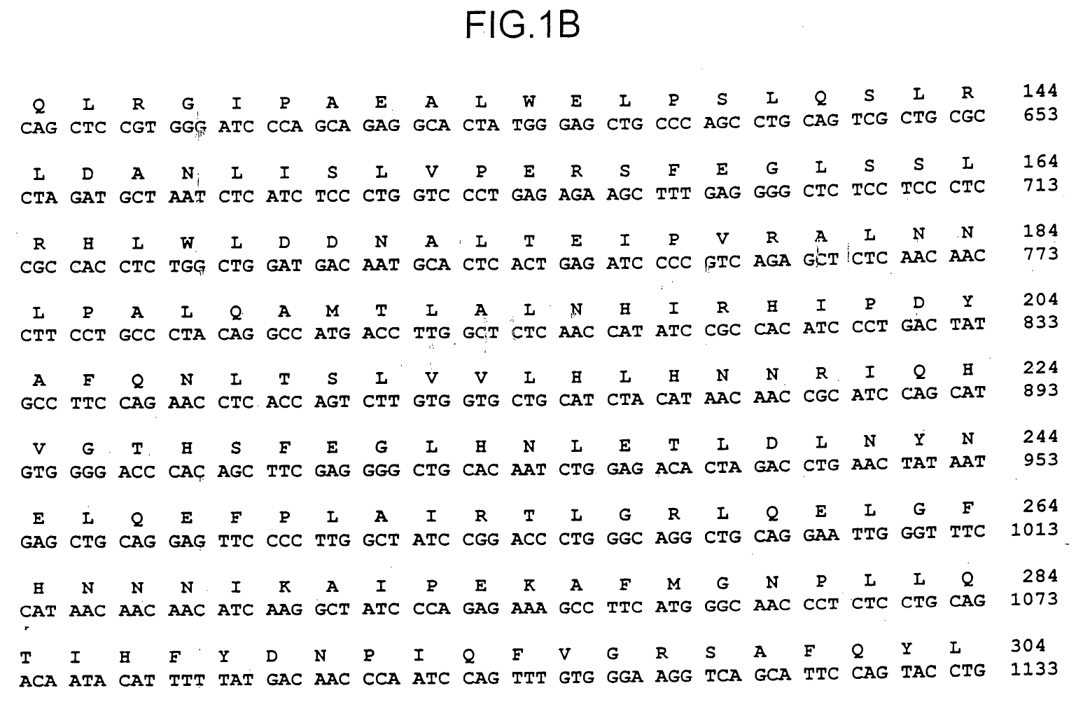

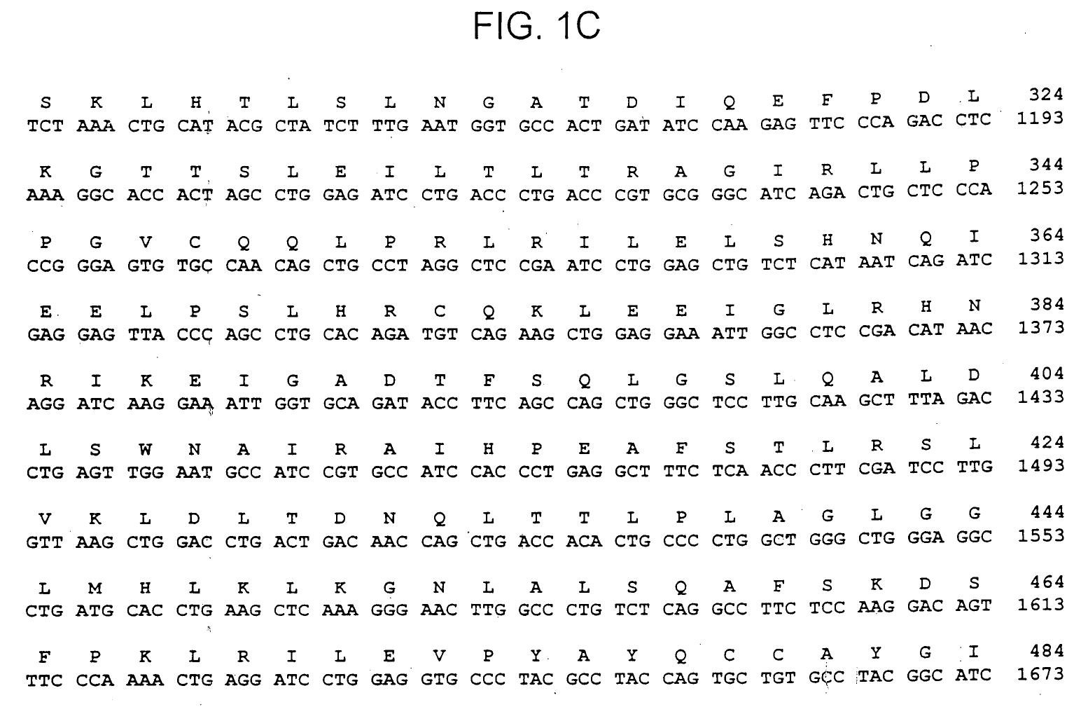

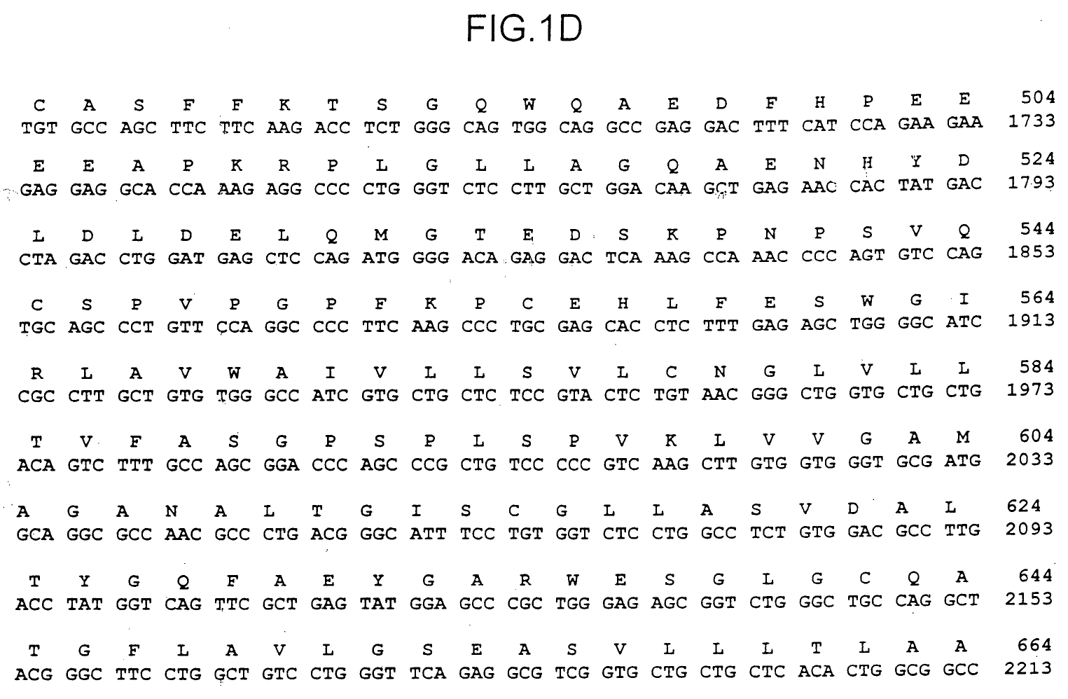

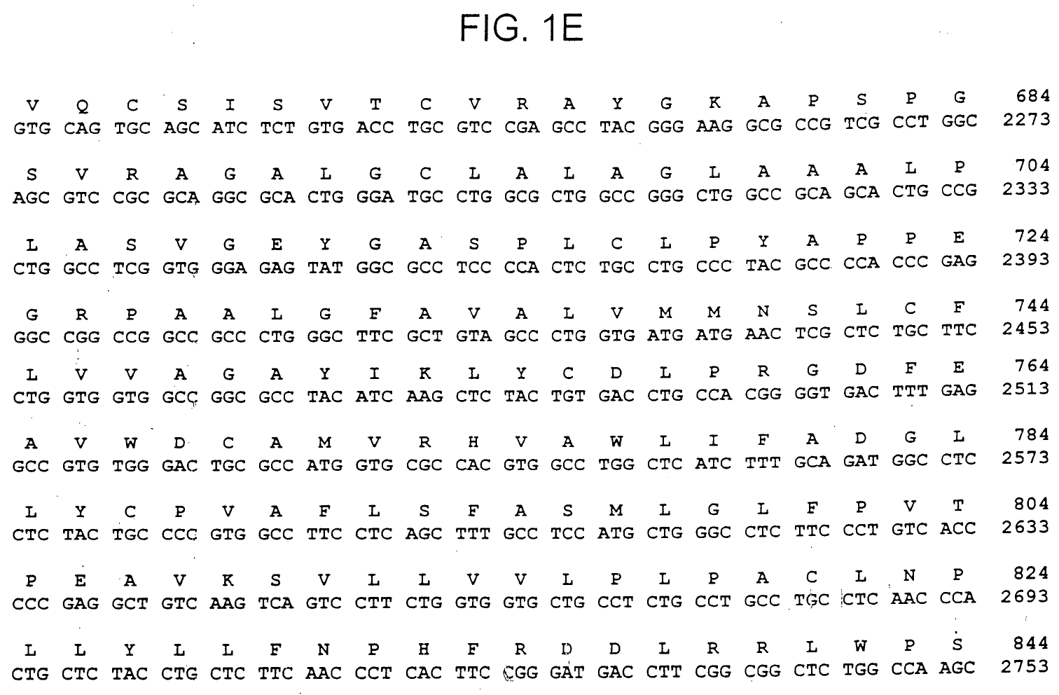

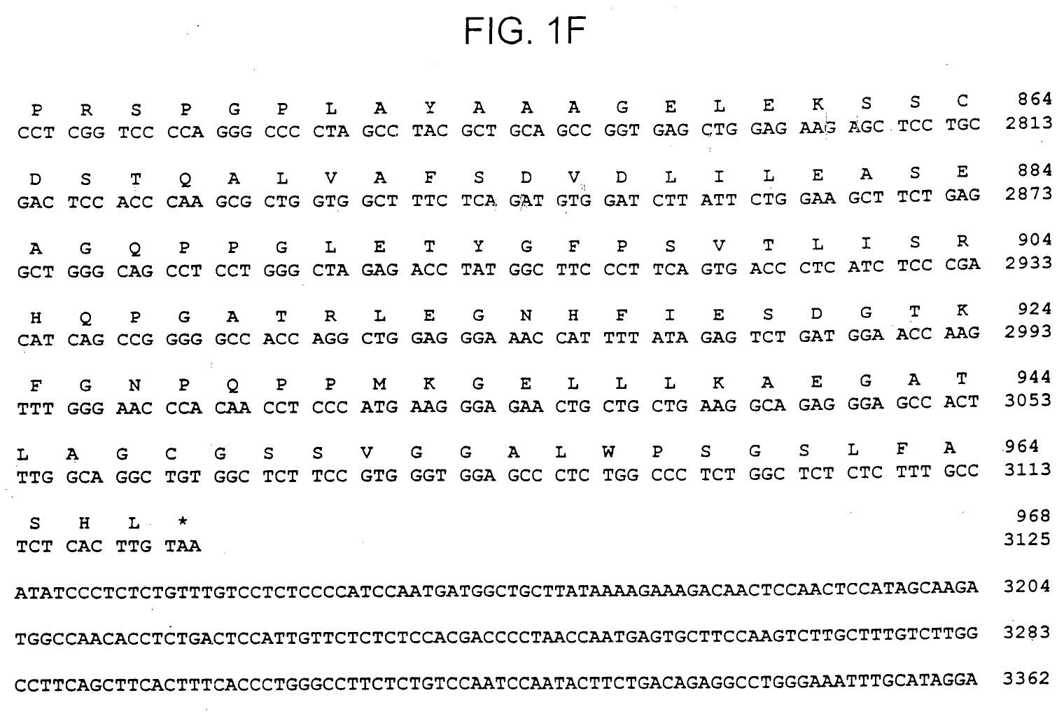

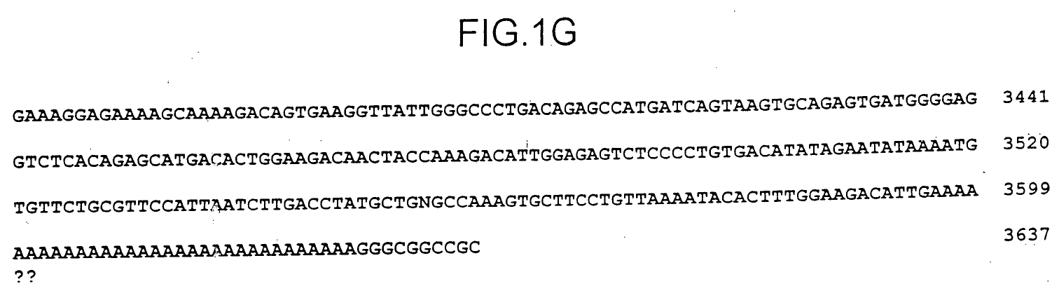

In another embodiment, the LGR6 proteins of the present invention contain at least one, two, three, four, five, six, or preferably, seven transmembrane domains. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, htto://pfam.wustl.edu/cgi-bin/getdesc?name=7tm-1, and Zagotta W. N. et al, (1996) Annual Rev. Neuronsci. 19: 235-63, the contents of which are incorporated herein by reference. Amino acid residues 564-590, 598-620, 645-669, 684-704, 731-751, 773-798 and 812-834 of SEQ ID NO:2 comprise transmembrane domains (see FIG. 1). Amino acid residues 230-256, 264-286, 311-336, 350-370, 397-417, 440-464 and 478-500 of SEQ ID NO:5 comprise transmembrane domains (see FIG. 5). Amino acid residues 333-359, 367-389, 414-439, 453-473, 500-520, 543-567 and 581-603 of SEQ ID NO:8 comprise transmembrane domains (see FIG. 8). Amino acid residues 566-590, 599-621, 646-665, 688-709, 728-752 and 777-801 of SEQ ID NO:11 comprise transmembrane domains (see FIG. 15).

In another embodiment, an LGR6 includes at least one “7 transmembrane receptor profile” in the protein or corresponding nucleic acid molecule. As used herein, the term “7 transmembrane receptor profile” includes an amino acid sequence having at least about 10-300, preferably about 15-200, more preferably about 20-100 amino acid residues, or at least about 22-100 amino acids in length and having a bit score for the alignment of the sequence to the 7tm_{—}1 family Hidden Markov Model (HMM) of at least 1, preferably 3, more preferably 5-10, preferably 20-30, more preferably 2240, more preferably 40-50, 50-75, 75-100, 100-200 or greater. The 7tm_{—}1 family HMM has been assigned the PFAM Accession PF00001 (http://genome.wustl.edu/Pfam/WWdata/7_tm_{—}1.html).

To identify the presence of a 7 transmembrane receptor profile in an LGR6, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF00001 and score of 15 is the default threshold score for determining a hit. A search was performed against the HMM database resulting in the identification of 7 tm_{—}1 domains in the amino acid sequence of human LGR6 at about residues 404-431 and 553-596 of SEQ ID NO:8. A search was was also performed against the HMM database resulting in the identification of 7 tm_{—}1 domains in the amino acid sequence of human LGR6 at about and amino acids 635 to 662 and 784 to 827 of SEQ ID NO:11 (see FIG. 10). The 7 tm_{—}1 domains in the amino acid sequence of human LGR6 at about amino acids 635 to 662 and 784 to 827 of SEQ ID NO:11 correspond to the 7 tm_{—}1 domains in the amino acid sequence of human LGR6 at about residues 404-431 and 553-596 of SEQ ID NO:8. Alternatively, the seven transmembrane domain can be predicted based on stretches of hydrophobic amino acids forming α-helices (SOUSI server). For example, using a SOUSI server, a 7 TM receptor profile was identified in the amino acid sequence of SEQ ID NO:2, SEQ ID NO:5 (e.g., amino acids 812-834 of SEQ ID NO:2, amino acids 478-500 of SEQ ID NO:5). Accordingly, LGR6 proteins having at least 50-60% homology, preferably about 60-70%, more preferably about 70-80%, or about 80-90% homology with the 7 transmembrane receptor profile of human or mouse LGR6 are within the scope of the invention.

In another embodiment, an LGR6 protein includes at least one extracellular loop. As defined herein, the term “loop” includes an amino acid sequence having a length of at least about 4, preferably about 5-10, preferably about 10-20, and more preferably about 20-30, 30-40, 40-50, 50-60, 60-70, 70-80, 80-90, 90-100, or 100-150 amino acid residues, and has an amino acid sequence that connects two transmembrane domains within a protein or polypeptide. Accordingly, the N-terminal amino acid of a loop is adjacent to a C-terminal amino acid of a transmembrane domain in a naturally-occurring LGR6 or LGR6-like molecule, and the C-terminal amino acid of a loop is adjacent to an N-terminal amino acid of a transmembrane domain in a naturally-occurring LGR6 or LGR6-like molecule. As used herein, an “extracellular loop” includes an amino acid sequence located outside of a cell, or extracellularly. For example, an extracellular loop can be found at about amino acids 621-644, 705-730 and 799-811 of SEQ ID NO:2, at amino acids 287-310, 371-396 and 465-477 of SEQ ID NO:5, or at amino acids 390-413, 474-499 and 568-580 of SEQ ID NO:8.

In another embodiment, an LGR6 protein include at least one cytoplasmic loop, also referred to herein as a cytoplasmic domain. As used herein, a “cytoplasmic loop” includes an amino acid sequence located within a cell or within the cytoplasm of a cell. For example, a cytoplasmic loop is found at about amino acids 591-597, 670-683 and 752-772 of SEQ ID NO:2. In other embodiments, the cytoplasmic loop is found at about amino acids 257-263, 337-349 and 418-439 of SEQ ID NO:5. In addition, a cytoplasmic loop is found at about amino acids 360-366, 440-452 and 521-542 of SEQ ID NO:8.

In another embodiment of the invention, an LGR6 is identified based on the presence of a “C-terminal cytoplasmic domain”, also referred to herein as a C-terminal cytoplasmic tail, in the sequence of the protein. As used herein, a “C-terminal cytoplasmic domain” includes an amino acid sequence having a length of at least about 10, preferably about 10-25, more preferably about 25-50, more preferably about 50-75, even more preferably about 75-100, 100-133, 133-150, 150-200, 200-250, 250-300, 300-400, 400-500, or 500-600 amino acid resudues and is located within a cell or within the cytoplasm of a cell. Accordingly, the N-terminal amino acid residue of a “C-terminal cytoplasmic domain” is adjacent to a C-terminal amino acid residue of a transmembrane domain in a naturally-occurring LGR6 or LGR6-like protein. For example, a C-terminal cytoplasmic domain is found at about amino acid residues 835-968 of SEQ ID NO:2, at amino acid residues 501-633 of SEQ ID NO:5, or at amino acid residues 604-736 of SEQ ID NO:8.

In yet another embodiment, the LGR6 molecule can further include a signal sequence. As used herein, a “signal sequence” refers to a peptide of about 20-30 amino acid residues in length which occurs at the N-terminus of secretory and integral membrane proteins and which contains a majority of hydrophobic amino acid residues. For example, a signal sequence contains at least about 15-45 amino acid residues, preferably about 20-40 amino acid residues, more preferably about 21-33 amino acid residues, and more preferably about 23-30 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, an LGR6 protein contains a signal sequence of about amino acids 1-23 of SEQ ID NO:2. The “signal sequence” is cleaved during processing of the mature protein. The mature LGR6 protein corresponds to amino acids 24 to 967 of SEQ ID NO:2. In another embodiment, an LGR6 protein caontains a signal sequence of about amino acids 1-25 of SEQ ID NO:11. The mature LGR6 protein corresponds to amino acids 26 to 968 of SEQ ID NO:11.

Accordingly in one embodiment of the invention, an LGR6 includes at least one, preferably 6 or 7, transmembrane domains and and/or at least one cytoplasmic loop, and/or at least one extracellular loop. In another embodiment, the LGR6 further includes an N-terminal extracellular domain and/or a C-terminal cytoplasmic domain. In another embodiment, the LGR6 can include six transmembrane domains, three cytoplasmic loops, and two extracellular loops, or can include six transmembrane domains, three extracellular loops, and two cytoplasmic loops. The former embodiment can further include an N-terminal extracellular domain. The latter embodiment can further include a C-terminal cytoplasmic domain. In another embodiment, the LGR6 can include seven transmembrane domains, three cytoplasmic loops, and three extracellular loops and can further include an N-terminal extracellular domain or a C-terminal cytoplasmic domain.

The LGR6 molecules of the present invention can further include at least one protein phosphorylation site, for example, at least one, two, three, four, five, six and preferably, seven Protein Kinase C sites; at least one, two, three, four, and preferably, five Casein Kinase II sites; and at least one, and preferably, two tyrosine kinase phosphorylation site. The LGR6 can additionally include at least one, five, ten, fifteen, sixteen, seventeen, eighteen, nineteen, twenty, and preferably twenty-one N-myristoylation sites; at least one N-glycosylation site; at least one glycosaminoglycan attachment site; and optionally, a signal sequence. For example, LGR6 contains predicted Protein Kinase C sites at about amino acids 19-21, 115-117, 142-144, 163-165, 420-422, 685-687 and 844-846 of SEQ ID NO:2, at about amino acids 52-54, 172-174 and 350-352 of SEQ ID NO:5, at about amino acids 276-278 and 454-456 of SEQ ID NO:8 and at about amino acids 19-21, 115-117, 142-144, 163-165, 507-509 and 685-687 of SEQ ID NO:11; predicted Casein Kinase II sites are located at about amino acids 328-331, 707-710, 862-865, 874-877 and 910-913 of SEQ ID NO:2, at about amino acids 372-375, 527-530 and 539-542 of SEQ ID NO:5 at about amino acids 97-100, 476-479, 631-634 and 643-646 of SEQ ID NO:8 and at about 328-331, 707-710, 862 to 865, 874-877 of SEQ ID NO:11; one, and preferably, two tyrosine kinase phosphopyration sites from about amino acids 469-475 of SEQ ID NO:2, at about amino acids 134-140 and 182-188 of SEQ ID NO:5, and at about amino acids 238-244 and 286-292 of SEQ ID NO:8 and at about amino acids 469-475 and 517-523 of SEQ ID NO:11; N-myristoylation sites from about amino acids 45-50, 99-104, 107-112, 380-385, 398-403, 483-488, 493-498, 513-518, 533-538, 563-568, 602-607, 612-617, 641-646, 652-657, 684-689, 698-703, 886-891, 922-927, 942-947, 949-954 and 960-965 of SEQ ID NO:2, from about amino acids 17-22, 148-153, 158-163, 228-233, 267-272, 277-282, 306-311, 317-322, 349-354, 363-368, 390-395, 587-592, 607-612, 613-618 and 625-630 of SEQ ID NO:5, and from about amino acids 149-154, 252-257, 262-267, 332-337, 371-376, 381-386, 410-415, 421-426, 453-458, 467-472, 494-499, 691-696, 711-716, 717-722 and 729-734 of SEQ ID NO:8 and from about amino acids 45-50, 99-104, 107-112, 127-132, 380-385, 483-488, 493-498, 563-568, 602-607, 612-617, 641-646, 652-657, 684-689, 698-703, 725-730, 922-927942-947, 948-953 and 960-965 of SEQ ID NO:11; two N-glycosylation sites from about amino acids 77-80 and 208-211 of SEQ ID NO:2, and from amino acids 1-4 and 48-51 of SEQ ID NO:5 and from about amino acids 77-80 and 208-211 of SEQ ID NO:11; and one glycosaminoglycan attachment site from about amino acids 638-641 of SEQ ID NO:2, from about amino acids 616-619 of SEQ ID NO:5, from about amino acids 720-723 of SEQ ID NO:8 and from about amino acids 951-954 of SEQ ID NO:11.

As the LGR6 proteins of the present invention may modulate LGR6-mediated activities, they may be useful for developing novel diagnostic and therapeutic agents for LGR6 associated disorders.

As used herein, a “LGR6-mediated activity” includes an activity which involves an LGR6 family member, associated with the regulation, sensing and/or transmission of an extracellular signal into a cell, for example, a neural cell, an endocrine cell or an adipose cell. LGR6-mediated activities include, for example, the interaction with (e.g., binding to) an extracellular signal (e.g., a glycohormone) or a cell surface receptor (e.g., an integrin receptor); the mobilization of an intracellular molecule that participates in a signal transduction pathway (e.g., adenylate cyclase or phosphatidylinositol 4,5-bisphosphate (PIP₂), inositol 1,4,5-triphosphate (IP₃)); the modulation of cell attachment; the modulation of neural development and maintenance; the modulation of thermogenesis in adipocytes, e.g., brown adipocytes, or muscle; the modulation of endocrine function; and/or the modulation of cardiovascular activities.

As used herein, an “LGR6 associated disorder” includes a disorder, disease or condition which is characterized by a misregulation of an LGR6-mediated activity. LGR6 associated disorders can detrimentally affect the regulation, sensing and/or transmission of an extracellular signal into a cell. As the LGR6 mRNA is expressed in adipose cells, e.g., brown fat, heart, brain and skeletal muscle, it is likely that LGR6 molecules of the present invention may be involved in disorders involving the activity of these cells. Examples of LGR6 associated disorders include a weight disorder, a metabolic disorder, a neural disorder (e.g., a central nervous system (CNS) disorder) an endocrine disorder, or a cardiovascular disorder.

For example, as the LGR6 mRNA is expressed in adipose cells, e.g., brown fat. Therefore, aberrant or abnormal LGR6 protein activity and/or nucleic acid expression may interfere with the normal weight control and metabolic functions. Disorders associated with body weight include disorders associated with abnormal body weight or abnormal control of body weight. Non-limiting examples of such disorders or diseases include, body weight disorders (e.g., anorexia, obesity and/or hyperphagia); eating disorders (e.g., anorexia nervosa and/or bulimia nervosa); cachexia; AIDS-related wasting; and cancer-related wasting.

In addition, LGR6 mRNA is expressed in the hypothalamus. Accordingly, in one embodiment, modulation of LGR6 activity has particular applicability in treating, hypothalamic dysfunction and/or disorders. As used herein, the term “hypothalamic dysfunction” includes a mis-regulated or aberrantly regulated function or activity attributed to the hypothalamus in an animal (e.g., in a human), for example, a mis-regulated or aberrantly regulated hypothalamic activity, as described herein. As used herein the term “hypothalamic disorder” includes a disease or disorder characterized by at least one phenotypic manifestation (e.g., a clinically detectable manifestation or symptom) of a hypothalamic dysfunction, as defined herein. The term “hypothalamic activity”, as used herein, includes at least one or more of the following activities: (1) modulation (e.g. repression or stimulation) of brain anabolic circuits or pathways; (2) modulation e.g., repression or stimulation) of brain catabolic pathways; (3) modulation of food intake and/or feeding behavior (e.g., stimulation of or inhibition/suppression of food intake and/or feeding behavior); (4) modulation of energy expenditure (e.g., suppression or stimulation of energy expenditure); (5) regulation of energy homeostasis; (6) regulation of body fat mass; (7) regulation of body temperature; (8) regulation of the sleep-wake cycle; (9) regulation of memory and/or behavior; (10) control of thirst; and (11) regulation of autonomic nervous system function; (12) modulation of cellular signal transduction, either in vitro or in vivo; (13) regulation of gene transcription in a cell expressing an LGR6 protein; (14) regulation of cellular proliferation; (15) regulation of cellular differentiation; (16) regulation of development; (17) regulation of cell death; (18) regulation of inflammation; and (19) regulation of respiratory cell function. Modulation of an LGR6 activity as described above may be included as part of a multi-drug regime that targets multiple sites within the weight regulatory system, temperature regulatory system, sleep-wake cycle control system, memory and/or behavior regulatory systems, thirst regulatory system and/or autonomic nervous system.

CNS disorders such as cognitive and neurodegenerative disorders, examples of which include, but are not limited to, Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, movement disorders, progressive supranuclear palsy, epilepsy, AIDS related dementia, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Further CNS-related disorders include, for example, those listed in the American Psychiatric Association's Diagnostic and Statistical manual of Mental Disorders (DSM), the most current version of which is incorporated herein by reference in its entirety.

As used herein, the term “cardiovascular disorder” includes a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. Cardiovascular system disorders in which the LGR6 molecules of the invention may be directly or indirectly involved include arteriosclerosis, atherosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, valvular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, cardiomyopathies (e.g., dilated cardiomyopathy, idiopathic cardiomyopathy), myocardial infarction, coronary artery disease, coronary artery spasm, and arrhythmias.

As used herein, the term “congestive heart failure” includes a condition characterized by a diminished capacity of the heart to supply the oxygen demands of the body. Symptoms and signs of congestive heart failure include diminished blood flow to the various tissues of the body, accumulation of excess blood in the various organs, e.g., when the heart is unable to pump out the blood returned to it by the great veins, exertional dyspnea, fatigue, and/or peripheral edema, e.g., peripheral edema resulting from left ventricular dysfunction. Congestive heart failure may be acute or chronic. The manifestation of congestive heart failure usually occurs secondary to a variety of cardiac or systemic disorders that share a temporal or permanent loss of cardiac function. Examples of such disorders include hypertension, coronary artery disease, valvular disease, and cardiomyopathies, e.g., hypertrophic, dilative, or restrictive cardiomyopathies. Congestive heart failure is described in, for example, Cohn J. N. et al. (1998) American Family Physician 57:1901-04, the contents of which are incorporated herein by reference.

As used herein, an “endocrine disorder” refers to an abnormal hormonally-mediated metabolic function of the body such as controlling the rates of chemical reactions in the cells, the transport of substances through cell membranes or other aspects of cellular metabolism such as growth and secretion. Non-limiting examples of endocrine disorders include hypothyroidism, hyperthyroidism, dwarfism, giantism, acromegaly, among others (Guyton, A. C. Medical Physiology 6^{th} Ed. W.B. Saunders Co. Philadelphia).

The LGR6 protein may participate in signaling pathways within cells, e.g., signaling pathways involved in proliferation or differentiation. As used herein, a signaling pathway refers to the modulation (e.g., the stimulation or inhibition) of a cellular function/activity upon the binding of a ligand to the GPCR (LGR6 protein). In some embodiments, the LGR6 proteins of the invention may share the same ligands as LGR4 and LGR5 proteins. Examples of such functions include mobilization of intracellular molecules that participate in a signal transduction pathway, e.g., adenylate cyclase, or phosphatidylinositol 4,5-bisphosphate (PIP₂), inositol 1,4,5-triphosphate (IP₃); production or secretion of molecules; alteration in the structure of a cellular component; cell proliferation, e.g., synthesis of DNA; cell migration; cell attachment; cell differentiation; and cell survival. Since the LGR6 protein is expressed substantially in adipose tissues (e.g., brown fat), brain, heart, skeletal muscle, examples of cells participating in an LGR6 signaling pathway include adipose cells, brain cells, heart and skeletal muscle cells.

Depending on the type of cell, the response mediated by the LGR6 protein/ligand binding may be different. For example, in some cells, binding of a ligand to an LGR6 protein may stimulate an activity such as adhesion, migration, differentiation, and the like through cyclic AMP metabolism or phosphatidylinositol turnover. Regardless of the cellular activity modulated by LGR6, it is universal that as a GPCR, the LGR6 protein interacts with a “G protein” to produce one or more secondary signals in a variety of intracellular signal transduction pathways, e.g., through cyclic AMP metabolism or phosphatidylinositol turnover, in a cell.

The term “G proteins” refers to a family of heterotrimeric proteins composed of α, β, and γ subunits, which bind guanine nucleotides. These proteins are usually linked to cell surface receptors, e.g., receptors containing seven transmembrane domains, such as the ligand receptors. Following ligand binding to the receptor, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits. Examples of mammalian G proteins include Gi, Go, Gq, Gs and Gt. G proteins are described extensively in Lodish H. et al. Molecular Cell Biology, (Scientific American Books Inc., New York, N.Y., 1995), the contents of which are incorporated herein by reference.

Another signaling pathway in which the LGR6 protein may participate is the cAMP turnover pathway. As used herein, “cyclic AMP turnover and metabolism” includes molecules involved in the turnover and metabolism of cyclic AMP (cAMP), as well as to the activities of these molecules. Cyclic AMP is a second messenger produced in response to ligand induced stimulation of certain G protein coupled receptors. In the ligand signaling pathway, binding of ligand to a ligand receptor can lead to the activation of the enzyme adenylate cyclase, which catalyzes the synthesis of cAMP. The newly synthesized cAMP can in turn activate a cAMP-dependent protein kinase. cAMP pathways have been implicated in the regulation of thermogenesis and lipolysis in brown fat.

As used herein, the phrase “phosphatidylinositol turnover and metabolism” includes the molecules involved in the turnover and metabolism of phosphatidylinositol 4,5-bisphosphate (PIP₂) as well as to the activities of these molecules. PIP₂ is a phospholipid found in the cytosolic leaflet of the plasma membrane. Binding of a ligand to the LGR6 activates, in some cells, the plasma-membrane enzyme phospholipase C that in turn can hydrolyze PIP₂ to produce 1,2-diacylglycerol (DAG) and inositol 1,4,5-triphosphate (IP₃). Once formed IP₃ can diffuse to the endoplasmic reticulum surface where it can bind an IP₃ receptor. IP₃ binding can induce opening of the channel, allowing calcium ions to be released into the cytoplasm. IP₃ can also be phosphorylated by a specific kinase to form inositol 1,3,4,5-tetraphosphate (IP₄), a molecule which can cause calcium entry into the cytoplasm from the extracellular medium. IP₃ and IP₄ can subsequently be hydrolyzed very rapidly to the inactive products inositol 1,4-biphosphate (IP₂) and inositol 1,3,4-triphosphate, respectively. These inactive products can be recycled by the cell to synthesize PIP₂. The other second messenger produced by the hydrolysis of PIP₂, namely 1,2-diacylglycerol (DAG), remains in the cell membrane where it can serve to activate the enzyme protein kinase C. Protein kinase C is usually found soluble in the cytoplasm of the cell, but upon an increase in the intracellular calcium concentration, this enzyme can move to the plasma membrane where it can be activated by DAG. The activation of protein kinase C in different cells results in various cellular responses such as the phosphorylation of glycogen synthase, or the phosphorylation of various transcription factors, e.g., NF-kB. The language “phosphatidylinositol activity”, as used herein, includes an activity of PIP₂ or one of its metabolites.

In one embodiment, isolated proteins of the present invention, preferably LGR6 proteins, have an amino acid sequence sufficiently homologous to the amino acid sequence of SEQ ID NO:2, or are encoded by a nucleotide sequence sufficiently homologous to SEQ ID NO:1, or SEQ ID NO:3. In another embodiment, isolated proteins of the present invention, preferably LGR6 proteins, have an amino acid sequence sufficiently homologous to the amino acid sequence of SEQ ID NO:5 or SEQ ID NO:8, or are encoded by a nucleotide sequence sufficiently homologous to SEQ ID NO:4, SEQ ID NO:6, SEQ ID NO:7 or SEQ ID NO:9. In yet another embodiment, isolated proteins of the present invention, preferably LGR6 proteins, have an amino acid sequence sufficiently homologous to the amino acid sequence of SEQ ID NO:11, or are encoded by a nucleotide sequence sufficiently homologous to SEQ ID NO:10 or SEQ ID NO:12. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 60% homology, preferably 65% homology, more preferably 70%-80%, and even more preferably 90-95% homology across the amino acid sequences of the domains and contain at least one and preferably two structural domains or motifs, are defined herein as sufficiently homologous. Furthermore, amino acid or nucleotide sequences which share at least 60%, preferably 65%, more preferably 70-80%, or 90-95% homology and share a common functional activity are defined herein as sufficiently homologous.

As used interchangeably herein, a “LGR6 activity”, “biological activity of LGR6” or “functional activity of LGR6”, refers to an activity exerted by an LGR6 protein, polypeptide or nucleic acid molecule on an LGR6 responsive cell or on an LGR6 protein substrate, as determined in vivo, or in vitro, according to standard techniques. In one embodiment, an LGR6 activity is a direct activity, such as an association with an LGR6-target molecule. As used herein, a “target molecule” or “binding partner” is a molecule with which an LGR6 protein binds or interacts in nature, such that LGR6-mediated function is achieved. An LGR6 target molecule can be a non-LGR6 molecule or an LGR6 protein or polypeptide of the present invention. In an exemplary embodiment, an LGR6 target molecule is a ligand or a G protein. Alternatively, an LGR6 activity is an indirect activity, such as a cellular signaling activity mediated by interaction of the LGR6 protein with a ligand or a G-protein. The biological activities of LGR6 are described herein. For example, the LGR6 proteins of the present invention can have one or more of the following activities: (1) interact with (e.g., bind to) an extracellular signal, e.g., a glycohormone, or a cell surface receptor; (2) mobilize an intracellular molecule that participates in a signal transduction pathway such as adenylate cyclase or phosphatidylinositol 4,5-bisphosphate (PIP₂), inositol 1,4,5-triphosphate (IP₃); (3) modulate cell attachment; (4) modulate neural development and maintenance; (5) modulate thermogenesis in adipocytes, e.g., brown adipocytes, or muscle; (6) modulate endocrine function; and (7) modulate cardiovascular activities.

Accordingly, another embodiment of the invention features isolated LGR6 proteins and polypeptides having an LGR6 activity. Preferred proteins are LGR6 proteins having at least one extracellular domain, at least one leucine-rich repeat, at least one RGD-cell attachment site, at least one transmembrane domain and at least one cytoplasmic domain, and preferably, an LGR6 activity. Other preferred proteins are LGR6 proteins having at least one extracellular domain and, preferably, an LGR6 activity. Other preferred proteins are LGR6 proteins having at least one leucine-rich repeat and, preferably, an LGR6 activity. Other preferred proteins are LGR6 proteins having at least one RGD-cell attachment site and, preferably, an LGR6 activity. Other preferred proteins are LGR6 proteins having at least one transmembrane domain and, preferably, an LGR6 activity. Other preferred proteins are LGR6 proteins having at least one cytoplasmic domain, and, preferably, an LGR6 activity. Other preferred proteins are LGR6 proteins having at least one extracellular domain, at least one leucine-rich repeat, at least one RGD-cell attachment site, at least one transmembrane domain and at least one cytoplasmic domain, and are, preferably, encoded by a nucleic acid molecule having a nucleotide sequence which hybridizes under stringent hybridization conditions to a nucleic acid molecule comprising the nucleotide sequence of SEQ ID NO:1, SEQ ID NO:3, SEQ ID NO:4, SEQ ID NO:6, SEQ ID NO:7, SEQ ID NO:9, SEQ ID NO:10 or SEQ ID NO:12.

The nucleotide sequence of the isolated mouse LGR6 cDNA (clone ftmzb048h10) and its predicted amino acid sequence are shown in FIG. 1 and in SEQ ID NOs:1 and 2, respectively. A plasmid containing the nucleotide sequence encoding human LGR6 was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The mouse LGR6 cDNA (clone ftmzb048h10) sequence (SEQ ID NO:1), which is approximately 3637 nucleotides long including untranslated regions, contains a predicted methionine-initiated coding sequence of about 2900 nucleotides (nucleotides 222-3122 of SEQ ID NO:1; SEQ ID NO:3) which encodes a 967 amino acid protein (SEQ ID NO:2). The mouse LGR6 protein of SEQ ID NO:2 includes an amino-terminal hydrophobic amino acid sequence, consistent with a signal sequence, of about 23 amino acids (from amino acid I to about amino acid 23 of SEQ ID NO:2), which upon protease removal results in the production of the mature protein.

The mature protein is approximately 944 amino acid residues in length (from about amino acid 24 to amino acid 967 of SEQ ID NO:2). Mouse LGR6 contains one long extracellular domain located at about amino acid residues 1-563 of SEQ ID NO:2; sixteen leucine-rich repeats (PF00560) are located at about amino acid residues 67 to 90, 91 to 114, 115 to 138, 139 to 162, 163 to 186, 187 to 210, 211 to 234, 235 to 257, 258 to 281, 282 to 305, 306 to 329, 330 to 352, 353 to 375, 376 to 398, 399 to 422, and 423 to 446 of SEQ ID NO:2 of SEQ ID NO:2; one RGD cell attachment site is located at about amino acid residues 760-762 of SEQ ID NO:2; seven transmembrane domains which extend from about amino acid 564 (extracellular end) to about amino acid 590 (cytoplasmic end) of SEQ ID NO:2; from about amino acid 598 (cytoplasmic end) to about amino acid 620 (extracellular end) of SEQ ID NO:2; from about amino acid 645 (extracellular end) to about amino acid 669. (cytoplasmic end) of SEQ ID NO:2; from about amino acid 684 (cytoplasmic end) to about amino acid 704 (extracellular end); from about amino acid 731 (extracellular end) to about amino acid 751 (cytoplasmic end); from about amino acid 773 (cytoplasmic end) to about amino acid 798 (extracellular end); and from about amino acid 812 (extracellular end) to about amino acid 834 (cytoplasmic end); three cytoplasmic loops found at about amino acids 591-597, 670-683, and 752-772 of SEQ ID NO:2; three extracellular loops found at about amino acid 621-644, 705-730 and 799-811 of SEQ ID NO:2; and a C-terminal cytoplasmic domain is found at about amino acid residues 835-968 of SEQ ID NO:2.).

The mouse LGR6 protein (clone ftmzb048h10 protein) additionally contains seven predicted protein kinase C phosphorylation sites (PS00005) from amino acids 19-21, 115-117, 142-144, 163-165, 420-422, 685-687 and 844-846 of SEQ ID NO:2; five casein kinase II phosphorylation sites (PS00006) from amino acids acids 328-331, 707-710, 862-865, 874-877 and 910-913 of SEQ ID NO:2; one tyrosine kinase phosphorylation site (PS00007) from amino acid 469-475 of SEQ ID NO:2; twenty-one N-myristoylation sites (PS00008) from amino acids 45-50, 99-104, 107-112, 380-385, 398-403, 483-488, 493-498, 513-518, 533-538, 563-568, 602-607, 612-617, 641-646, 652-657, 684-689, 698-703, 886-891, 922-927, 942-947, 949-954 and 960-965 of SEQ ID NO:2; two N-glycosylation sites from about amino acids 77-80 and 208-211 of SEQ ID NO:2; and one glycosaminoglycan attachment site from about amino acids 638-641 of SEQ ID NO:2.

The nucleotide sequence of the isolated full length human LGR6 cDNA (clone Fbh150881) and its predicted amino acid sequence are shown in FIGS. 14 and 15, and in SEQ ID NOs:10 and 11, respectively. A plasmid containing the nucleotide sequence encoding human LGR6 was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the are and is not an admission that a deposit is required under 35 U.S.C. §112.

The human LGR6 cDNA (clone 15088) sequence (SEQ ID NO:10), which is approximately 3492 nucleotides long including untranslated regions, contains a predicted methionine-initiated coding sequence of about 2901 nucleotides (nucleotides 104-3004 od SEQ ID NO:10, SEQ ID NO:12) which encodes a 968 amino acid protein (SEQ ID NO:11). The human LGR6 protein of SEQ ID NO:11 includes an amino-terminal hydrophobic amino acid sequence, consistent with a signal sequence, of about amino acids (from amino acid 1 to about amino acid 25 of SEQ ID NO:11), which upon protease removal results in the production of the mature protein.

The mature protein is approximately 943 amino acid residues in length (from about amino acid 25 to amino acid 968 of SEQ ID NO:11). Human LGR6 is localized in the endoplasmic reticulum, the mitochondria, the vesicles of the secretory system and the Golgi. Human LGR6 contains sixteen leucine-rich repeats (PF00560) are located at about amino acid residues 67 to 90, 91 to 114, 1150 to 138, 139 to 162, 163 to 186, 187 to 210, 211 to 234, 235 to 257, 258 to 281, 282 to 305, 306 to 329, 330 to 352, 353 to 375, 376 to 398, 399 to 422, and 423 to 446 of SEQ ID NO:11; one RGD cell attachment site is located at about amino acid residues 760-762 of SEQ ID NO:11; six transmembrane domains which extend from about amino acid 566 (extracellular end) to about amino acid 590 (cytoplasmic end) of SEQ ID NO:11; from about amino acid 599 (cytoplasmic end) to about amino acid 621 (extracellular end) of SEQ ID NO:11; from about amino acid 646 (extracellular end) to about amino acid 665 (cytoplasmic end) of SEQ ID NO:11; from about amino acid 688 (cytoplasmic end) to about amino acid 709 (extracellular end) of SEQ ID NO:11; from about amino acid 728 (extracellular end) to about amino acid 752 (cytoplasmic end) of SEQ ID NO:11; and from about amino acid 777 (cytoplasmic end) to about amino acid 801 (extracellular end) of SEQ ID NO:11.

The human LGR6 protein (clone 15088) additionally contains six predicted protein kinase C phosphorylation sites (PS00005) from amino acids 19-21, 115-117, 142-144, 163-165, 507-509 and 685-687 of SEQ ID NO:11; four casein kinase II phosphorylation sites (PS00006) from amino acids acids 328-331, 707-710, 862-865 and 874-877 of SEQ ID NO:11; two tyrosine kinase phosphorylation sites (PS00007) from amino acid 469-475 and 517-523 of SEQ ID NO:11; nineteen N-myristoylation sites (PS00008) from amino acids amino acids 45-50, 99-104, 107-112, 127-132, 380-385, 483-488, 493-498, 563-568, 602-607, 612-617, 641-646, 652-657, 684-689, 698-703, 725-730, 922-927942-947, 948-953 and 960-965 of SEQ ID NO: 11; two N-glycosylation sites from about amino acids 77-80 and 208-211 of SEQ ID NO:11; and one glycosaminoglycan attachment site from about amino acids 951-954 of SEQ ID NO: 11; three prokaryotic membrane lipoprotein lipid attachment sitees from about amino acids 605-615, 663-673 and 894-904; one leucine zipper pattern from about amino acid 57-78; one C-terminal targeting signal from about amino acid 965-968; one Glycoprotein EGF-like Domain receptor from about amino acids 70-433.

The nucleotide sequence of the isolated human LGR6 cDNA (clone fahr) and its predicted amino acid sequence are shown in FIGS. 4 and 5, and in SEQ ID NOs:4 and 5, respectively. A plasmid containing the nucleotide sequence encoding human fahr was deposited with American Type Culture Collection (ATCC), 10801 University Boulevard, Manassas, Va. 20110-2209, on ______ and assigned Accession Number ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. § 112.

In one embodiment the human LGR6 cDNA (clone fahr) sequence (SEQ ID NO:1), which is approximately 2486 nucleotides long including untranslated regions, contains coding sequence of about 1899 nucleotides (nucleotides 1-1899 of SEQ ID NO:4; SEQ ID NO:6) which encodes a 633 amino acid protein (SEQ ID NO:5). An alignment of clone fahr and clone ftmzb048h10 is shown in FIG. 7.

The protein encoded by human LGR6 cDNA (clone fahr) is approximately 633 amino acid residues in length (SEQ ID NO:5) and contains two leucine-rich repeat located at about amino acid residues 64 to 87 and 88 to 111 of SEQ ID NO:5; one RGD cell attachment site is located at about amino acid residues 425-467 of SEQ ID NO:5; seven transmembrane domains which extend from about amino acid 230 (extracellular end) to about amino acid 256 (cytoplasmic end) of SEQ ID NO:5; from about amino acid 264 (cytoplasmic end) to about amino acid 286 (extracellular end) of SEQ ID NO:5; from about amino acid 311 (extracellular end) to about amino acid 336 (cytoplasmic end) of SEQ ID NO:5; from about amino acid 350 (cytoplasmic end) to about amino acid 370 (extracellular end) of SEQ ID NO:5; from about amino acid 397 (extracellular end) to about amino acid 417 (cytoplasmic end) of SEQ ID NO:5; from about amino acid 440 (cytoplasmic end) to about amino acid 464 (extracellular end) of SEQ ID NO:5; and from about amino acid 478 (extracellular end) to about amino acid 500 (cytoplasmic end); three cytoplasmic loops found at about amino acids 257-263, 337-349 and 418-439 of SEQ ID NO:5; three extracellular loops found at about amino acid 287-310, 371-396 and 465-477 of SEQ ID NO:5; and a C-terminal cytoplasmic domain is found at about amino acid residues 501-633 of SEQ ID NO:5.

The human LGR6 protein additionally contains three predicted protein kinase C phosphorylation sites (PS00005) from amino acids 52-54, 172-174 and 350-352 of SEQ ID NO:5; three casein kinase II phosphorylation sites (PS00006) from amino acids acids 372-375, 527-530 and 539-542 of SEQ ID NO:5; two tyrosine kinase phosphorylation site (PS00007) from amino acid 134-140 and 182-188 of SEQ ID NO:5; fifteen N-myristoylation sites (PS00008) from amino acids 17-22, 148-153, 158-163, 228-233, 267-272, 277-282, 306-311, 317-322, 349-354, 363-368, 390-395, 587-592, 607-612, 613-618 and 625-630 of SEQ ID NO:5; two N-glycosylation sites from about amino acids 1-4 and 48-51 of SEQ ID NO:5; and one glycosaminoglycan attachment site from about amino acids 616-619 of SEQ ID NO:5.

In another embodiment the human LGR6 cDNA (clone fahr) sequence (SEQ ID NO:7), which is approximately 2711 nucleotides long including untranslated regions, contains coding sequence of about 2208 nucleotides (nucleotides 1-2208 of SEQ ID NO:7; SEQ ID NO:9) which encodes a 736 amino acid protein (SEQ ID NO:5). An alignment of the nucleotide sequences and amino acid sequences of clone fahr and clone ftmzb048h10 is shown in FIGS. 12 and 13, respectively.

The protein encoded by human LGR6 cDNA (SEQ ID NO:7) is approximately 736 amino acid residues in length (SEQ ID NO:8) and contains leucine-rich repeat domains located at about amino acid residues 4-26, 27-50, 51-74, 75-97, 98-121, 122-143, 144-167, 168-191, and 192-215 of SEQ ID NO:8; one RGD cell attachment site is located at about amino acid residues 529-531 of SEQ ID NO:8; seven transmembrane domains which extend from about amino acid 333 (extracellular end) to about amino acid 359 (cytoplasmic end) of SEQ ID NO:8; from about amino acid 367 (cytoplasmic end) to about amino acid 389 (extracellular end) of SEQ ID NO:8; from about amino acid 414 (extracellular end) to about amino acid 439 (cytoplasmic end) of SEQ ID NO:8; from about amino acid 453. (cytoplasmic end) to about amino acid 473 (extracellular end) of SEQ ID NO:8; from about amino acid 500 (extracellular end) to about amino acid 520 (cytoplasmic end) of SEQ ID NO:8; from about amino acid 543 (cytoplasmic end) to about amino acid 567 (extracellular end) of SEQ ID NO:8; and from about amino acid 581 (extracelluar end) to about amino acid 603 (cytoplasmic end) of SEQ ID NO:8; two 7 tm_{—}1 domains at about amino acid residues 404-431 and 553-596 of SEQ ID NO:8; three cytoplasmic loops found at about amino acids 360-366, 440-452 and 521-542 of SEQ ID NO:8; three extracellular loops found at about amino acid residues 390-413, 474-499 and 568-580 of SEQ ID NO:8; and a C-terminal cytoplasmic domain is found at about amino-acid residues 604-736 of SEQ ID NO:8.

The human LGR6 protein additionally contains two predicted protein kinase C phosphorylation sites (PS00005) from amino acids 276-278 and 454-456 of SEQ ID NO:8; four casein kinase II phosphorylation sites (PS00006) from amino acids acids 97-100, 476-479, 631-634 and 643-646 of SEQ ID NO:8; two tyrosine kinase phosphorylation site (PS00007) from amino acics 238-244 and 286-292 of SEQ ID NO:8; fifteen N-myristoylation sites (PS00008) from amino acids acids 149-154, 252-257, 262-267, 332-337, 371-376, 381-386, 410-415, 421-426, 453-458, 467-472, 494-499, 691-696, 711-716, 717-722 and 729-734 of SEQ ID NO:8; and one glycosaminoglycan attachment site from about amino acids 720-723 of SEQ ID NO:8.

For general information regarding PFAM identifiers, PS prefix and PF prefix domain identification numbers, refer to Sonnhammer et al. (1997) Protein 28:405-420 and http://www.psc.edu/general/software/packages/pfam/pfam.html.

As detected using a partial sequence of the mouse clone ftmzb048h10 gene (clone jambb01d11), this gene is expressed in mouse brown fat (with undetectable levels of expression in white fat), with lower levels of expression detected in the mouse heart and the brain. In the developing mouse (embryonic day 17), the clone ftmzb048h10 gene is expressed in brown fat, smooth muscle of the heart vessel, smooth muscle of the bronchiole, epithelial cell layer of the trachea, mesenchymal cell layer of the tooth, intravertebral disk and developing flat bone of the skull. In the adult mouse brain, this gene is expressed in the hypothalamus (arcuate nucleus and periventricular nucleus), eppendymal cell layer of the third ventricle close to the arcuate nucleus region, the supraoptic nucleus, the cortex, hippocampus, paraventral, paracentral, medio-dorsal and intradorsal thalamic nuclei.

In humans, the distribution of the LGR6 gene was found in decreasing order of abundance in the human heart, brain and skeletal muscle.

The LGR6 nucleic acids and polypeptides of the invention may play roles in normal and pathological processes involving the cells and tissues that express them, or cells and tissues that contact said LGR6 polypeptides. For example, since LGR6 molecules are expressed in the heart, as shown in Example 2, LGR6 molecules may be involved in cardiovascular disorders including, but not limited to, atherosclerosis, ischaemia reperfusion injury, cardiac hypertrophy, hypertension, coronary artery disease, myocardial infarction, arrythmia, cardiomyopathies, and congestive heart failure. Similarly, since the LGR6 molecules are expressed in adipose tissues, e.g., brown fat cells, these molecules may be involved in, for example, thermogenesis. Accordingly, the LGR6 molecules may be involved in weight disorders, including, e.g. obesity, cachexia and anorexia. Similarly, the expression of LGR6 molecules in the human skeletal muscle suggests that these molecules may be involved in thermogenesis in humans.

Various aspects of the invention are described in further detail in the following subsections:

I. Isolated Nucleic Acid Molecules

One aspect of the invention pertains to isolated nucleic acid molecules that encode LGR6 proteins or biologically active portions thereof, as well as nucleic acid fragments sufficient for use as hybridization probes to identify LGR6-encoding nucleic acid molecules (e.g., LGR6 mRNA) and fragments for use as PCR primers for the amplification or mutation of LGR6 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated LGR6 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO: 1, SEQ ID NO:3, SEQ ID NO: 4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______, or ______ a portion thereof, can be isolated using standard molecular biology techniques and the sequence information provided herein. Using all or portion of the nucleic acid sequence of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______ as a hybridization probe, LGR6 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______ or ______ can be isolated by the polymerase chain reaction (PCR) using synthetic oligonucleotide primers designed based upon the sequence of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to LGR6 nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1. The sequence of SEQ ID NO:1 corresponds to mouse LGR6 cDNA (clone ftmzb048h10 cDNA). This cDNA comprises sequences encoding the mouse LGR6 protein (i.e., “the coding region”, from nucleotides 222-3122), as well as 5′ untranslated sequences (nucleotides 1-221) and 3′ untranslated sequences (nucleotides 3123-3637) of SEQ ID NO:1. Alternatively, the nucleic acid molecule can comprise only the coding region of SEQ ID NO:1 (e.g., nucleotides 222-3122, corresponding to SEQ ID NO:3).

In another preferred embodiment, an isolated nucleic acid molecule of the invention comprises the nucleotide sequence shown in SEQ ID NO:4. The sequence of SEQ ID NO:4 corresponds to the human LGR6-cDNA (clone fahr cDNA). This cDNA comprises sequences encoding the human LGR6 protein (i.e., “the coding region”, from nucleotides 1-1899), as well as 3′ untranslated sequences (nucleotides 1900-2486) of SEQ ID NO:4. Alternatively, the nucleic acid molecule can comprise only the coding region of SEQ ID NO:4 (e.g., nucleotides 1-1899, corresponding to SEQ ID NO:6).

In yet another preferred embodiment, an isolated nucleic acid molecule of the invention comprises the nucleotide sequence shown in SEQ ID NO:7. The sequence of SEQ ID NO:7 corresponds to the human LGR6 cDNA (clone fahr cDNA). This cDNA comprises sequences encoding the human LGR6 protein (i.e., “the coding region”, from nucleotides 1-2208), as well as 3′ untranslated sequences (nucleotides 2209-2711) of SEQ ID NO:7. Alternatively, the nucleic acid molecule can comprise only the coding region of SEQ ID NO:7 (e.g., nucleotides 1-2208, corresponding to SEQ ID NO:9).

In yet another preferred embodiment, an isolated nucleic acid molecule of the invention comprises the nucleotide sequence shown in SEQ ID NO:10. The sequence of SEQ ID NO:10 corresponds to the full length nucleotide sequence of human LGR6 (clone Fbh150881). This sequence comprises sequences encoding the human LGR6 protein (i.e., “the coding region” from nucleotides 104 to 3004), as well as 3′ untranslated sequences (nucleotides 1-103), as well as 5′ untranslated sequences (nucleotides 3005-3492) of SEQ ID NO:10. Alternatively, the nucleic acid molecule can comprise only the coding region of SEQ ID NO:10 (e.g., nucleotides 104-3004, corresponding to SEQ ID NO:12).

In another preferred embodiment, an isolated nucleic acid molecule of the invention comprises a nucleic acid molecule which is a complement of the nucleotide sequence shown in SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of The DNA insert of the plasmid deposited with ATCC as Accession Number ______, ______ or ______, or a portion of any of these nucleotide sequences. A nucleic acid molecule which is complementary to the nucleotide sequence shown in SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, is one which is sufficiently complementary to the nucleotide sequence shown in SEQ ID NO:1, SEQ ID NO:3, SEQ ID NO:4, SEQ ID NO:6, SEQ ID NO:7, SEQ ID NO:9, SEQ ID NO:10, SEQ ID NO: 12, or the nucleotide sequence of the DNA insert of the plasmid deposited with ATCC as Accession Number ______, ______ or ______, such that it can hybridize to the nucleotide sequence shown in SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, thereby forming a stable duplex.

In still another preferred embodiment, an isolated nucleic acid molecule of the present invention comprises a nucleotide sequence which is at least about 60%, 65%, 70%, 75%, 80%, 85%, 90%, 95% or more homologous to the entire length of the nucleotide sequence shown in SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the entire length of the nucleotide sequence of the DNA insert of the plasmid deposited with ATCC as Accession Number ______, ______ or ______, or a portion of any of these nucleotide sequences.

A. LGR6 Nucleic Acid Fragments

Moreover, the nucleic acid molecule of the invention can comprise only a portion of the nucleic acid sequence of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, for example a fragment which can be used as a probe or primer or a fragment encoding a biologically active portion of an LGR6 protein, e.g., a fragment comprising nucleotides 422 to 563 of SEQ ID NO:1, which encodes a leucine-rich repeat of mouse LGR6. Alernatively, a fragment comprising nucleotides 192 to 362 of SEQ ID NO:4, which encodes a leucine-rich repeat of human LGR6 can be used. The nucleotide sequence determined from the cloning of the LGR6 gene allows for the generation of probes and primers designed for use in identifying and/or cloning other LGR6 family members, as well as LGR6 homologues from other species.

The probe/primer typically comprises substantially purified oligonucleotide. The oligonucleotide typically comprises a region of nucleotide sequence that hybridizes under stringent conditions to at least about 12 to 15, preferably about 20 to 25, more preferably about 30, 35, 40, 45, 50, 55, 60, 65, or 75 consecutive nucleotides of a sense sequence of SEQ ID NO:1, SEQ ID NO:3, SEQ ID NO:4, SEQ ID NO:6, SEQ ID. NO:7, SEQ ID NO:9, SEQ ID NO:10, SEQ ID NO:12, or the nucleotide sequence of the DNA insert of the plasmid deposited with ATCC as Accession Number ______, ______ or ______, of an anti-sense sequence of SEQ ID NO: 1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, or of a naturally occurring allelic variant or mutant of SEQ ID NO: 1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______.

In an exemplary embodiment, a nucleic acid molecule of the present invention comprises a nucleotide sequence which is 439, 440, 450-500, 500-550, 537, 550-600, 600-650, 650-700, 700-750, 750-800, 800-850, 850-900, 950-1000, 1100-1200, 1200-1500, 1500-2000, 2000-2500, 2500-3000, 3000-3500 and 3500-3600 nucleotides in length and hybridizes under stringent hybridization conditions to a nucleic acid molecule of SEQ ID NO:1, or 439, 440, 450-500, 500-550, 537, 550-600, 600-650, 650-700, 700-750, 750-800, 800-850, 850-900, 950-1000, 1100-1200, 1200-1500, 1500-2000, 2000-2500, 2500-3000, 3000-3500 and 3500-3600 nucleotides in length and hybridizes under stringent hybridization conditions to a nucleic acid molecule of SEQ ID NO:3, or the nucleotide sequence of the DNA insert of the plasmid deposited with ATCC as Accession Number ______.

In yet another exemplary embodiment, a nucleic acid molecule of the present invention comprises a nucleotide sequence which is 481, 490-500, 500-550, 537, 550-600, 600-650, 650-700, 700-750, 750-800, 800-850, 850-900, 950-1000, 1100-1200, 1200-1500, 1500-2000, or 2000-2300 nucleotides in length and hybridizes under stringent hybridization conditions to a nucleic acid molecule of SEQ ID NO:4, or 481, 490-500, 500-550, 537, 550-600, 600-650, 650-700, 700-750, 750-800, 800-850, 850-900, 950-1000, 1100-1200, 1200-1500, 1500-2000, or 2000-2300 nucleotides in length and hybridizes under stringent hybridization conditions to a nucleic acid molecule of SEQ ID NO:6, or the nucleotide sequence of the DNA insert of the plasmid deposited with ATCC as Accession Number ______.

In another embodiment, a nucleic acid molecule of the present invention comprises a nucleotide sequence which is 167, 170-200, 200-220, 220-240, 240-260, 260-280, 280-300, 300-320, 320-340, 340-360, 360-380, 380-400, 400-420, 420-440, 440-460, 460-480, 490-500, 500-550, 537, 550-600, 600-650, 650-700, 700-750, 750-800, 800-850, 850-900, 950-1000, 1100-1200, 1200-1500, or 1500-1899 nucleotides in length and hybridizes under stringent hybridization conditions to a nucleic acid molecule comprising nucleotides 1-1899 of SEQ ID NO:4, or SEQ ID NO:6, or the nucleotide sequence of the DNA insert of the plasmid deposited with ATCC as Accession Number ______.

In yet another embodiment, a nucleic acid molecule of the present invention comprises a nucleotide sequence which is 250-500, 500-750, 750-1000, 1000-1200, 1200-1400, 1400-1600, 1600-1800, 1800-2000, 2000-2174, 2175, 2176-2200, 2200-2400, 2400-2600, 2600 or more nucleotides in length and hybridizes under stringent hybridization conditions to a nucleic acid molecule comprising SEQ ID NO:7, or SEQ ID NO:9, or the nucleotide sequence of the DNA insert of the plasmid deposited with ATCC as Accession Number ______.

In yet another exemplary embodiment, a nucleic acid molecule of the present invention comprises a nucleotide sequence which is 1-50, 50-150, 150-250, 250-350, 350-438, 439, 440, 450-500, 500-550, 537, 550-600, 600-650, 650-700, 700-750, 750-800, 800-850, 850-900, 950-1000, 1100-1200, 1200-1500, 1500-2000, 2000-2500, 2500-3000, 3000-3500 and 3500-3600 nucleotides in length and hybridizes under stringent hybridization conditions to a nucleic acid molecule of SEQ ID NO:10, or is 1-50, 50-150, 150-250, 250-350, 350-438, 439, 440, 450-500, 500-550, 537, 550-600, 600-650, 650-700, 700-750, 750-800, 800-850, 850-900, 950-1000, 1100-1200, 1200-1500, 1500-2000, 2000-2500, 2500-3000, 3000-3500 and 3500-3600 nucleotides in length and hybridizes under stringent hybridization conditions to a nucleic acid molecule of SEQ ID NO:12, or the nucleotide sequence of the DNA insert of the plasmid deposited with ATCC as Accession Number ______.

Probes based on the LGR6 nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an LGR6 protein, such as by measuring a level of an LGR6-encoding nucleic acid in a sample of cells from a subject e.g., detecting LGR6 mRNA levels or determining whether a genomic LGR6 gene has been mutated or deleted.

A nucleic acid fragment encoding a “biologically active portion of an LGR6 protein” can be prepared by isolating a portion of the nucleotide sequence of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, which encodes a polypeptide having an LGR6 biological activity (the biological activities of the LGR6 proteins are described herein), expressing the encoded portion of the LGR6 protein (e.g., by recombinant expression in vitro) and assessing the activity of the encoded portion of the LGR6 protein.

For example, a nucleic acid fragment encoding a biologically active portion of LGR6 includes one or more of a leucine-rich repeat, e.g., amino acid residues 67 to 90, 91 to 114, 115 to 138, 139 to 162, 163 to 186, 187 to 210, 211 to 234, 235 to 257, 258 to 281, 282 to 305, 306 to 329, 330 to 352, 353 to 375, 376 to 398, 399 to 422, and 423 to 446 of SEQ ID NO:2; an RGD cell attachment site, e.g., amino acid residues 760-762 of SEQ ID NO:2; a transmembrane domain, e.g., amino acid 566-588, 599-621, 655-674 of SEQ ID NO:2; an N-myristoylation sites from about amino acids 45-50, 99-104, 107-112, 380-385, 398-403, 483-488, 493-498, 513-518, 533-538, 563-568, 602-607, 612-617, 641-646, 652-657, 684-689, 698-703, 886-891, 922-927, 942-947, 949-954 and 960-965 of SEQ ID NO:2; a protein kinase C phosphorylation site for example, from amino acids 19-21, 115-117, 142-144, 163-165, 420-422, 685-687 and 844-846 of SEQ ID NO:2; a casein kinase II phosphorylation site, for example, from amino acids 328331, 707-710, 862-865 of SEQ ID NO:2; a tyrosine kinase phosphorylation site, for example, from amino acid 469-475, of SEQ ID NO:2; an N-glycosylation site, for example, from amino acids 77-80 and 208-211 of SEQ ID NO:2; and a glycoaminoglycan attachment site, for example, from amino acid 638-641, of SEQ ID NO:2.

B. LGR6 Nucleic Acid Variants

The invention further encompasses nucleic acid molecules that differ from the nucleotide sequence shown in SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, due to degeneracy of the genetic code and thus encode the same LGR6 proteins as those encoded by the nucleotide sequence shown in SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______. In another embodiment, an isolated nucleic acid molecule of the invention has a nucleotide sequence encoding a protein having an amino acid sequence shown in SEQ ID NO:2, SEQ ID NO:5, SEQ ID NO:8 or SEQ ID NO:11.

In addition to the LGR6 nucleotide sequences shown in SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, it will be appreciated by those skilled in the art that DNA sequence polymorphisms that lead to changes in the amino acid sequences of the LGR6 proteins may exist within a population (e.g., the human population). Such genetic polymorphism in the LGR6 genes may exist among individuals within a population due to natural allelic variation. As used herein, the terms “gene” and “recombinant gene” refer to nucleic acid molecules which include an open reading frame encoding an LGR6 protein, preferably a mammalian LGR6 protein, and can further include non-coding regulatory sequences, and introns.

Allelic variants of human LGR6 include both functional and non-functional LGR6 proteins. Functional allelic variants are naturally occurring amino acid sequence variants of the human LGR6 protein that maintain the ability to bind an LGR6 ligand and/or modulate any of the LGR6 activities described herein. Functional allelic variants will typically contain only conservative substitution of one or more amino acids of SEQ ID NO:2, SEQ ID NO:5, SEQ ID NO:8, or SEQ ID NO:11, or substitution, deletion or insertion of non-critical residues in non-critical regions of the protein.

Non-functional allelic variants are naturally occurring amino acid sequence variants of the human LGR6 protein that do not have the ability to either bind an LGR6 target, e.g., an enzyme and/or modulate any of the LGR6 activities described herein. Non-functional allelic variants will typically contain a non-conservative substitution, a deletion, or insertion or premature truncation of the amino acid sequence of SEQ ID NO:2, SEQ ID NO:5, SEQ ID NO:8, or SEQ ID NO:11, or a substitution, insertion or deletion in critical residues or critical regions.

The present invention further provides non-human orthologues of the human LGR6 protein. Orthologues of the human LGR6 protein are proteins that are isolated from non-human organisms and possess the same LGR6 target binding and/or modulation of signalling mechanisms of the human LGR6 protein. Orthologues of the human LGR6 protein can readily be identified as comprising an amino acid sequence that is substantially homologous to SEQ ID NO:2, SEQ ID NO:5, SEQ ID NO:8 or SEQ ID NO:11.

Moreover, nucleic acid molecules encoding other LGR6 family members and, thus, which have a nucleotide sequence which differs from the LGR6 sequences of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______ are intended to be within the scope of the invention. For example, another LGR6 cDNA can be identified based on the nucleotide sequence of human LGR6. Moreover, nucleic acid molecules encoding LGR6 proteins from different species, and thus which have a nucleotide sequence which differs from the LGR6 sequences of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, are intended to be within the scope of the invention. For example, a mouse LGR6 cDNA can be identified based on the nucleotide sequence of a human LGR6.

Nucleic acid molecules corresponding to natural allelic variants and homologues of the LGR6 cDNAs of the invention can be isolated based on their homology to the LGR6 nucleic acids disclosed herein using the cDNAs disclosed herein, or a portion thereof, as a hybridization probe according to standard hybridization techniques under stringent hybridization conditions.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1, SEQ ID NO:3, SEQ ID NO:4, SEQ ID NO:6, SEQ ID NO:7, SEQ ID NO: 9, SEQ ID NO:10, SEQ ID NO:12, or the nucleotide sequence of the DNA insert of the plasmid deposited with ATCC as Accession Number ______. In other embodiment, the nucleic acid is at least 30, 50, 100, 150, 200, 250, 300, 307, 350, 400, 450, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3500 or 3600 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50° C., preferably at 55° C., and more preferably at 60° C. or 65° C. Preferably, an isolated nucleic acid molecule of the invention that hybridizes under stringent conditions to the sequence of SEQ ID NO:1, SEQ ID NO:4, SEQ ID NO:7 or SEQ ID NO:10, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the LGR6 sequences that may exist in the population, the skilled artisan will further appreciate that changes can be introduced by mutation into the nucleotide sequences of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, thereby leading to changes in the amino acid sequence of the encoded LGR6 proteins, without altering the functional ability of the LGR6 proteins. For example, nucleotide substitutions leading to amino acid substitutions at “non-essential” amino acid residues can be made in the sequence of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______. A “non-essential” amino acid residue is a residue that can be altered from the wild-type sequence of LGR6 (e.g., the sequence of SEQ ID NO:2, SEQ ID NO:5 or SEQ ID NO:8 or SEQ ID NO:11,) without altering the biological activity, whereas an “essential” amino acid residue is required for biological activity. For example, amino acid residues that are conserved among the LGR6 proteins of the present invention, are predicted to be particularly unamenable to alteration. Furthermore, additional amino acid residues that are conserved between the LGR6 proteins of the present invention and other members of the LGR6 families are not likely to be amenable to alteration.

Accordingly, another aspect of the invention pertains to nucleic acid molecules encoding LGR6 proteins that contain changes in amino acid residues that are not essential for activity. Such LGR6 proteins differ in amino acid sequence from SEQ ID NO:2, SEQ ID NO:5, SEQ ID NO:8, or SEQ ID NO:111, yet retain biological activity. In one embodiment, the isolated nucleic acid molecule comprises a nucleotide sequence encoding a protein, wherein the protein comprises an amino acid sequence at least about 60%, 65%, 70%, 75%, 80%, 85%, 90%, 95% or more homologous to SEQ ID NO:2, SEQ ID NO:5, SEQ ID NO:8 or SEQ ID NO:11.

An isolated nucleic acid molecule encoding an LGR6 protein homologous to the protein of SEQ ID NO:2, SEQ ID NO:5 or SEQ ID NO:8 or SEQ ID NO:11 can be created by introducing one or more nucleotide substitutions, additions or deletions into the nucleotide sequence of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, such that one or more amino acid substitutions, additions or deletions are introduced into the encoded protein. Mutations can be introduced into SEQ ID NO:1, SEQ ID NO:3, SEQ ID NO:4. SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______ by standard techniques, such as site-directed mutagenesis and PCR-mediated mutagenesis.

Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an LGR6 protein is preferably replaced with another amino acid residue from the same side chain family. Alternatively, in another embodiment, mutations can be introduced randomly along all or part of an LGR6 coding sequence, such as by saturation mutagenesis, and the resultant mutants can be screened for LGR6 biological activity to identify mutants that retain activity. Following mutagenesis of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______, the encoded protein can be expressed recombinantly and the activity of the protein can be determined.

In a preferred embodiment, a mutant LGR6 protein can be assayed for the ability to (1) interact with a non-LGR6 protein molecule, e.g., an extracellular signal, (e.g., a glycohormone) or a cell surface receptor, (e.g., an integrin); (2) mobilize an intracellular molecule that participates in a signal transduction pathway (e.g., adenylate cyclase or phosphatidylinositol 4,5-bisphosphate (PIP₂), inositol 1,4,5-triphosphate (IP₃)); (3) modulate cell attachment; (4) modulate neural development and maintenance; (5) modulate thermogenesis in adipocytes, e.g., brown adipocytes, or muscle; (6) modulate endocrine function; and (7) modulate cardiovascular activities

C. Antisense LGR6 Nucleic Acid Molecules

In addition to the nucleic acid molecules encoding LGR6 proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire LGR6 coding strand, or to only a portion thereof. In one embodiment, an antisense nucleic acid molecule is antisense to a “coding region” of the coding strand of a nucleotide sequence encoding LGR6. The term “coding region” refers to the region of the nucleotide sequence comprising codons which are translated into amino acid residues (e.g., the coding region of human LGR6 corresponds to SEQ ID NO:6, SEQ ID NO:9 or SEQ ID NO:12). In another embodiment, the antisense nucleic acid molecule is antisense to a “noncoding region” of the coding strand of a nucleotide sequence encoding LGR6. The term “noncoding region” refers to 5′ and 3′ sequences which flank the coding region that are not translated into amino acids (i.e., also referred to as 5′ and 3′ untranslated regions).

Given the coding strand sequences encoding LGR6 disclosed herein (e.g., SEQ ID NO:3, SEQ ID NO:6, SEQ ID NO:9 or SEQ ID NO:12), antisense nucleic acids of the invention can be designed according to the rules of Watson and Crick base pairing. The antisense nucleic acid molecule can be complementary to the entire coding region of LGR6 mRNA, but more preferably is an oligonucleotide which is antisense to only a portion of the coding or noncoding region of LGR6 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an LGR6 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pot II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue-et al. (1987) FEBS Lett. 215:327-330).

D. LGR6-Specific Ribozymes

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave LGR6 mRNA transcripts to thereby inhibit translation of LGR6 mRNA. A ribozyme having specificity for an LGR6-encoding nucleic acid can be designed based upon the nucleotide sequence of an LGR6 cDNA disclosed herein (i.e., SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the nucleotide sequence of the DNA insert of the plasmid deposited with ATCC as Accession Number ______, ______ or ______). For example, a derivative of a Tetrahymena L-19 IVS RNA can be constructed in which the nucleotide sequence of the active site is complementary to the nucleotide sequence to be cleaved in an LGR6-encoding mRNA. See, e.g., Cech et al. U.S. Pat. No. 4,987,071; and Cech et al. U.S. Pat. No. 5,116,742. Alternatively, LGR6 mRNA can be used to select a catalytic RNA having a specific ribonuclease activity from a pool of RNA molecules. See, e.g., Bartel, D. and Szostak, J. W. (1993) Science 261:1411-1418.

Alternatively, LGR6 gene expression can be inhibited by targeting nucleotide sequences complementary to the regulatory region of the LGR6 (e.g., the LGR6 promoter and/or enhancers) to form triple helical structures that prevent transcription of the LGR6 gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

E. Modified LGR6 Nucleic Acid Molecules

In yet another embodiment, the LGR6 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of LGR6 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of LGR6 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of LGR6 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of LGR6 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous LGR6 gene within a cell line or microorganism may be modified by inserting a heterologous DNA regulatory element into the genome of a stable cell line or cloned microorganism such that the inserted regulatory element is operatively linked with the endogenous LGR6 gene. For example, an endogenous LGR6 gene which is normally “transcriptionally silent”, i.e., a LGR6 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous LGR6 gene may be activated by insertion of a promiscuous regulatory element that works across cell types.

A heterologous regulatory element may be inserted into a stable cell line or cloned microorganism, such that it is operatively linked with an endogenous LGR6 gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated LGR6 Proteins

One aspect of the invention pertains to isolated LGR6 proteins, and biologically active portions thereof, as well as polypeptide fragments suitable for use as immunogens to raise anti-LGR6 antibodies. In one embodiment, native LGR6 proteins can be isolated from cells or tissue sources by an appropriate purification scheme using standard protein purification techniques. In another embodiment, LGR6 proteins are produced by recombinant DNA techniques. Alternative to recombinant expression, an LGR6 protein or polypeptide can be synthesized chemically using standard peptide synthesis techniques.

An “isolated” or “purified” protein or biologically active portion thereof is substantially free of cellular material or other contaminating proteins from the cell or tissue source from which the LGR6 protein is derived, or substantially free from chemical precursors or other chemicals when chemically synthesized. The language “substantially free of cellular material” includes preparations of LGR6 protein in which the protein is separated from cellular components of the cells from which it is isolated or recombinantly produced. In one embodiment, the language “substantially free of cellular material” includes preparations of LGR6 protein having less than about 30% (by dry weight) of non-LGR6 protein (also referred to herein as a “contaminating protein”), more preferably less than about 20% of non-LGR6 protein, still more preferably less than about 10% of non-LGR6 protein, and most preferably less than about 5% non-LGR6 protein. When the LGR6 protein or biologically active portion thereof is recombinantly produced, it is also preferably substantially free of culture medium, culture medium represents less than about 20%, more preferably less than about 10%, and most preferably less than about 5% of the volume of the protein preparation.

The language “substantially free of chemical precursors or other chemicals” includes preparations of LGR6 protein in which the protein is separated from chemical precursors or other chemicals which are involved in the synthesis of the protein. In one embodiment, the language “substantially free of chemical precursors or other chemicals” includes preparations of LGR6 protein having less than about 30% (by dry weight) of chemical precursors or non-LGR6 chemicals, more preferably less than about 20% chemical precursors or non-LGR6 chemicals, still more preferably less than about 10% chemical precursors or non-LGR6 chemicals, and most preferably less than about 5% chemical precursors or non-LGR6 chemicals.

As used herein, a “biologically active portion” of an LGR6 protein includes a fragment of an LGR6 protein which participates in an interaction between an LGR6 molecule and a non-LGR6 molecule. Biologically active portions of an LGR6 protein include peptides comprising amino acid sequences sufficiently homologous to or derived from the amino acid sequence of the LGR6 protein, e.g., the amino acid sequence shown in SEQ ID NO:2, SEQ ID NO:5 SEQ ID NO:8, or SEQ ID NO:11, which include less amino acids than the full length LGR6 proteins, and exhibit at least one activity of an LGR6 protein. Typically, biologically active portions comprise a domain or motif with at least one activity of the LGR6 protein, e.g., regulating reduction of a disulfide bond. A biologically active portion of an LGR6 protein can be a polypeptide which is, for example, 10, 25, 50, 100, 200 or 250 amino acids in length. Biologically active portions of an LGR6 protein can be used as targets for developing agents which modulate an LGR6 protein mediated activity.

In one embodiment, a biologically active portion of an LGR6 protein comprises at least one transmembrane domain. In another embodiment, a biologically active portion of an LGR6 comprises at least one extracellular domain. In yet another embodiment, a biologically active portion of an LGR6 protein comprises at least one leucine-rich repeat. In yet another embodiment a biologically active portion of an LGR6 protein comprises at least one extracellular domain, at least one transmembrane domain and at least one leucine-rich repeat.

It is to be understood that a preferred biologically active portion of an LGR6 protein of the present invention may contain at least one of the above-identified structural domains. A more preferred biologically active portion of an LGR6 protein may contain at least two of the above-identified structural domains. Moreover, other biologically active portions, in which other regions of the protein are deleted, can be prepared by recombinant techniques and evaluated for one or more of the functional activities of a native LGR6 protein.

In a preferred embodiment, the LGR6 protein has an amino acid sequence shown in SEQ ID NO:2, SEQ ID NO:5, SEQ ID NO:8 or SEQ ID NO:11. In other embodiments, the LGR6 protein is substantially homologous to SEQ ID NO:2, SEQ ID NO:5 SEQ ID NO:8 or SEQ ID NO:11, and retains the functional activity of the protein of SEQ ID NO:2, SEQ ID NO:5 SEQ ID NO:8 or SEQ ID NO:11, yet differs in amino acid sequence due to natural allelic variation or mutagenesis, as described in detail in subsection I above. Accordingly, in another embodiment, the LGR6 protein is a protein which comprises an amino acid sequence at least about 60%, 65%, 70%, 75%, 80%, 85%, 90%, 95% or more homologous to SEQ ID NO:2, SEQ ID NO:5 SEQ ID NO:8 or SEQ ID NO:11.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the LGR6 amino acid sequence of SEQ ID NO:2, having 967 amino acid residues, at least 290, preferably at least 387, more preferably at least 484, even more preferably at least 580, and even more preferably at least 680, 774 or 870 amino acid residues are aligned; or, when aligning a second sequence to the LGR6 amino acid sequence of SEQ ID NO:5, having 633 amino acid residues, at least 190, preferably at least 253, more preferably at least 317, even more preferably at least 380, and even more preferably at least 443, 506 or 570 can be aligned). The amino acid residues or nucleotides at corresponding amino acid positions or nucleotide positions are then compared. When a position in the first sequence is occupied by the same amino acid residue or nucleotides the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to LGR6 nucleic acid molecules of the invention. BLAST protein searches can be performed with the XBLAST program, score=50, wordlength=3 to obtain amino acid sequences homologous to LGR6 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

A. LGR6 Chimeric or Fusion Proteins

The invention also provides LGR6 chimeric or fusion proteins. As used herein, an LGR6 “chimeric protein” or “fusion protein” comprises an LGR6 polypeptide operatively linked to a non-LGR6 polypeptide. An “LGR6 polypeptide” refers to a polypeptide having an amino acid sequence corresponding to LGR6, whereas a “non-LGR6 polypeptide” refers to a polypeptide having an amino acid sequence corresponding to a protein which is not substantially homologous to the LGR6 protein, e.g., a protein which is different from the LGR6 protein and which is derived from the same or a different organism. Within an LGR6 fusion protein the LGR6 polypeptide can correspond to all or a portion of an LGR6 protein. In a preferred embodiment, an LGR6 fusion protein comprises at least one biologically active portion of an LGR6 protein. In another preferred embodiment, an LGR6 fusion protein comprises at least two biologically active portions of an LGR6 protein. Within the fusion protein, the term “operatively linked” is intended to indicate that the LGR6 polypeptide and the non-LGR6 polypeptide are fused in-frame to each other. The non-LGR6 polypeptide can be fused to the N-terminus or C-terminus of the LGR6 polypeptide.

For example, in one embodiment, the fusion protein is a GST-LGR6 fusion protein in which the LGR6 sequences are fused to the C-terminus of the GST sequences. Such fusion proteins can facilitate the purification of recombinant LGR6. In another embodiment, the fusion protein is an LGR6 protein containing a heterologous signal sequence at its N-terminus. In certain host cells (e.g., mammalian host cells), expression and/or secretion of LGR6 can be increased through use of a heterologous signal sequence. In yet another embodiment, the fusion protein is a green fluorescent protein (GFP)-LGR6 fusion protein in which the LGR6 sequences are fused to GFP sequences. Such fusion proteins can facilitate the visualization of recombinant LGR6, for example, in cells expressing a GFP-LGR6 fusion protein.

The LGR6 fusion proteins of the invention can be incorporated into pharmaceutical compositions and administered to a subject in vivo. The LGR6 fusion proteins can be used to affect the bioavailability of an LGR6 substrate. Use of LGR6 fusion proteins may be useful therapeutically for the treatment of a disorders, e.g., weight disorders such as obesity, anorexia, cachexia; or a a cardiovascular disorder such as atherosclerosis, ischaemia reperfusion injury, cardiac-hypertrophy, hypertension, coronary artery disease, myocardial infarction, arrythmia, cardiomyopathies, and congestive heart failure.

Moreover, the LGR6-fusion proteins of the invention can be used as immunogens to produce anti-LGR6 antibodies in a subject, to purify LGR6 ligands and in screening assays to identify molecules which inhibit the interaction of LGR6 with an LGR6 substrate.

Preferably, an LGR6 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An LGR6encoding nucleic acid can be cloned into such an expression vector such that the fusion moiety is linked in-frame to the LGR6 protein.

B. Variants of LGR6 Proteins

The present invention also pertains to variants of the LGR6 proteins which function as either LGR6 agonists (mimetics) or as LGR6 antagonists. Variants of the LGR6 proteins can be generated by mutagenesis, e.g., discrete point mutation or truncation of an LGR6 protein. An agonist of the LGR6 proteins can retain substantially the same, or a subset, of the biological activities of the naturally occurring form of an LGR6 protein. An antagonist of an LGR6 protein can inhibit one or more of the activities of the naturally occurring form of the LGR6 protein by, for example, competitively modulating a biological activity of an LGR6 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the LGR6 protein.

In one embodiment, variants of an LGR6 protein which function as either LGR6 agonists (mimetics) or as LGR6 antagonists can be identified by screening combinatorial libraries of mutants, e.g., truncation mutants, of an LGR6 protein for LGR6 protein agonist or antagonist activity. In one embodiment, a variegated library of LGR6 variants is generated by combinatorial mutagenesis at the nucleic acid level and is encoded by a variegated gene library. A variegated library of LGR6 variants can be produced by, for example, enzymatically ligating a mixture of synthetic oligonucleotides into gene sequences such that a degenerate set of potential LGR6 sequences is expressible as individual polypeptides, or alternatively, as a set of larger fusion proteins (e.g., for phage display) containing the set of LGR6 sequences therein. There are a variety of methods which can be used to produce libraries of potential LGR6 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential LGR6 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al (1983) Nucleic Acid Res. 11:477.

In addition, libraries of fragments of an LGR6 protein coding sequence can be used to generate a variegated population of LGR6 fragments for screening and subsequent selection of variants of an LGR6 protein. In one embodiment, a library of coding sequence fragments can be generated by treating a double stranded PCR fragment of an LGR6 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the LGR6 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of LGR6 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify LGR6 variants (Arkin and Yourvan (1992) Proc. Natl. Acad. ScL USA 89:7811-7815; Delgrave et al. (1993) Protein Engineering 6(3):327-331).

In one embodiment, cell based assays can be exploited to analyze a variegated LGR6 library. For example, a library of expression vectors can be transfected into a cell line which ordinarily synthesizes LGR6. The transfected cells are then cultured such that LGR6 and a particular mutant LGR6 are expressed and the effect of expression of the mutant on LGR6 activity in the cells can be detected, e.g., by any of a number of enzymatic assays or by detecting the enzymatic activity. Plasmid DNA can then be recovered from the cells which score for inhibition, or alternatively, potentiation of LGR6 activity, and the individual clones further characterized.

III. Anti-LGR6 Antibodies

An isolated LGR6 protein, or a portion or fragment thereof, can be used as an immunogen to generate antibodies that bind LGR6 using standard techniques for polyclonal and monoclonal antibody preparation. A full-length LGR6 protein can be used or, alternatively, the invention provides antigenic peptide fragments of LGR6 for use as immunogens. The antigenic peptide of LGR6 comprises at least 8 amino acid residues of the amino acid sequence shown in SEQ ID NO:2, SEQ ID NO:5 SEQ ID NO:8 or SEQ ID NO:11 and encompasses an epitope of LGR6 such that an antibody raised against the peptide forms a specific-immune complex with LGR6. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of LGR6 that are located on the surface of the protein, e.g., hydrophilic regions, as well as regions with high antigenicity (see, for example, FIG. 9). For example, an Emini surface probability analysis of the human LGR6 protein sequence can be used to indicate the regions that have a particularly high probability of being localized to the surface of the LGR6 protein and are thus likely to constitute surface residues useful for targeting antibody production.

A LGR6 immunogen typically is used to prepare antibodies by immunizing a suitable subject, (e.g., rabbit, goat, mouse or other mammal) with the immunogen. An appropriate immunogenic preparation can contain, for example, recombinantly expressed LGR6 protein or a chemically synthesized LGR6 polypeptide. The preparation can further include an adjuvant, such as Freund's complete or incomplete adjuvant, or similar immunostimulatory agent. Immunization of a suitable subject with an immunogenic LGR6 preparation induces a polyclonal anti-LGR6 antibody response.

Accordingly, another aspect of the invention pertains to anti-LGR6 antibodies. The term “antibody” as used herein refers to immunoglobulin molecules and immunologically active portions of immunoglobulin molecules, i.e., molecules that contain an antigen binding site which specifically binds (immunoreacts with) an antigen, such as LGR6. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind LGR6. The term “monoclonal antibody” or “monoclonal antibody composition”, as used herein, refers to a population of antibody molecules that contain only one species of an antigen binding site capable of immunoreacting with a particular epitope of LGR6. A monoclonal antibody composition thus typically displays a single binding affinity for a particular LGR6 protein with which it immunoreacts.

Polyclonal anti-LGR6 antibodies can be prepared as described above by immunizing a suitable subject with an LGR6 immunogen. The anti-LGR6 antibody titer in the immunize subject can be monitored over time by standard techniques, such as with an enzyme linked immunosorbent assay (ELISA) using immobilized LGR6. If desired, the antibody molecules directed against LGR6 can be isolated from the mammal (e.g., from the blood) and further purified by well known techniques, such as protein A chromatography to obtain the IgG fraction. At an appropriate time after immunization, e.g., when the anti-LGR6 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an LGR6 immunogen as described above, and the culture supernatants of the resulting hybridoma cells are screened to identify a hybridoma producing a monoclonal antibody that binds LGR6.

Any of the many well known protocols used for fusing lymphocytes and immortalized cell lines can be applied for the purpose of generating an anti-LGR6 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind LGR6, e.g., using a standard ELISA assay.

Alternative to preparing monoclonal antibody-secreting hybridomas, a monoclonal anti-LGR6 antibody can be identified and isolated by screening a recombinant combinatorial immunoglobulin library (e.g., an antibody phage display library) with LGR6 to thereby isolate immunoglobulin library members that bind LGR6. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-LGR6 antibody, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al., (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-LGR6 antibody (e.g., monoclonal antibody) can be used to isolate LGR6 by standard techniques, such as affinity chromatography or immunoprecipitation. An anti-LGR6 antibody can facilitate the purification of natural LGR6 from cells and of recombinantly produced LGR6 expressed in host cells. Moreover, an anti-LGR6 antibody can be used to detect LGR6 protein (e.g., in a cellular lysate or cell supernatant) in order to evaluate the abundance and pattern of expression of the LGR6 protein. Anti-LGR6 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin and aequorin, and examples of suitable radioactive material include ¹²⁵I, ¹³¹I, ³⁵S or ³H.

IV. Recombinant Expression Vectors and Host Cells

Another aspect of the invention pertains to vectors, preferably expression vectors, containing a nucleic acid encoding an LGR6 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain lost cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., LGR6 proteins, mutant forms of LGR6 proteins, fusion proteins, and the like).

The recombinant expression vectors of the invention can be designed for expression of LGR6 proteins in prokaryotic or eukaryotic cells. For example, LGR6 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in LGR6 activity assays, (e.g., direct assays or competitive assays described in detail below), or to generate antibodies specific for LGR6 proteins, for example. In a preferred embodiment, an LGR6 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the LGR6 expression vector is a yeast expression vector. Examples of vectors for expression in yeast S. cerevisae include pYepSec1 (Baldari, et al., (1987) Embo J. 6:229-234), pMFa (Kuijan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, LGR6 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian-Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to LGR6 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an LGR6 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an LGR6 protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an LGR6 protein. Accordingly, the invention further provides methods for producing an LGR6 protein using the host cells of the invention. In one embodiment, the method comprises culturing the host cell of invention (into which a recombinant expression vector encoding an LGR6 protein has been introduced) in a suitable medium such that an LGR6 protein is produced. In another embodiment, the method further comprises isolating an LGR6 protein from the medium or the host cell.

The host cells of the invention can also be used to produce non-human transgenic animals. For example, in one embodiment, a host cell of the invention is a fertilized oocyte or an embryonic stem cell into which LGR6-coding sequences have been introduced. Such host cells can then be used to create non-human transgenic animals in which exogenous LGR6 sequences have been introduced into their genome or homologous recombinant animals in which endogenous LGR6 sequences have been altered. Such animals are useful for studying the function and/or activity of an LGR6 and for identifying and/or evaluating modulators of LGR6 activity. As used herein, a “transgenic animal” is a non-human animal, preferably a mammal, more preferably a rodent such as a rat or mouse, in which one or more of the cells of the animal includes a transgene. Other examples of transgenic animals include non-human primates sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous LGR6 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an LGR6-encoding nucleic acid into the male pronuclei of a fertilized oocyte, e.g. by microinjection, retroviral infection, and allowing the oocyte to develop in a pseudopregnant female foster animal. The LGR6 cDNA sequence of SEQ ID NO:1, SEQ ID NO:4, SEQ ID NO:7 or SEQ ID NO:10 can be introduced as a transgene into the genome of a non-human animal. Alternatively, a nonhuman homologue of a human LGR6 gene, such as a mouse or rat LGR6 gene, can be used as a transgene. Alternatively, an LGR6 gene homologue, such as another LGR6 family member, can be isolated based on hybridization to the LGR6 cDNA sequences of SEQ ID NO:1, SEQ ID NO:3, SEQ ID NO:4, SEQ ID NO:6, SEQ ID NO:7, SEQ ID NO:9, SEQ ID NO:10 or SEQ ID NO:12, or the DNA insert of the plasmid deposited with ATCC as Accession Number ______, ______ or ______ (described further in subsection I above) and used as a transgene. Intronic sequences and polyadenylation signals can also be included in the transgene to increase the efficiency of expression of the transgene. A tissue-specific regulatory sequence(s) can be operably linked to an LGR6 transgene to direct expression of an LGR6 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y. 1986). Similar methods are used for production of other transgenic animals. A transgenic founder animal can be identified based upon the presence of an LGR6 transgene in its genome and/or expression of LGR6 mRNA in tissues or cells of the animals. A transgenic founder animal can then be used to breed additional animals carrying the transgene. Moreover, transgenic animals carrying a transgene encoding an LGR6 protein can further be bred to other transgenic animals carrying other transgenes.

To create a homologous recombinant animal, a vector is prepared which contains at least a portion of an LGR6 gene into which a deletion, addition or substitution has been introduced to thereby alter, e.g., functionally disrupt, the LGR6 gene. The LGR6 gene can be a mouse gene (e.g., the cDNA of SEQ ID NO:3) or a human gene (e.g., the cDNA of SEQ ID NO:6 or SEQ ID NO:9 or SEQ ID NO:10), but more preferably, is a non-human homologue of a human LGR6 gene (e.g., a cDNA isolated by stringent hybridization with the nucleotide sequence of SEQ ID NO:4 or SEQ ID NO:7). For example, a mouse LGR6 gene can be used to construct a homologous recombination vector suitable for altering an endogenous LGR6 gene in the mouse genome. In a preferred embodiment, the vector is designed such that, upon homologous recombination, the endogenous LGR6 gene is functionally disrupted (i.e., no longer encodes a functional protein; also referred to as a “knock out” vector). Alternatively, the vector can be designed such that, upon homologous recombination, the endogenous LGR6 gene is mutated or otherwise altered but still encodes functional protein (e.g., the upstream regulatory region can be altered to thereby alter the expression of the endogenous LGR6 protein). In the homologous recombination vector, the altered portion of the LGR6 gene is flanked at its 5′ and 3′ ends by additional nucleic acid sequence of the LGR6 gene to allow for homologous recombination to occur between the exogenous LGR6 gene carried by the vector and an endogenous LGR6 gene in an embryonic stem cell. The additional flanking LGR6 nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced LGR6 gene has homologously recombined with the endogenous LGR6 gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad Sci USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The recontructed oocyte is then cultured such that it develops to morula or blastocyte and then transferred to pseudopregnant female foster animal. The offspring borne of this female foster animal will be a clone of the animal from which the cell, e.g., the somatic cell, is isolated.

V. Pharmaceutical Compositions

The LGR6 nucleic acid molecules, fragments of LGR6 proteins, and anti-LGR6 antibodies (also referred to herein as “active compounds”) of the invention can be incorporated in pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of an LGR6 protein or an anti-LGR6 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. Depending on the type and severity of the disease, about 1 μg/kg to 15 mg/kg (e.g., 0.1 to 20 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy can be monitored by standard techniques and assays. An exemplary dosing regimen is disclosed in WO 94/04188. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e. an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

VI. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, an LGR6 protein of the invention has one or more of the following activities: (1) it can interact with (e.g., bind to) an extracellular signal, e.g., a glycohormone, or a cell surface receptor; (2) it can mobilize an intracellular molecule that participates in a signal transduction pathway such as adenylate cyclase or phosphatidylinositol 4,5-bisphosphate (PIP₂), inositol 1,4,5-triphosphate (IP₃); (3) it can modulate cell attachment; (4) it can modulate neural development and maintenance; (5) it can modulate thermogenesis in adipocytes, e.g., brown adipocytes or muscle; (6) modulate endocrine function; or (7) it can modulate cardiovascular activities.

The isolated nucleic acid molecules of the invention can be used, for example, to express LGR6 protein (e.g., via a recombinant expression vector in a host cell in gene therapy applications), to detect LGR6 mRNA (e.g., in a biological sample) or a genetic alteration in an LGR6 gene, and to modulate LGR6 activity, as described further below. The LGR6 proteins can be used to treat disorders characterized by insufficient or excessive production of an LGR6 substrate or production of LGR6 inhibitors. In addition, the LGR6 proteins can be used to screen for naturally occurring LGR6 substrates, to screen for drugs or compounds which modulate LGR6 activity, as well as to treat disorders characterized by insufficient or excessive production of LGR6 protein or production of LGR6 protein forms which have decreased or aberrant activity compared to LGR6 wild type protein (e.g., weight disorder, e.g., obesity, anorexia, cachexia; a cardiovascular disorder, e.g., atherosclerosis, ischaemia reperfusion injury, cardiac hypertrophy, hypertension, coronary artery disease, myocardial infarction, arrythmia, cardiomyopathies, and congestive heart failure; a neural disorder). Moreover, the anti-LGR6 antibodies of the invention can be used to detect and isolate LGR6 proteins, regulate the bioavailability of LGR6 proteins, and modulate LGR6 activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to LGR6 proteins, have a stimulatory or inhibitory effect on, for example, LGR6 expression or LGR6 activity, or have a stimulatory or inhibitory effect on, for example, the expression or activity of LGR6 substrate.

In one embodiment, the invention provides assays for screening candidate or test compounds which are substrates of an LGR6 protein or polypeptide or biologically active portion thereof. In another embodiment, the invention provides assays for screening candidate or test compounds which bind to or modulate the activity of an LGR6 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:206.1; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990)Proc. Natl. Acad. Sci. 87:6378-6382); (Felici (1991)J. Mol. Biol. 222:301-310); (Ladner supra.).

In one embodiment, an assay is a cell-based assay in which a cell which expresses an LGR6 protein or biologically active portion thereof is contacted with a test compound and the ability of the test compound to modulate LGR6 activity is determined. Determining the ability of the test compound to modulate LGR6 activity can be accomplished by monitoring, for example, the release of a neurotransmitter from a cell which expresses LGR6. The cell, for example, can be of mammalian origin. Determining the ability of the test compound to modulate the ability of LGR6 to bind to a substrate can be accomplished, for example, by coupling the LGR6 substrate with a radioisotope or enzymatic label such that binding of the LGR6 substrate to LGR6 can be determined by detecting the labeled LGR6 substrate in a complex. For example, compounds (e.g., LGR6 substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., LGR6 substrate) to interact with LGR6 without the labeling of any of the interactants. For example, a microphysiometer can be used to detect the interaction of a compound with LGR6 without the labeling of either the compound or the LGR6. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and LGR6.

In another embodiment, an assay is a cell-based assay comprising contacting a cell expressing an LGR6 target molecule (e.g., an LGR6 substrate) with a test compound and determining the ability of the test compound to modulate (e.g. stimulate or inhibit) the activity of the LGR6 target molecule. Determining the ability of the test compound to modulate the activity of an LGR6 target molecule can be accomplished, for example, by determining the ability of the LGR6 protein to bind to or interact with the LGR6 target molecule.

Determining the ability of the LGR6 protein or a biologically active fragment thereof, to bind to or interact with an LGR6 target molecule can be accomplished by one of the methods described above for determining direct binding. In a preferred embodiment, determining the ability of the LGR6 protein to bind to or interact with an LGR6 target molecule can be accomplished by determining the activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a target-regulated cellular response.

In yet another embodiment, an assay of the present invention is a cell-free assay in which an LGR6 protein or biologically active portion thereof is contacted with a test compound and the ability of the test compound to bind to the LGR6 protein or biologically active portion thereof is determined. Preferred biologically active portions of the LGR6 proteins to be used in assays of the present invention include fragments which participate in interactions with non-LGR6 molecules, e.g., extracellular ligand, or fragments with high surface probability scores. Binding of the test compound to the LGR6 protein can be determined either directly or indirectly as described above. In a preferred embodiment, the assay includes contacting the LGR6 protein or biologically active portion thereof with a known compound which binds LGR6 to form an assay mixture, contacting the assay mixture with a test compound, and determining the ability of the test compound to interact with an LGR6 protein, wherein determining the ability of the test compound to interact with an LGR6 protein comprises determining the ability of the test compound to preferentially bind to LGR6 or biologically active portion thereof as compared to the known compound.

In another embodiment, the assay is a cell free assay in which an LGR6 protein or biologically active portion thereof is contacted with a test compound and the ability of the test compound to modulate (e.g., stimulate or inhibit) the activity of the LGR6 protein or biologically active portion thereof is determined. Determining the ability of the test compound to modulate the activity of an LGR6 protein can be accomplished, for example, by determining the ability of the LGR6 protein to bind to an LGR6 target molecule by one of the methods described above for determining direct binding. Determining the ability of the LGR6 protein to bind to an LGR6 target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of an LGR6 protein can be accomplished by determining the ability of the LGR6 protein to further modulate the activity of a downstream effector of an LGR6 target molecule. For example, the activity of the effector molecule on an appropriate target can be determined or the binding of the effector to an appropriate target can be determined as previously described.

In yet another embodiment, the cell-free assay involves contacting an LGR6 protein or biologically active portion thereof with a known compound which binds the LGR6 protein to form an assay mixture, contacting the assay mixture with a test compound, and determining the ability of the test compound to interact with the LGR6 protein, wherein determining the ability of the test compound to interact with the LGR6 protein comprises determining the ability of the LGR6 protein to preferentially bind to or modulate the activity of an LGR6 target molecule.

The cell-free assays of the present invention are amenable to use of both soluble and/or membrane-bound forms of isolated proteins (e.g., LGR6 proteins or biologically active portions thereof). In the case of cell-free assays in which a membrane-bound form an isolated protein is used (e.g., an LGR6 protein) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either LGR6 or its target molecule to facilitate separation of complexed from uncomplexed forms of one or both of the proteins, as well as to accommodate automation of the assay. Binding of a test compound to an LGR6 protein, or interaction of an LGR6 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/LGR6 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or LGR6 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of LGR6 binding or activity determined using standard techniques.

Other techniques for immobilizing proteins on matrices can also be used in the screening assays of the invention. For example, either an LGR6 protein or an LGR6 target molecule can be immobilized utilizing conjugation of biotin and streptavidin. Biotinylated LGR6 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with LGR6 protein or target molecules but which do not interfere with binding of the LGR6 protein to its target molecule can be derivatized to the wells of the plate, and unbound target or LGR6 protein trapped in the wells by antibody conjugation. Methods for detecting such complexes, in addition to those described above for the GST-immobilized complexes, include immunodetection of complexes using antibodies reactive with the LGR6 protein or target molecule, as well as enzyme-linked assays which rely on detecting an enzymatic activity associated with the LGR6 protein or target molecule.

In another embodiment, modulators of LGR6 expression are identified in a method wherein a cell is contacted with a candidate compound and the expression of LGR6 mRNA or protein in the cell is determined. The level of expression of LGR6 mRNA or protein in the presence of the candidate compound is compared to the level of expression of LGR6 mRNA or protein in the absence of the candidate compound. The candidate compound can then be identified as a modulator of LGR6 expression based on this comparison. For example, when expression of LGR6 mRNA or protein is greater (statistically significantly greater) in the presence of the candidate compound than in its absence, the candidate compound is identified as a stimulator of LGR6 mRNA or protein expression. Alternatively, when expression of LGR6 mRNA or protein is less (statistically significantly less) in the presence of the candidate compound than in its absence, the candidate compound is identified as an inhibitor of LGR6 mRNA or protein expression. The level of LGR6 mRNA or protein expression in the cells can be determined by methods described herein for detecting LGR6 mRNA or protein.

In yet another aspect of the invention, the LGR6 proteins can be used as “bait proteins” in a two-hybrid assay or three-hybrid assay (see, e.g., U.S. Pat. No. 5,283,317; Zervos et al. (1993) Cell 72:223-232; Madura et al. (1993)J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with LGR6 (“LGR6-binding proteins” or “LGR6-bp”) and are involved in LGR6 activity. Such LGR6-binding proteins are also likely to be involved in the propagation of signals by the LGR6 proteins or LGR6 targets as, for example, downstream elements of an LGR6-mediated signaling pathway (e.g., adenylate cyclase). Alternatively, such LGR6-binding proteins are likely to be LGR6 inhibitors.

The two-hybrid system is based on the modular nature of most transcription factors, which consist of separable DNA-binding and activation domains. Briefly, the assay utilizes two different DNA constructs. In one construct, the gene that codes for an LGR6 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an LGR6-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the LGR6 protein.

This invention further pertains to novel agents identified by the above-described screening assays. Accordingly, it is within the scope of this invention to further use an agent identified as described herein in an appropriate animal model. For example, an agent identified as described herein (e.g., an LGR6 modulating agent, an antisense LGR6 nucleic acid molecule, an LGR6-specific antibody, or an LGR6-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the LGR6 nucleotide sequences, described herein, can be used to map the location of the LGR6 genes on a chromosome. The mapping of the LGR6 sequences to chromosomes is an important first step in correlating these sequences with genes associated with disease.

Briefly, LGR6 genes can be mapped to chromosomes by preparing PCR primers (preferably 15-25 bp in length) from the LGR6 nucleotide sequences. Computer analysis of the LGR6 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the LGR6 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the LGR6 nucleotide sequences to design oligonucleotide primers, sublocalization can be achieved with panels of fragments from specific chromosomes. Other mapping strategies which can similarly be used to map an LGR6 sequence to its chromosome include in situ hybridization-(described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the LGR6 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The LGR6 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the LGR6 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The LGR6 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, SEQ ID NO:4, SEQ ID NO:7, or SEQ ID NO:10 can comfortably provide positive individual identification with a panel of perhaps 10 to 1,000 primers which each yield a noncoding amplified sequence of 100 bases. If predicted coding sequences, such as those in SEQ ID NO:3, SEQ ID NO:6, SEQ ID NO:9, SEQ ID NO:12 are used, a more appropriate number of primers for positive individual identification would be 500-2,000.

If a panel of reagents from LGR6 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of Partial LGR6 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1, SEQ ID NO:4, SEQ ID NO:7 or SEQ ID NO:10 are particularly appropriate for this use as greater numbers of polymorphisms occur in the noncoding regions, making it easier to differentiate individuals using this technique. Examples of polynucleotide reagents include the LGR6 nucleotide sequences or portions thereof, e.g., fragments derived from the noncoding regions of SEQ ID NO:1, SEQ ID NO:4 or SEQ ID NO:7, having a length of at least 20 bases, preferably at least 30 bases.

The LGR6 nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such LGR6 probes can be used to identify tissue by species and/or by organ type.

In a similar fashion, these reagents, e.g., LGR6 primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining LGR6 protein and/or nucleic acid expression as well as LGR6 activity, in the context of a biological sample (e.g., blood, serum, cells, tissue) to thereby determine whether an individual is afflicted with a disease or disorder, or is at risk of developing a disorder, associated with aberrant LGR6 expression or activity. The invention also provides for prognostic (or predictive) assays for determining whether an individual is at risk of developing a disorder associated with LGR6 protein, nucleic acid expression or activity. For example, mutations in an LGR6 gene can be assayed in a biological sample. Such assays can be used for prognostic or predictive purpose to thereby phophylactically treat an individual prior to the onset of a disorder characterized by or associated with LGR6 protein, nucleic acid expression or activity.

Another aspect of the invention pertains to monitoring the influence of agents (e.g., drugs, compounds) on the expression or activity of LGR6 in clinical trials.

These and other agents are described in further detail in the following sections.

1. Diagnostic Assays

An exemplary method for detecting the presence or absence of LGR6 protein or nucleic acid in a biological sample involves obtaining a biological sample from a test subject and contacting the biological sample with a compound or an agent capable of detecting LGR6 protein or nucleic acid (e.g., mRNA, genomic DNA) that encodes LGR6 protein such that the presence of LGR6 protein or nucleic acid is detected in the biological sample. A preferred agent for detecting LGR6 mRNA or genomic DNA is a labeled nucleic acid probe capable of hybridizing to LGR6 mRNA or genomic DNA. The nucleic acid probe can be, for example, a full-length LGR6 nucleic acid, such as the nucleic acid of SEQ ID NO:1, SEQ ID NO:3, SEQ ID NO:4, SEQ ID NO:6, SEQ ID NO:7, SEQ ID NO:9, SEQ ID NO:10, SEQ ID NO:12 or the DNA insert of the plasmid deposited with ATCC as Accession Number ______, ______ or ______, or a portion thereof, such as an oligonucleotide of at least 15, 30, 50, 100, 250 or 500 nucleotides in length and sufficient to specifically hybridize under stringent conditions to LGR6 mRNA or genomic DNA. Other suitable probes for use in the diagnostic assays of the invention are described herein.

A preferred agent for detecting LGR6 protein is an antibody capable of binding to LGR6 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect LGR6 mRNA, protein, or genomic DNA in a biological sample in vitro as well as in vivo. For example, in vitro techniques for detection of LGR6 mRNA include Northern hybridizations and in situ hybridizations. In vitro techniques for detection of LGR6 protein include enzyme linked immunosorbent assays (ELISAs), Western blots, immunoprecipitations and immunofluorescence. In vitro techniques for detection of LGR6 genomic DNA include Southern hybridizations. Furthermore, in vivo techniques for detection of LGR6 protein include introducing into a subject a labeled anti-LGR6 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting LGR6 protein, mRNA, or genomic DNA, such that the presence of LGR6 protein, mRNA or genomic DNA is detected in the biological sample, and comparing the presence of LGR6 protein, mRNA or genomic DNA in the control sample with the presence of LGR6 protein, mRNA or genomic DNA in the test sample.

The invention also encompasses kits for detecting the presence of LGR6 in a biological sample. For example, the kit can comprise a labeled compound or agent capable of detecting LGR6 protein or mRNA in a biological sample; means for determining the amount of LGR6 in the sample; and means for comparing the amount of LGR6 in the sample with a standard. The compound or agent can be packaged in a suitable container. The kit can further comprise instructions for using the kit to detect LGR6 protein or nucleic acid.

2. Prognostic Assays

The diagnostic methods described herein can furthermore be utilized to identify subjects having or at risk of developing a disease or disorder associated with aberrant LGR6 expression or activity. For example, the assays described herein, such as the preceding diagnostic assays or the following assays, can be utilized to identify a subject having or at risk of developing a disorder associated with a misregulationin LGR6 protein activity or nucleic acid expression, such as a weight, cardiovascular neural or endocrine disorder. Alternatively, the prognostic assays can be utilized to identify a subject having or at risk for developing a disorder associated with a misregulation in LGR6 protein activity or nucleic acid expression, such as a weight, cardiovascular, neural or endocrine disorder. Thus, the present invention provides a method for identifying a disease or disorder associated with aberrant LGR6 expression or activity in which a test sample is obtained from a subject and LGR6 protein or nucleic acid (e.g., mRNA or genomic DNA) is detected, wherein the presence of LGR6 protein or nucleic acid is diagnostic for a subject having or at risk of developing a disease or disorder associated with aberrant LGR6 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant LGR6 expression or activity. For example, such methods can be used to determine whether a subject can be effectively treated with an agent for a weight, cardiovascular, neural or endocrine disorder. Thus, the present invention provides methods for determining whether a subject can be effectively treated with an agent for a disorder associated with aberrant LGR6 expression or activity in which a test sample is obtained and LGR6 protein or nucleic acid expression or activity is detected (e.g., wherein the abundance of LGR6 protein or nucleic acid expression or activity is diagnostic for a subject that can be administered the agent to treat a disorder associated with aberrant LGR6 expression or activity).

The methods of the invention can also be used to detect genetic alterations in an LGR6 gene, thereby determining if a subject with the altered gene is at risk for a disorder characterized by misregulation in LGR6 protein activity or nucleic acid expression, such as a weight, cardiovascular, neural or endocrine disorder. In preferred embodiments, the methods include detecting, in a sample of cells from the subject, the presence or absence of a genetic alteration characterized by at least one of an alteration affecting the integrity of a gene encoding an LGR6-protein, or the mis-expression of the LGR6 gene. For example, such genetic alterations can be detected by ascertaining the existence of at least one of 1) a deletion of one or more nucleotides from an LGR6 gene; 2) an addition of one or more nucleotides to an LGR6 gene; 3) a substitution of one or more nucleotides of an LGR6 gene, 4) a chromosomal rearrangement of an LGR6 gene; 5) an alteration in the level of a messenger RNA transcript of an LGR6 gene, 6) aberrant modification of an LGR6 gene, such as of the methylation pattern of the genomic DNA,

7) the presence of a non-wild type splicing pattern of a messenger RNA transcript of an LGR6 gene, 8) a non-wild type level of an LGR6-protein, 9) allelic loss of an LGR6 gene, and 10) inappropriate post-translational modification of an LGR6-protein. As described herein, there are a large number of assays known in the art which can be used for detecting alterations in an LGR6 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci USA 91:360-364), the latter of which can be particularly useful for detecting point mutations in the LGR6-gene (see Abravaya et al. (1995) Nucleic Acids Res 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to an LGR6 gene under conditions such that hybridization and amplification of the LGR6-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an LGR6 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in LGR6 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in LGR6 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the LGR6 gene and detect mutations by comparing the sequence of the sample LGR6 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the LGR6 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type LGR6 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in LGR6 cDNAs obtained from samples of cells. For example, the mutY enzyme of E. coli cleaves A at G/A mismatches and the thymidine DNA glycosylase from HeLa cells cleaves T at G/T mismatches (Hsu et al (1994) Carcinogenesis 15:1657-1662). According to an exemplary embodiment, a probe based on an LGR6 sequence, e.g., a wild-type LGR6 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in LGR6 genes. For example, single strand conformation polymorphism (SSCP) may be used to detect differences in electrophoretic mobility between mutant and wild type nucleic acids (Orita et al. (1989) Proc Natl Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control LGR6 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′-end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an LGR6 gene.

Furthermore, any cell type or tissue in which LGR6 is expressed may be utilized in the prognostic assays described herein.

3. Monitoring of Effects During Clinical Trials

Monitoring the influence of agents (e.g., drugs) on the expression or activity of an LGR6 protein (e.g., the modulation of membrane excitability or resting potential) can be applied not only in basic drug screening, but also in clinical trials. For example, the effectiveness of an agent determined by a screening assay as described herein to increase LGR6 gene expression, protein levels, or upregulate LGR6 activity, can be monitored in clinical trials of subjects exhibiting decreased LGR6 gene expression, protein levels, or downregulated LGR6 activity. Alternatively, the effectiveness of an agent determined by a screening assay to decrease LGR6 gene expression, protein levels, or down-regulate LGR6 activity, can be monitored in clinical trials of subjects exhibiting increased LGR6 gene expression, protein levels, or upregulated LGR6 activity. In such clinical trials, the expression or activity of an LGR6 gene, and preferably, other genes that have been implicated in, for example, an LGR6-associated disorder can be used as a “read out” or markers of the phenotype of a particular cell.

For example, and not by way of limitation, genes, including LGR6, that are modulated in cells by treatment with an agent (e.g., compound, drug or small molecule) which modulates LGR6 activity (e.g., identified in a screening assay as described herein) can be identified. Thus, to study the effect of agents on LGR6-associated disorders, for example, in a clinical trial, cells can be isolated and RNA prepared and analyzed for the levels of expression of LGR6 and other genes implicated in the LGR6-mediated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of LGR6 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of an LGR6 protein, mRNA, or genomic DNA in the preadministration sample; (iii) obtaining one or more post-administration samples from the subject; (iv) detecting the level of expression or activity of the LGR6 protein, mRNA, or genomic DNA in the post-administration samples; (v) comparing the level of expression or activity of the LGR6 protein, mRNA, or genomic DNA in the pre-administration sample with the LGR6 protein, mRNA, or genomic DNA in the post administration sample or samples; and (vi) altering the administration of the agent to the subject accordingly. For example, increased administration of the agent may be desirable to increase the expression or activity of LGR6 to higher levels than detected, i.e., to increase the effectiveness of the agent. Alternatively, decreased administration of the agent may be desirable to decrease expression or activity of LGR6 to lower levels than detected, i.e. to decrease the effectiveness of the agent. According to such an embodiment, LGR6 expression or activity may be used as an indicator of the effectiveness of an agent, even in the absence of an observable phenotypic response.

C. Methods of Treatment:

The present invention provides for both prophylactic and therapeutic methods of treating a subject at risk of (or susceptible to) a disorder or having a disorder associated with aberrant LGR6 expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the LGR6 molecules of the present invention or LGR6 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant LGR6 expression or activity, by administering to the subject an LGR6 or an agent which modulates LGR6 expression or at least one LGR6 activity. Subjects at risk for a disease which is caused or contributed to by aberrant LGR6 expression or activity can be identified by, for example, any or a combination of diagnostic or prognostic assays as described herein. Administration of a prophylactic agent can occur prior to the manifestation of symptoms characteristic of the LGR6 aberrancy, such that a disease or disorder is prevented or, alternatively, delayed in its progression. Depending on the type of LGR6 aberrancy, for example, an LGR6, LGR6 agonist or LGR6 antagonist agent can be used for treating the subject. The appropriate agent can be determined based on screening assays described herein.

2. Therapeutic Methods

Another aspect of the invention pertains to methods of modulating LGR6 expression or activity for therapeutic purposes. Accordingly, in an exemplary embodiment, the modulatory method of the invention involves contacting a cell with an LGR6 or agent that modulates one or more of the activities of LGR6 protein activity associated with the cell. An agent that modulates LGR6 protein activity can be an agent as described herein, such as a nucleic acid or a protein, a naturally-occurring target molecule of an LGR6 protein (e.g., an LGR6 substrate), an LGR6 antibody, an LGR6 agonist or antagonist, a peptidomimetic of an GPCR agonist or antagonist, or other small molecule. In one embodiment, the agent stimulates one or more LGR6 activities. Examples of such stimulatory agents include active LGR6 protein and a nucleic acid molecule encoding LGR6 that has been introduced into the cell. In another embodiment, the agent inhibits one or more LGR6 activities. Examples of such inhibitory agents include antisense LGR6 nucleic acid molecules, anti-LGR6 antibodies, and LGR6 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of an LGR6 protein or nucleic acid molecule. In one embodiment, the method involves administering an agent (e.g., an agent identified by a screening assay described herein), or combination of agents that modulates (e.g., upregulates or down-regulates) LGR6 expression or activity. In another embodiment, the method involves administering an LGR6 protein or nucleic acid molecule as therapy to compensate for reduced or aberrant LGR6 expression or activity.

A preferred embodiment of the present invention involves a method for treatment of an LGR6 associated disease or disorder which includes the step of administering a therapeutically effective amount of an LGR6 antibody to a subject. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg bodily weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays as described herein.

Stimulation of LGR6 activity is desirable in situations in which LGR6 is abnormally downregulated and/or in which increased LGR6 activity is likely to have a beneficial effect. For example, stimulation of LGR6 activity is desirable in situations in which an LGR6 is downregulated and/or in which increased LGR6 activity is likely to have a beneficial effect. Likewise, inhibition of LGR6 activity is desirable in situations in which LGR6 is abnormally upregulated and/or in which decreased LGR6 activity is likely to have a beneficial effect.

3. Pharmacogenomics

The LGR6 molecules of the present invention, as well as agents, or modulators which have a stimulatory or inhibitory effect on LGR6 activity (e.g., LGR6 gene expression) as identified by a screening assay described herein can be administered to individuals to treat (prophylactically or therapeutically) LGR6 associated disorders (e.g., a weight disorder, e.g., obesity, cachexia, anorexia; a cardiovascular disorder, e.g., atherosclerosis, ischaemia reperfusion injury, cardiac hypertrophy, hypertension, coronary artery disease, myocardial infarction, arrythmia, cardiomyopathies, and congestive heart failure; a neural disorder, e.g., a CNS disorder; or an endocrine disorder) associated with aberrant LGR6 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an LGR6 molecule or LGR6 modulator as well as tailoring the dosage and/or therapeutic regimen of treatment with an LGR6 molecule or LGR6 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic, conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., an LGR6 protein of the present invention), all common variants of that gene can be fairly easily identified in the population and it can be determined if having one version of the gene versus another is associated with a particular drug response.

As an illustrative embodiment, the activity of drug metabolizing-enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., an LGR6 molecule or LGR6 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge edge, when applied to dosing or drug selection, can avoid adverse reactions or therapeutic failure and thus enhance therapeutic or prophylactic efficiency when treating a subject with an LGR6 molecule or LGR6 modulator, such as a modulator identified by one of the exemplary screening assays described herein.

This invention is further illustrated by the following examples which should not be construed as limiting. The contents of the figures, the sequence listing, and all references, patents and published patent applications cited throughout this application are incorporated herein by reference.

### EXAMPLES

### Example 1

### Identification And Characterization of LGR6 cDNAs

In this example, the identification and characterization of the cDNAs encoding mouse LGR6 (clone ftmzb048h10) and human LGR6 (clone fahr) are described.

Isolation of the Mouse and Human LGR6 cDNAs

The invention is based, at least in part, on the discovery of a mouse nucleic acid molecule and human nucleic acid molecule encoding novel LGR6 polypeptides, also referred to herein by the clone designation ftmzb048h10 and human fahr, respectively (and collectively referred to as LGR6).

The mouse LGR6 gene (ftmzb048h10) was isolated from a cDNA library which was prepared from mouse brain. Briefly, mRNA was isolated from mouse brain and a cDNA library was prepared therefrom using art known methods (described in, for example, Molecular Cloning A Laboratory Manual, 2nd Ed., ed. by Sambrook, Fritsch and Maniatis (Cold Spring Harbor Laboratory Press: 1989). Using a program which identifies the presence of signal peptides (Nielsen, H. et al. (1997) Protein Engineering 10:1-6), one positive clone was isolated.

The sequence of the entire clone was determined and found to contain a methionine-initiated open reading frame of about 967 amino acids. Signal peptide algorithms predict that mouse LGR6 (ftmzb048h10) contains a signal peptide (about amino acids-1-23 of SEQ ID NO:2). The mature protein is approximately 943 amino acid residues in length (from about amino acid 24 to amino acid 967 of SEQ ID NO:2). The nucleotide sequence-encoding the mouse LGR6 (ftmzb048h10) precursor protein is shown in FIG. 1 and is set forth as SEQ ID NO:1. The full length protein encoded by this nucleic acid comprises about 967 amino acids and has the amino acid sequence shown in FIG. 1 and set forth as SEQ ID NO:2. The coding region (open reading frame) of SEQ ID NO:1 is set forth in SEQ ID NO:3. The clone comprising the entire coding region of human LGR6 was deposited with the American Type Culture Collection (ATCC®), Rockville, Md., on ______, 1999, and assigned Accession No. ______, presently in Manassas, Va.

Based on the mouse ftmzb048h10 sequence, primers were designed and used to screen a human brain library (obtained from Clonetech). Positive human clones were identified. Subsequently, 5′ RACE PCR was used to obtain a partial nucleotide sequence shown in FIG. 4 and set forth as SEQ ID NO:4. The protein encoded by this nucleic acid comprises about 633 amino-acids and has the amino acid sequence shown in FIG. 5 and set forth as SEQ ID NO:5. The coding region (open reading frame) of SEQ ID NO:4 is set forth in SEQ ID NO:6. Further DNA sequence analysis of the human fahr clone was used to identify additional nucleotide sequences encoding LGR6, as shown in FIG. 8 and set forth as SEQ ID NO:7. The protein encoded by this nucleic acid comprises about 736 amino acids and has the amino acid sequence shown in FIG. 8 and set forth as SEQ ID NO:8. The coding region (open reading frame) of

SEQ ID NO:7 is set forth in SEQ ID NO:9. The clone comprising the entire coding region of human LGR6 was deposited with the American Type Culture Collection (ATCC®), Rockville, Md., on ______, 1999, and assigned Accession No. ______, presently in Manassas, Va.

Further DNA sequence analysis of the human fahr clone was used to identify the full length nucleotide sequences encoding human LGR6, as shown in FIG. 14 and set forth as SEQ ID NO:10. The protein encoded by this nucleic acid comprises about 967 amino acids and has the amino acid sequence shown in FIG. 15 and set forth as SEQ ID NO:11. The coding region (open reading frame) of SEQ ID NO:10 is set forth in SEQ ID NO:12. The clone comprising the entire coding region of human LGR6 was deposited with the American Type Culture Collection (ATCC®), Rockville, Md., on ______, 1999, and assigned Accession No. ______, presently in Manassas, Va.

Analysis of Mouse LGR6 (ftmzb048h10) Nucleic Acid and Protein

A BLASTP 1.4.9 MP-WashU search, using a score of 100 and a word length of 3 (Gish, W. and D. J. States (1993) Nat. Genet. 3:266-272; Altschul et al. (1990) J. Mol. Biol. 215:403) of the amino acid sequence of mouse LGR6 revealed that LGR6 shares some similarity with the following G-protein coupled receptors: Human HG38 (Accession No. AF062006, Genbank Accession Number 424098) (McDonald, T. et al. (1998) Biochem. and Biophys. Res. Comm. 247: 266-270), and rat LGR5 (Accession No. AF061444) and LGR4 (Accession No. AF061443) (Hsu, S. Y. et al. (1998) Mol. Endo. 12 (12): 1830-1845).

The amino acid sequences of human HG38 and rat LGR5 are almost identical except for two amino acids in the N-terminal domain. The percentages of local identity between mouse LGR6 and HG38 revealed 65%, 61% and 59% identity over translated nucleotides 357-1718, 1824-1988 and 2388-2735, respectively, of SEQ ID NO:1. The percentages of local identity were estimated using the BLASTP program. At the amino acid level, LGR6 is about 65% identical to LGR5 at the ligand binding domain (approximately first 560 amino acids) and 49% identical at the 7^{th} transmembrane domain. Therefore, the LGR6 and LGR5 proteins are likely to share the same ligand. In addition, the LGR family (LGR6, LGR5 and LGR4) are structurally related to the glycoprotein receptor family including the receptors for LH, FSH and TSH. These molecules share a large N-terminal extracellular (ecto-) domain containing leucine-rich repeats which are believed to be important for mediating interactions with glycoprotein ligands. The ectodomain of LGR6 contains sixteen leucine-rich repeats compared to nine repeats found in known glycoprotein hormone receptors. LGR6 shares an overall identity of 35% with the FSH, TSH and LH receptors.

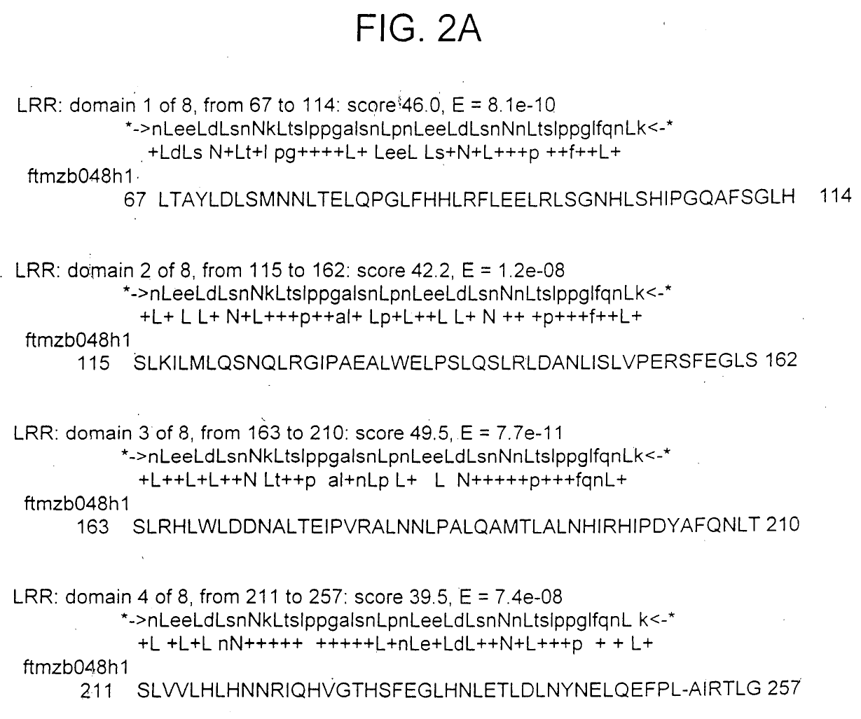

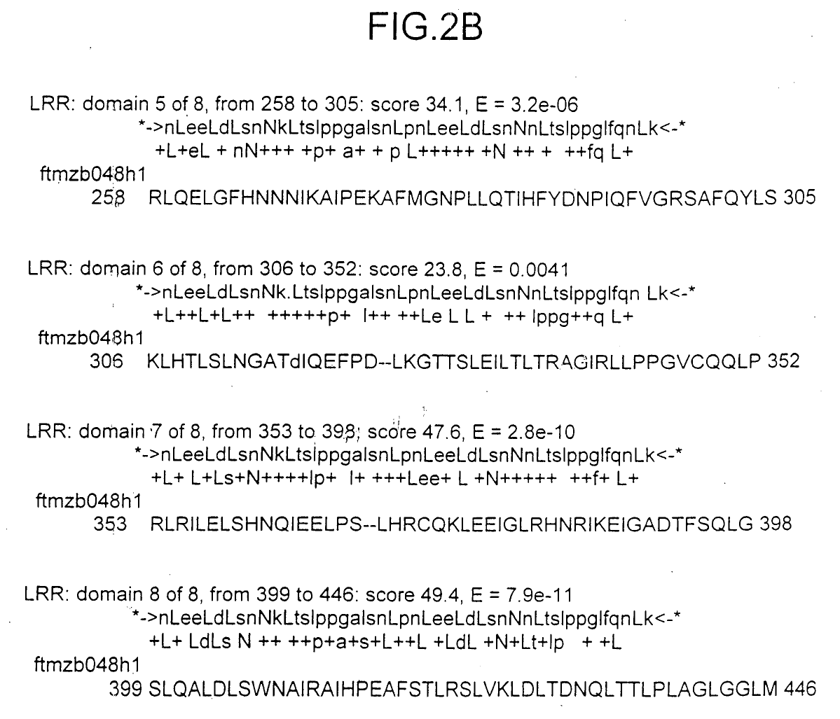

In addition, a Hidden Markov Model (“HMM”) search (HMMER 2.1) of the amino acid sequence of mouse LGR6 (ftmzb048h10) (SEQ ID NO:2) identified eight repeats ((Accession No. PF00560) with a score of 303.4 (E-value 2.3e-17)), each one containing two leucine-rich repeats of about 22 to 25 amino acids in length for a total of sixteen leucine-rich repeats located at about amino acids 67-90, 91-114, 115-138, 139-162, 163-186, 187-210, 211-234, 235-257, 258-281, 282-305, 306-329, 330-352, 353-375, 376-398, 399-422 and 423-446 of SEQ ID NO:2 (FIG. 2). The ectodomains of LGR4 and LGR5 (almost identical to HG38) receptors contain 17 leucine-rich repeats together with N- and C-terminal flanking cysteine-rich sequences, compared with 9 repeats found in known glycoprotein hormone receptors (Hsu, S. Y. et al. (1998) supra).

Mouse LGR6 is further predicted to contain the following domains: one long extracellular domain located at about amino acid residues 1-563 of SEQ ID NO:2; one RGD cell attachment site is located at about amino acid residues 760-762 of SEQ ID NO:2; seven transmembrane domains which extend from about amino acid 564 (extracellular end) to about amino acid 590 (cytoplasmic end) of SEQ ID NO:2; from about amino acid 598 (cytoplasmic end) to about amino acid 620 (extracellular end) of SEQ ID NO:2; from about amino acid 645 (extracellular end) to about amino acid 669 (cytoplasmic end) of SEQ ID NO:2; from about amino acid 684 (cytoplasmic end) to about amino acid 704 (extracellular); from about amino acid 731 (extracellular end) to about amino acid 751 (cytoplasmic end); from about amino acid 773 (cytoplasmic end) to about amino acid 798 (extracellular end); from about amino acid 812 (extracellular end) to about amino acid 834 (cytoplasmic end); three cytoplasmic loops formed at about amino acids 591-597, 670-683, and 752-772 of SEQ ID NO:2; three extracellular loops found at about amino acid 621-644, 705-730 and 799-811 of SEQ ID NO:2; and a C-terminal cytoplasmic domain is found at about amino acid residues 835 to 968 of SEQ ID NO:2.

The mouse LGR6 protein additionally contains seven predicted protein kinase C phosphorylation sites (PS00005) from amino acids 19-21, 115-117, 142-144, 163-165, 420-422, 685-687 and 844-846 of SEQ ID NO:2; five casein kinase II phosphorylation sites (PS00006) from amino acids acids 328-331, 707-710, 862-865, 874-877 and 910-913 of SEQ ID NO:2; one tyrosine kinase phosphorylation site (PS00007) from amino acid 469-475 of SEQ ID NO:2; twenty-one N-myristoylation sites (PS00008) from amino acids 45-50, 99-104, 107-112, 380-385, 398-403, 483-488, 493-498, 513-518, 533-538, 563-568, 602-607, 612-617, 641-646, 652-657, 684-689, 698-703, 886-891, 922-927, 942-947, 949-954 and 960-965 of SEQ ID NO:2; two N-glycosylation sites from about amino acids 77-80 and 208-211 of SEQ ID NO:2; and one glycosaminoglycan attachment site from about amino acids 638-641 of SEQ ID NO:2.

A BLASTN 1.4.9 MP-WashU search, using a score of 100 and a word length of 12 (Altschul et al. (1990) J. Mol. Biol. 215:403) of the nucleotide sequence of mouse LGR6 (ftmzb048h10) revealed local sequence identity in the range of 63-66% between the mouse LGR6 (ftmzb048h10) nucleotide sequence and the nucleotide sequences in HG38 and LGR5 over nucleotides 348-1708, 1848-1981, 2306-2379 and 2399-2734 of SEQ ID NO:1.

Analysis of human LGR6(Fbh150881) Nucleic Acid and Protein

A local alignment of the amino acid sequence of mouse LGR6 (ftmzb048h10) and human LGR6 (Fbh150881) revealed significant identity between the mouse and the human sequences. For example, a local alignment of mouse LGR6 protein with the human LGR6 protein using the the GAP program in the GCG software package, using a Blossum 62 matrix and a gap weight of 12 and a length weight of 4, showed a 89.855% identity between SEQ ID NO:2 (mouse LGR6) and SEQ ID NO:11 (human LGR6) (see FIG. 16).

A Hidden Markov Model (“HMM”) search (HMMER 2.1) of the amino acid sequence of human LGR6 (15088) (SEQ ID NO:11) identified amino acids residues 67 to 90, 91 to 114, 115 to 138, 139 to 162, 163 to 186, 187 to 210, 211 to 234, 235 to 257, 258 to 281, 282 to 305, 306 to 329, 330 to 352, 353 to 375, 376 to 398, 399 to 422 and 423 to 446 of SEQ ID NO:11 as matching the HMM for leucine-rich repeats (Accession No. PF00560). (see FIG. 15).

The amino acid sequence of human LGR6 was analyzed using the program PSORT (http://www. psort.nibb.acjp) to predict the localization of the proteins within the cell. This program assesses the presence of different targeting and localization amino acid sequences within the query sequence. The results of the analyses show that human LGR6 (SEQ ID NO:11) may be localized to the endoplasmic reticulum, to the mitochondrian, to the Golgi, or to secretory vesicles. The results of the analyses further show that human LGR6 (SEQ ID NO:11) also includes an amino-terminal hydrophobic amino acid sequence, consistent with a signal sequence, of about 25 amino acids (from amino acid 1 to about amino acid 25 of SEQ ID NO:11), which upon protease removal results in the production of the mature protein. The mature protein is approximately 943 amino acid residues in length (from about amino acid 25 to amino acid 968 of SEQ ID NO:11).

The human LGR6 (15088) additionally contains one RGD cell attachment site which is located at about amino acid residues 760-762 of SEQ ID NO:11; six transmembrane domains which extend from about amino acid 566 (extracellular end) to about amino acid 590 (cytoplasmic end) of SEQ ID NO:11; from about amino acid 599 (cytoplasmic end) to about amino acid 621 (extracellular end) of SEQ ID NO:11; from about amino acid 646 (extracellular end) to about amino acid 665 (cytoplasmic end) of SEQ ID NO:11; from about amino acid 688 (cytoplasmic end) to about amino acid 709 (extracellular end) of SEQ ID NO:11; from about amino acid 728 (extracellular end) to about amino acid 752 (cytoplasmic end) of SEQ ID NO:11; and from about amino acid 777 (cytoplasmic end) to about amino acid 801 (extracellular end) of SEQ ID NO:11. (see FIG. 15).

The human LGR6 protein (clone 15088) additionally contains six predicted protein kinase C phosphorylation sites (PS00005) from amino acids 19-21, 115-117, 142-144, 163-165, 507-509 and 685-687 of SEQ ID NO:11; four casein kinase II phosphorylation sites (PS00006) from amino acids acids 328-331, 707-710, 862-865 and 874-877 of SEQ ID NO:11; two tyrosine kinase phosphorylation sites (PS00007) from amino acid 469475 and 517-523 of SEQ ID NO:2; nineteen N-myristoylation sites (PS00008) from amino acids amino acids 45-50, 99-104, 107-112, 127-132, 380-385, 483-488, 493-498, 563-568, 602-607, 612-617, 641-646, 652-657, 684-689, 698-703, 725-730, 922-927942-947, 948-953 and 960-965 of SEQ ID NO:11; two N-glycosylation sites from about amino acids 77-80 and 208-211 of SEQ ID NO:11; and one glycosaminoglycan attachment site from about amino acids 951-954 of SEQ ID NO:11; three prokaryotic membra lipoprotein lipid attachment sitees from about amino acids 605-615, 663-673 and 894-904; one leucine zipper pattern from about amino acid 57-78; and one C-terminal targeting signal from about amino acid 965-968.

To identify the presence of an aldehyde dehydrogenase oxidoreductase domain in a LGR6 protein, and to make the determination that a protein of interest has a particular profile, the amino acid sequence of the protein is searched against a database of known protein domains (e.g., the ProDom database) using the default parameters (available at http://www.toulouse.inra.fr/prodom.html). A search was performed against the ProDom database resulting in the identification of an aldehyde dehydrogenase oxidoreductase domain in the amino acid sequence of human LGR6 (SEQ ID NO:1). The results of the search show that the human LGR6 protein (SEQ ID NO:11) has one Glycoprotein EGF-like Domain from about amino acids 70-433 of SEQ ID NO:11; a signal glycoprotein precursor domain at about amino acid residues 535 to 571 and also shares homologous domains with LGR4 and LGR5 at about amino acids 105-336 and 591-666.

Analysis of human LGR6 (fahr) Nucleic Acid and Protein

A local alignment of the amino acid sequence of mouse LGR6 (ftmzb048h10) and human LGR6 (fahr) revealed significant identity between the mouse and the human sequences. For example, an 87.9% identity in an amino acid overlap corresponding to amino acids 370 to 967 of ftmzb048h10 (SEQ ID NO:2) and 30 to 636 of human fahr (SEQ ID NO:5) was revealed (FASTA Search, version 2.0u53 July 1996 with a Smith-Waterman score of 2657; Pearson, W. R. and Lipman, D. J. (1988) Proc. Natl. Acad. Sci. USA 85: 2444-2448). In addition, an alignment of the nucleotide sequence, using a Smith-Waterman score of 9593, revealed a 76.9% identity in a 2493 overlap corresponding to nucleotides 1170 to 2485 of mouse ftmzb048h10 (SEQ ID NO:1) and nucleotides 9 to 2486 of human fahr (SEQ ID NO:4).

A local alignment of mouse LGR6 protein with the human LGR6 protein using the the GAP program in the GCG software package, using a Blossum 62 matrix and a gap weight of 12 and a length weight of 4, showed a 89.281% identity between the two sequences in an amino acid overlap corresponding to residues 201 to 968 of ftmzb048h10 (SEQ ID NO:2) and residues 1 to 737 of human fahr (SEQ ID NO:8) (see FIG. 13). Futhermore, a local alignment of the mouse LGR6 nucleic acid sequence with the human LGR6 nucleic acid sequence using the the GAP program in the GCG software package, using a nwsgapdna matrix, a gap weight of 12 and a length weight of 4 showed a 84.211% identity between the two sequences, in an overlap corresponding to nucleotides 901 to 3637 of mouse ftmzb048h10 (SEQ ID NO:1) and nucleotides 1 to 2711 of human fahr (SEQ ID NO:7) (see FIG. 12).

A Hidden Markov Model (“HMM”) search (HMMER 2.1) of the amino acid sequence of human LGR6 (fahr) (SEQ ID NO:5) identified amino acids 64-87 and 88-111 of SEQ ID NO:5 as matching the HMM for leucine-rich repeats (Accession No. PF00560) with a score of 51.0 (E-value 2.6e-11) (FIG. 6). The domain identified corresponds to two consecutive leucine-rich repeats. Leucine rich repeats were also identified at amino acid residues 4-26, 27-50, 51-74, 75-97, 98-121, 122-143, 144-167, 168-191, and 192-215 of SEQ ID NO:8 (see FIGS. 10 and 11).

Human LGR6 (fahr) protein is further predicted to contain the following sites: one RGD cell attachment site is located at about amino acid residues 425-467 of SEQ ID NO:5, and amino acid residues 529-531 of SEQ ID NO:8; seven transmembrane domains which extend from about amino acid 230 (extracellular end) to about amino acid 256 (cytoplasmic end) of SEQ ID NO:5; from about amino acid 264 (cytoplasmic end) to about amino acid 286 (extracellular end) of SEQ ID NO:5; from about amino acid 311 (extracellular end) to about amino acid 336 (cytoplasmic end) of SEQ ID NO:5; from about amino acid 350 (cytoplasmic end) to about amino acid 370 (extracellular end) of SEQ ID NO:5; from about amino acid 397 (extracellular end) to about amino acid 417 (cytoplasmic end) of SEQ ID NO:5; from about amino acid 440 (cytoplasmic end) to about amino acid 464 (extracellular end) of SEQ ID NO:5; from about amino acid 478 (extracellular end) to about amino acid 500 (cytoplasmic end), and from about amino acid 333 (extracellular end) to about amino acid 359 (cytoplasmic end) of SEQ ID NO:8; from about amino acid 367 (cytoplasmic end) to about amino acid 389 (extracellular end) of SEQ ID NO:8; from about amino acid 414 (extracellular end) to about amino acid 439 (cytoplasmic end) of SEQ ID NO:8; from about amino acid 453 (cytoplasmic end) to about amino acid 473 (extracellular end) of SEQ ID NO:8; from about amino acid 500 (extracellular end) to about amino acid 520 (cytoplasmic end) of SEQ ID NO:8; from about amino acid 543 (cytoplasmic end) to about amino acid 567 (extracellular end) of SEQ ID NO:8; and from about amino acid 581 (extracellular end) to about amino acid 603 (cytoplasmic end) of SEQ ID NO:8; three cytoplasmic loops found at about amino acids 257-263, 337-349 and 418-439 of SEQ ID NO:5, and amino acids 360-366, 440-452 and 521-542 of SEQ ID NO:8; three extracellular loops found at about amino acid 287-310, 371-396 and 465-477 of SEQ ID NO:5, and amino acid residues 390-413, 474-499 and 568-580 of SEQ ID NO:8; and a C-terminal cytoplasmic domain is found at about amino acid residues 501 to 633 of SEQ ID NO:5, and amino acid residues 604-736 of SEQ ID NO:8. The human LGR6 protein additionally contains two 7tm_{—}1 domains at about amino acid residues 404-431 and 553-596 of SEQ ID NO:8 (see FIG. 10).

The human LGR6 (fahr) protein additionally contains predicted protein kinase C phosphorylation sites (PS00005) from amino acids 52-54, 172-174 and 350-352 of SEQ ID NO:5, and amino acids 276-278 and 454-456 of SEQ ID NO:8; casein kinase 11 phosphorylation sites (PS00006) from amino acids acids 372-375, 527-530 and 539-542 of SEQ ID NO:5, and amino acids acids 97-100, 476-479, 631-634 and 643-646 of SEQ ID NO:8; tyrosine kinase phosphorylation site (PS00007) from amino acid 134-140 and 182-188 of SEQ ID NO:5, and amino acids 238-244 and 286-292 of SEQ ID NO:8; N-myristoylation sites (PS00008) from amino acids 17-22, 148-153, 158-163, 228-233, 267-272, 277-282, 306-311, 317-322, 349-354, 363-368, 390-395, 587-592, 607-612, 613-618 and 625-630 of SEQ ID NO:5, and amino acids acids 149-154, 252-257, 262-267, 332-337, 371-376, 381-386, 410-415, 421-426, 453-458, 467-472, 494-499, 691-696, 711-716, 717-722 and 729-734 of SEQ ID NO:8; N-glycosylation sites from about amino acids 1-4 and 48-51 of SEQ ID NO:5; and glycosaminoglycan attachment site from about amino acids 616-619 of SEQ ID NO:5, and amino acids 720-723 of SEQ ID NO:8.

A BLASTN 1.4.9 MP-WashU search, using a score of 100 and a word length of 12 (Altschul et al. (1990) J. Mol. Biol. 215:403) of the nucleotide sequence of mouse ftmzb048h10 revealed a local sequence identity of 99% between human fahr nucleotides 1851 to 2327 of SEQ ID NO:4 and the nucleotide sequences 1 to 477 of human cDNA clone ZD96C01 (Accession No. AF088074).

A BLASTN 2.0 MP-WashU search, using a score of 100 and a word length of 12 (Altschul et al. (1990) J. Mol. Biol. 215:403) of the nucleotide sequence of human fahr revealed a local sequence identity of 99% between human fahr nucleotides 2225 to 2701 of SEQ ID NO:7 and the nucleotide sequences 1 to 477 of human cDNA clone ZD96C01 (Accession No. AF088074), and a local sequence identity of 81% between human fahr nucleotides 1665 to 1730 of SEQ ID NO:7 and nucleotide sequences 175 to 240 of human cDNA clone ZD96C01 (Accession No. AF088074).

A BLASTP 2.0 MP-WashU search, using a score of 100 and a word length of 3 Altschul et al. (1990). Mol. Biol. 215:403) of the amino acid sequence of human fahr revealed local sequence identity between human fahr (SEQ ID NO:8) and the human orphan G-protein coupled receptor HG38 (Accession No. AAC28019), the human G protein coupled receptor LGR5 (Accesssion No. AAC77911), the mouse orphan G protein coupled receptor FEX (Accesssion No. AAD14684, and JG0193),

### Example 2

### Tissue Distribution of LGR6 mRNA by Large-Scale Tissue-Specific Library Sequencing and by Northern Blot Hybridization

This Example describes the tissue distribution of LGR6 mRNA.

Northern blot hybridizations with various RNA samples can be performed under standard conditions and washed under stringent conditions, i.e., 0.2×SSC at 65° C. A DNA probe corresponding to all or a portion of the coding region of LGR6 (SEQ ID NO:3 or SEQ ID NO:6) can be used. The DNA is radioactively labeled with ³²P-dCTP using the Prime-It Kit (Stratagene, La Jolla, Calif.) according to the instructions of the supplier. Filters containing mouse mRNA (Clontech, Palo Alto, Calif.) can be probed in ExpressHyb hybridization solution (Clontech) and washed at high stringency according to manufacturer's recommendations.

As an example, the nucleotide sequence for the partial mouse clone aambb001d112 was labeled as described above and used to probe filters containing adult and embryonic mouse mRNA. As shown in FIG. 7, clone aambb001d112 corresponds to a portion of the full length ftmzb048h10 sequence. Expression of this gene was detected in mouse brown fat (with undetectable levels of expression in white fat), with lower levels of expression detected in the mouse heart and the brain. In the developing mouse (embryonic day 17), the ftmzb048h10 gene is expressed in brown fat, smooth muscle of the heart vessel, smooth muscle of the bronchiole, epithelial cell layer of the trachea, mesenchymal cell layer of the tooth, intravertebral disk and developing flat bone of the skull. In the adult mouse brain, this gene is expressed in the hypothalamus (arcuate nucleus and periventricular nucleus), eppendymal cell layer of the third ventricle close to the arcuate nucleus region, the supraoptic nucleus, the cortex, hippocampus, paraventral, paracentral, medio-dorsal and intradorsal thalamic nuclei.

In humans, the distribution of the LGR6 gene was found in decreasing order of abundance in the human heart, brain and skeletal muscle.

### Example 3

### Recombinant Expression of LGR6 in Bacterial Cells

In this example, LGR6 is expressed as a recombinant glutathione-S-transferase (GST) fusion polypeptide in E. coli and the fusion polypeptide is isolated and characterized. Specifically, LGR6 is fused to GST and this fusion polypeptide is expressed in E. coli, e.g., strain PEB199. Expression of the GST-LGR6 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 4

### Expression of Recombinant LGR6 Protein in Mammalian Cells

The C-terminus of mouse LGR6 was tagged at its C-terminal tail with green flourescent protein (GFP) to monitor its localization in living cells. Briefly, PCR primers were used to amplify the C-terminus of mouse LGR6 to remove the stop codon. Subsequently, a full length mouse LGR6 construct was made and cloned into plasmid pEGFP-N2. This construct was transfected into 293 cells. 293 cells stably expressing LGR6 tagged with GFP were seeded onto 5 cm dishes and visualized. The results demonstrated that LGR6-GFP is uniformly distributed in the plasma membrane, in contrast to the cytoplasmic localization of the GFP control protein. These results corroborate that LGR6 is a GPCR which are cell surface signalling molecules.

To express the LGR6 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire LGR6 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the LGR6 DNA sequence is amplified by PCR using two primers. The 5′ primer contains the restriction site of interest followed by approximately twenty nucleotides of the LGR6 coding sequence starting from the initiation codon; the 3′ end sequence contains complementary sequences to the other restriction site of interest, a translation stop codon, the HA tag or FLAG tag and the last 20 nucleotides of the LGR6 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the LGR6 gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the LGR6-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989. The expression of the LGR6 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 50 mM NaCl, 1% NP-40, 0.1% SDS, 0.5% DOC, 50 mM, Tris, pH 7.5). Both the cell lysate and the culture media are precipitated with an HA specific monoclonal antibody. Precipitated polypeptides are then analyzed by SDS-PAGE.

Alternatively, DNA containing the LGR6 coding sequence is cloned directly into the polylinker of the pcDNA/Amp vector using the appropriate restriction sites. The resulting plasmid is transfected into COS cells in the manner described above, and the expression of the LGR6 polypeptide is detected by radiolabelling and immunoprecipitation using an LGR6 specific monoclonal antibody.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An isolated nucleic acid molecule selected from the group consisting of:
a) a nucleic acid molecule comprising a nucleotide sequence which is at least about 60% identical to the nucleotide sequence of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or the cDNA insert of the plasmid deposited with ATCC as Accession Number ______, ______ or ______ a complement thereof;
b) a nucleic acid molecule comprising a fragment of at least 439 nucleotides of the nucleotide sequence of SEQ ID NO:1, SEQ ID NO:3, the cDNA insert of the plasmid deposited with ATCC as Accession Number ______, ______ or ______ a complement thereof;
c) a nucleic acid molecule comprising a fragment of at least 481 nucleotides of the nucleotide sequence of SEQ ID NO:4, SEQ ID NO:6, the cDNA insert of the plasmid deposited with ATCC as Accession Number ______, or a complement thereof;
d) a nucleic acid molecule comprising a fragment of at least 2175 nucleotides of the nucleotide sequence of SEQ ID NO:7, SEQ ID NO:9, the cDNA insert of the plasmid deposited with ATCC as Accession Number ______, or a complement thereof;
e) a nucleic acid molecule comprising a fragment of at least 439 nucleotides of the nucleotide sequence of SEQ ID NO:10, SEQ ID NO:12, the cDNA insert of the plasmid deposited with ATCC as Accession Number ______, or a complement thereof;
f) a nucleic acid molecule which encodes a polypeptide comprising an amino acid sequence of at least about 60% homologous to the amino acid sequence of SEQ ID NO:2, SEQ ID NO:5, SEQ ID NO:8, SEQ ID NO:11, or an amino acid sequence encoded by the cDNA insert of the plasmid deposited with ATCC as Accession Number ______, ______ or ______;
g) a nucleic acid molecule which encodes a fragment of a polypeptide comprising the amino acid sequence of SEQ ID NO:2 or SEQ ID NO:5 or SEQ ID NO:8, or SEQ ID NO:11, wherein the fragment comprises at least 15 contiguous amino acids of SEQ ID NO:2, SEQ ID NO:5, SEQ ID NO:8, SEQ ID NO:11, or the polypeptide encoded by the cDNA insert of the plasmid deposited with ATCC as Accession Number ______, ______ or ______; and
h) a nucleic acid molecule which encodes a naturally occurring allelic variant of a polypeptide comprising the amino acid sequence of SEQ ID NO:2, SEQ ID NO:5, SEQ ID NO:8, SEQ ID NO:11, or an amino acid sequence encoded by the cDNA insert of the plasmid deposited with ATCC as Accession Number ______, ______ or ______, wherein the nucleic acid molecule hybridizes to a nucleic acid molecule comprising SEQ ID NO:1, SEQ ID NO:3, SEQ ID NO:4, SEQ ID NO:6, SEQ ID NO:7, SEQ ID NO:9, SEQ ID NO:10, SEQ ID NO:12, or a complement thereof under stringent conditions.

2. (canceled)

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. (canceled)

7. A non-human mammalian host cell containing the nucleic acid molecule of claim 1.

8. An isolated polypeptide selected from the group consisting of:
a) a fragment of a polypeptide comprising the amino acid sequence of SEQ ID NO:2 or SEQ ID NO:5, SEQ ID NO:8, SEQ ID NO:11, or the polypeptide encoded by the DNA insert of the plasmid deposited with ATCC as Accession Number ______, ______ or ______, wherein the fragment comprises at least 15 contiguous amino acids of SEQ ID NO:2, SEQ ID NO:5, SEQ ID NO:8, SEQ ID NO:11, or the amino acid sequence encoded by the DNA insert of the plasmid deposited with ATCC as Accession Number ______, ______ or ______;
b) a naturally occurring allelic variant of a polypeptide comprising the amino acid sequence of SEQ ID NO:2 or SEQ ID NO:5, SEQ ID NO:8, SEQ ID NO:11, or an amino acid sequence encoded by the cDNA insert of the plasmid deposited with ATCC as Accession Number ______, ______ or ______, wherein the polypeptide is encoded by a nucleic acid molecule which hybridizes to a nucleic acid molecule comprising SEQ ID NO:1, SEQ ID NO:3, SEQ ID NO:4, SEQ ID NO:6, SEQ ID NO:7, SEQ ID NO:9, SEQ ID NO:10, SEQ ID NO:12 or a complement thereof under stringent conditions; and
c) a polypeptide which is encoded by a nucleic acid molecule comprising a nucleotide sequence which is at least 60% identical to a nucleic acid comprising the nucleotide sequence of SEQ ID NO:1, SEQ ID NO:3, SEQ ID NO:4, SEQ ID NO:6, SEQ ID NO:7, SEQ ID NO:9, SEQ ID NO:10, SEQ ID NO:12, or a complement thereof.

9. The isolated polypeptide of claim 8 comprising the amino acid sequence of SEQ ID NO:2, SEQ ID NO:5, SEQ ID NO:8, SEQ ID NO:11, or an amino acid sequence encoded by the cDNA insert of the plasmid deposited with ATCC as Accession Number ______ or ______.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NO:2, SEQ ID NO:5, SEQ ID NO:8, SEQ ID NO:11, or an amino acid sequence encoded by the cDNA insert of the plasmid deposited with ATCC as Accession Number ______ or ______;
b) a fragment of a polypeptide comprising the amino acid sequence of SEQ ID NO:2, SEQ ID NO:5, SEQ ID NO:8, SEQ ID NO:11, or an amino acid sequence encoded by the cDNA insert of the plasmid deposited with ATCC as Accession Number Number ______ ______ or ______, wherein the fragment comprises at least 15 contiguous amino acids of SEQ ID NO:2, SEQ ID NO:5, SEQ ID NO: 8, SEQ ID NO:11, or an amino acid sequence encoded by the cDNA insert of the plasmid deposited with ATCC as Accession Number ______ ______ or ______; and
c) a naturally occurring allelic variant of a polypeptide comprising the amino acid sequence of SEQ ID NO:2, SEQ ID NO:5, SEQ ID NO:8, SEQ ID NO:11, or an amino acid sequence encoded by the cDNA insert of the plasmid deposited with ATCC as Accession Number ______, ______ or ______, wherein the polypeptide is encoded by a nucleic acid molecule which hybridizes to a nucleic acid molecule comprising SEQ ID NO:1, SEQ ID NO:3, SEQ ID NO:4, SEQ ID NO:6, SEQ ID NO:7, SEQ ID NO:9, SEQ ID NO:10, SEQ ID NO: 12, or a complement thereof under stringent conditions;
comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds to a polypeptide of claim 8; and
b) determining whether the compound binds to the polypeptide in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds to a polypeptide of claim 8 comprising:
a) contacting a polypeptide, or a cell expressing a polypeptide of claim 8 with a test compound; and
b) determining whether the polypeptide binds to the test compound.

20. The method of claim 19, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for LGR6-activity.

21. A method for modulating the activity of a polypeptide of claim 8 comprising contacting a polypeptide or a cell expressing a polypeptide of claim 8 with a compound which binds to the polypeptide in a sufficient concentration to modulate the activity of the polypeptide.

22. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.


SEQUENCE LISTING (12 sequences)

SEQ ID NO: 1 - DNA, 3637 bp, Mus musculus
  CDS (222)..(3122)
  misc_feature (3554): n = any nucleotide
gtcgacccacgcgtccgcactcaacaatgcctgcccctctctgactgcaccgtcccgccgccgctgccgccgccgcgcccaagccaagtcgagcgggggcgttgcccaccgacggcacagcccttgggcccgcccgggaccaggaggtgagccgcgcgcgcacagctccgtgcgctcgcccgtctgagcgcccgccaggtgccccgcagcccgccgccgagatgcacagcccgcctgggctcctggcgctgtggctttgcgctgtgctgtgcgcatcggcgcgcgggggcagcgacccccagcctggcccggggcgtcccgcctgcccggctccctgccactgccaggaggacggcatcatgctgtccgctgactgctccgagctcgggctctcagtggtgcctgcggacctggaccccctgacggcttacctagacctcagtatgaacaacctcacggagcttcagccgggtctcttccaccacctgcgcttcctggaggagctgcggctctcagggaaccacctctcacacatcccgggacaggcattctccggcctccacagcctcaaaattctaatgctgcagagcaaccagctccgtgggatcccagcagaggcactatgggagctgcccagcctgcagtcgctgcgcctagatgctaatctcatctccctggtccctgagagaagctttgaggggctctcctccctccgccacctctggctggatgacaatgcactcactgagatccccgtcagagctctcaacaaccttcctgccctacaggccatgaccttggctctcaaccatatccgccacatccctgactatgccttccagaacctcaccagtcttgtggtgctgcatctacataacaaccgcatccagcatgtggggacccacagcttcgaggggctgcacaatctggagacactagacctgaactataatgagctgcaggagttccccttggctatccggaccctgggcaggctgcaggaattgggtttccataacaacaacatcaaggctatcccagagaaagccttcatgggcaaccctctcctgcagacaatacatttttatgacaacccaatccagtttgtgggaaggtcagcattccagtacctgtctaaactgcatacgctatctttgaatggtgccactgatatccaagagttcccagacctcaaaggcaccactagcctggagatcctgaccctgacccgtgcgggcatcagactgctcccaccgggagtgtgccaacagctgcctaggctccgaatcctggagctgtctcataatcagatcgaggagttacccagcctgcacagatgtcagaagctggaggaaattggcctccgacataacaggatcaaggaaattggtgcagataccttcagccagctgggctccttgcaagctttagacctgagttggaatgccatccgtgccatccaccctgaggctttctcaacccttcgatccttggttaagctggacctgactgacaaccagctgaccacactgcccctggctgggctgggaggcctgatgcacctgaagctcaaagggaacttggccctgtctcaggccttctccaaggacagtttcccaaaactgaggatcctggaggtgccctacgcctaccagtgctgtgcctacggcatctgtgccagcttcttcaagacctctgggcagtggcaggccgaggactttcatccagaagaagaggaggcaccaaagaggcccctgggtctccttgctggacaagctgagaaccactatgacctagacctggatgagctccagatggggacagaggactcaaagccaaaccccagtgtccagtgcagccctgttccaggccccttcaagccctgcgagcacctctttgagagctggggcatccgccttgctgtgtgggccatcgtgctgctctccgtactctgtaacgggctggtgctgctgacagtctttgccagcggacccagcccgctgtcccccgtcaagcttgtggtgggtgcgatggcaggcgccaacgccctgacgggcatttcctgtggtctcctggcctctgtggacgccttgacctatggtcagttcgctgagtatggagcccgctgggagagcggtctgggctgccaggctacgggcttcctggctgtcctgggttcagaggcgtcggtgctgctgctcacactggcggccgtgcagtgcagcatctctgtgacctgcgtccgagcctacgggaaggcgccgtcgcctggcagcgtccgcgcaggcgcactgggatgcctggcgctggccgggctggccgcagcactgccgctggcctcggtgggagagtatggcgcctccccactctgcctgccctacgccccacccgagggccggccggccgccctgggcttcgctgtagccctggtgatgatgaactcgctctgcttcctggtggtggccggcgcctacatcaagctctactgtgacctgccacggggtgactttgaggccgtgtgggactgcgccatggtgcgccacgtggcctggctcatctttgcagatggcctcctctactgccccgtggccttcctcagctttgcctccatgctgggcctcttccctgtcacccccgaggctgtcaagtcagtccttctggtggtgctgcctctgcctgcctgcctcaacccactgctctacctgctcttcaaccctcacttccgggatgaccttcggcggctctggccaagccctcggtccccagggcccctagcctacgctgcagccggtgagctggagaagagctcctgcgactccacccaagcgctggtggctttctcagatgtggatcttattctggaagcttctgaggctgggcagcctcctgggctagagacctatggcttcccttcagtgaccctcatctcccgacatcagccgggggccaccaggctggagggaaaccattttatagagtctgatggaaccaagtttgggaacccacaacctcccatgaagggagaactgctgctgaaggcagagggagccactttggcaggctgtggctcttccgtgggtggagccctctggccctctggctctctctttgcctctcacttgtaaatatccctctctgtttgtcctctccccatccaatgatggctgcttataaaagaaagacaactccaactccatagcaagatggccaacacctctgactccattgttctctctccacgacccctaaccaatgagtgcttccaagtcttgctttgtcttggccttcagcttcactttcaccctgggccttctctgtccaatccaatacttctgacagaggcctgggaaatttgcataggagaaaggagaaaagcaaaagacagtgaaggttattgggccctgacagagccatgatcagtaagtgcagagtgatggggaggtctcacagagcatgacactggaagacaactaccaaagacattggagagtctcccctgtgacatatagaatataaaatgtgttctgcgttccattaatcttgacctatgctgngccaaagtgcttcctgttaaaatacactttggaagacattgaaaaaaaaaaaaaaaaaaaaaaaaaaaaaaaagggcggccgc

SEQ ID NO: 2 - protein, 967 aa, Mus musculus
MHSPPGLLALWLCAVLCASARGGSDPQPGPGRPACPAPCHCQEDGIMLSADCSELGLSVVPADLDPLTAYLDLSMNNLTELQPGLFHHLRFLEELRLSGNHLSHIPGQAFSGLHSLKILMLQSNQLRGIPAEALWELPSLQSLRLDANLISLVPERSFEGLSSLRHLWLDDNALTEIPVRALNNLPALQAMTLALNHIRHIPDYAFQNLTSLVVLHLHNNRIQHVGTHSFEGLHNLETLDLNYNELQEFPLAIRTLGRLQELGFHNNNIKAIPEKAFMGNPLLQTIHFYDNPIQFVGRSAFQYLSKLHTLSLNGATDIQEFPDLKGTTSLEILTLTRAGIRLLPPGVCQQLPRLRILELSHNQIEELPSLHRCQKLEEIGLRHNRIKEIGADTFSQLGSLQALDLSWNAIRAIHPEAFSTLRSLVKLDLTDNQLTTLPLAGLGGLMHLKLKGNLALSQAFSKDSFPKLRILEVPYAYQCCAYGICASFFKTSGQWQAEDFHPEEEEAPKRPLGLLAGQAENHYDLDLDELQMGTEDSKPNPSVQCSPVPGPFKPCEHLFESWGIRLAVWAIVLLSVLCNGLVLLTVFASGPSPLSPVKLVVGAMAGANALTGISCGLLASVDALTYGQFAEYGARWESGLGCQATGFLAVLGSEASVLLLTLAAVQCSISVTCVRAYGKAPSPGSVRAGALGCLALAGLAAALPLASVGEYGASPLCLPYAPPEGRPAALGFAVALVMMNSLCFLVVAGAYIKLYCDLPRGDFEAVWDCAMVRHVAWLIFADGLLYCPVAFLSFASMLGLFPVTPEAVKSVLLVVLPLPACLNPLLYLLFNPHFRDDLRRLWPSPRSPGPLAYAAAGELEKSSCDSTQALVAFSDVDLILEASEAGQPPGLETYGFPSVTLISRHQPGATRLEGNHFIESDGTKFGNPQPPMKGELLLKAEGATLAGCGSSVGGALWPSGSLFASHL

SEQ ID NO: 3 - DNA, 2901 bp, Mus musculus
  CDS (1)..(2901)
atgcacagcccgcctgggctcctggcgctgtggctttgcgctgtgctgtgcgcatcggcgcgcgggggcagcgacccccagcctggcccggggcgtcccgcctgcccggctccctgccactgccaggaggacggcatcatgctgtccgctgactgctccgagctcgggctctcagtggtgcctgcggacctggaccccctgacggcttacctagacctcagtatgaacaacctcacggagcttcagccgggtctcttccaccacctgcgcttcctggaggagctgcggctctcagggaaccacctctcacacatcccgggacaggcattctccggcctccacagcctcaaaattctaatgctgcagagcaaccagctccgtgggatcccagcagaggcactatgggagctgcccagcctgcagtcgctgcgcctagatgctaatctcatctccctggtccctgagagaagctttgaggggctctcctccctccgccacctctggctggatgacaatgcactcactgagatccccgtcagagctctcaacaaccttcctgccctacaggccatgaccttggctctcaaccatatccgccacatccctgactatgccttccagaacctcaccagtcttgtggtgctgcatctacataacaaccgcatccagcatgtggggacccacagcttcgaggggctgcacaatctggagacactagacctgaactataatgagctgcaggagttccccttggctatccggaccctgggcaggctgcaggaattgggtttccataacaacaacatcaaggctatcccagagaaagccttcatgggcaaccctctcctgcagacaatacatttttatgacaacccaatccagtttgtgggaaggtcagcattccagtacctgtctaaactgcatacgctatctttgaatggtgccactgatatccaagagttcccagacctcaaaggcaccactagcctggagatcctgaccctgacccgtgcgggcatcagactgctcccaccgggagtgtgccaacagctgcctaggctccgaatcctggagctgtctcataatcagatcgaggagttacccagcctgcacagatgtcagaagctggaggaaattggcctccgacataacaggatcaaggaaattggtgcagataccttcagccagctgggctccttgcaagctttagacctgagttggaatgccatccgtgccatccaccctgaggctttctcaacccttcgatccttggttaagctggacctgactgacaaccagctgaccacactgcccctggctgggctgggaggcctgatgcacctgaagctcaaagggaacttggccctgtctcaggccttctccaaggacagtttcccaaaactgaggatcctggaggtgccctacgcctaccagtgctgtgcctacggcatctgtgccagcttcttcaagacctctgggcagtggcaggccgaggactttcatccagaagaagaggaggcaccaaagaggcccctgggtctccttgctggacaagctgagaaccactatgacctagacctggatgagctccagatggggacagaggactcaaagccaaaccccagtgtccagtgcagccctgttccaggccccttcaagccctgcgagcacctctttgagagctggggcatccgccttgctgtgtgggccatcgtgctgctctccgtactctgtaacgggctggtgctgctgacagtctttgccagcggacccagcccgctgtcccccgtcaagcttgtggtgggtgcgatggcaggcgccaacgccctgacgggcatttcctgtggtctcctggcctctgtggacgccttgacctatggtcagttcgctgagtatggagcccgctgggagagcggtctgggctgccaggctacgggcttcctggctgtcctgggttcagaggcgtcggtgctgctgctcacactggcggccgtgcagtgcagcatctctgtgacctgcgtccgagcctacgggaaggcgccgtcgcctggcagcgtccgcgcaggcgcactgggatgcctggcgctggccgggctggccgcagcactgccgctggcctcggtgggagagtatggcgcctccccactctgcctgccctacgccccacccgagggccggccggccgccctgggcttcgctgtagccctggtgatgatgaactcgctctgcttcctggtggtggccggcgcctacatcaagctctactgtgacctgccacggggtgactttgaggccgtgtgggactgcgccatggtgcgccacgtggcctggctcatctttgcagatggcctcctctactgccccgtggccttcctcagctttgcctccatgctgggcctcttccctgtcacccccgaggctgtcaagtcagtccttctggtggtgctgcctctgcctgcctgcctcaacccactgctctacctgctcttcaaccctcacttccgggatgaccttcggcggctctggccaagccctcggtccccagggcccctagcctacgctgcagccggtgagctggagaagagctcctgcgactccacccaagcgctggtggctttctcagatgtggatcttattctggaagcttctgaggctgggcagcctcctgggctagagacctatggcttcccttcagtgaccctcatctcccgacatcagccgggggccaccaggctggagggaaaccattttatagagtctgatggaaccaagtttgggaacccacaacctcccatgaagggagaactgctgctgaaggcagagggagccactttggcaggctgtggctcttccgtgggtggagccctctggccctctggctctctctttgcctctcacttg

SEQ ID NO: 4 - DNA, 2486 bp, Homo sapiens
  CDS (2)..(1900)
  misc_feature (172): n = any nucleotide
taatacgactcactatagggaaagctggtacgcctgcaggtaccggtccggaattcccgggtcgacccacgcgtccgtggagcggagccagggtctgagcctgccggctcatccagcctctcttgctgccctagcggcctccaacacaaccgcatctgggaaattggagctngacaccttcagccagctgagctccctgcaagccctggatcttagctggaacgccatccggtccatccaccctgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggccgtgtgggccatcgtgttgctctccgtgctctgcaatggactggtgctgctgaccgtgttcgctggcgggcctgcccccctgcccccggtcaagtttgtggtaggtgcgattgcaggcgccaacaccttgactggcatttcctgtggccttctagcctcagtcgatgccctgacctttggtcagttctctgagtacggagcccgctgggagacggggctaggctgccgggccactggcttcctggcagtacttgggtcggaggcatcggtgctgctgctcactctggccgcagtgcagtgcagcgtctccgtctcctgtgtccgggcctatgggaagtccccctccctgggcagcgttcgagcaggggtcctaggctgcctggcactggcagggctggccgccgcactgcccctggcctcagtgggagaatacggggcctccccactctgcctgccctacgcgccacctgagggtcagccagcagccctgggcttcaccgtggccctggtgatgatgaactccttctgtttcctggtcgtggccggtgcctacatcaaactgtactgtgacctgccgcggggcgactttgaggccgtgtgggactgcgccatggtgaggcacgtggcctggctcatcttcgcagacgggctcctctactgtcccgtggccttcctcagcttcgcctccatgctgggcctcttccctgtcacgcccgaggccgtcaagtctgtcctgctggtggtgctgcccctgcctgcctgcctcaacccactgctgtacctgctcttcaacccccacttccgggatgaccttcggcggcttcggccccgcgcaggggactcagggcccctagcctatgctgcggccggggagctggagaagagctcctgtgattctacccaggccctggtagccttctctgatgtggatctcattctggaagcttctgaagctgggcggccccctgggctggagacctatggcttcccctcagtgaccctcatctcctgtcagcagccaggggcccccaggctggagggcagccattgtgtagagccagaggggaaccactttgggaacccccaaccctccatggatggagaactgctgctgagggcagagggatctacgccagcaggtggaggcttgtcagggggtggcggctttcagccctctggcttggcctttgcttcacacgtgtaaatatccctccccattcttctcttcccctctcttccctttcctctctccccctcggtgaatgatggctgcttctaaaacaaatacaaccaaaactcagcagtgtgatctatagcaggatggcccagtacctggctccactgatcacctctctcctgtgaccatcaccaacgggtgcctcttggcctggctttcccttggccttcctcagcttcaccttgatactgggcctcttccttgtcatgtctgaagctgtggaccagagacctggacttttgtctgcttaagggaaatgagggaagtaaagacagtgaaggggtggagggttgatcagggcacagtggacagggagacctcacagagaaaggcctggaaggtgatttcccgtgtgactcatggataggatacaaaatgtgttccatgtaccattaatcttgacatatgccatgcataaagacttcctattaaaataagctttggaagagattaaaaaaaaaaaaaaagggcggccgctctagaggatccaagcttacgtacgcgtgcatgcgacgtcatagctcttctatagtgtcacctaaattcaatt

SEQ ID NO: 5 - protein, 633 aa, Homo sapiens
  VARIANT (57): Xaa = any amino acid
NTTHYRESWYACRYRSGIPGSTHASVERSQGLSLPAHPASLAALAASNTTASGKLEXDTFSQLSSLQALDLSWNAIRSIHPEAFSTLHSLVKLDLTDNQLTTLPLAGLGGLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLAVWAIVLLSVLCNGLVLLTVFAGGPAPLPPVKFVVGAIAGANTLTGISCGLLASVDALTFGQFSEYGARWETGLGCRATGFLAVLGSEASVLLLTLAAVQCSVSVSCVRAYGKSPSLGSVRAGVLGCLALAGLAAALPLASVGEYGASPLCLPYAPPEGQPAALGFTVALVMMNSFCFLVVAGAYIKLYCDLPRGDFEAVWDCAMVRHVAWLIFADGLLYCPVAFLSFASMLGLFPVTPEAVKSVLLVVLPLPACLNPLLYLLFNPHFRDDLRRLRPRAGDSGPLAYAAAGELEKSSCDSTQALVAFSDVDLILEASEAGRPPGLETYGFPSVTLISCQQPGAPRLEGSHCVEPEGNHFGNPQPSMDGELLLRAEGSTPAGGGLSGGGGFQPSGLAFASHV

SEQ ID NO: 6 - DNA, 1899 bp, Homo sapiens
  CDS (1)..(1899)
  misc_feature (171): n = any nucleotide
aatacgactcactatagggaaagctggtacgcctgcaggtaccggtccggaattcccgggtcgacccacgcgtccgtggagcggagccagggtctgagcctgccggctcatccagcctctcttgctgccctagcggcctccaacacaaccgcatctgggaaattggagctngacaccttcagccagctgagctccctgcaagccctggatcttagctggaacgccatccggtccatccaccctgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggccgtgtgggccatcgtgttgctctccgtgctctgcaatggactggtgctgctgaccgtgttcgctggcgggcctgcccccctgcccccggtcaagtttgtggtaggtgcgattgcaggcgccaacaccttgactggcatttcctgtggccttctagcctcagtcgatgccctgacctttggtcagttctctgagtacggagcccgctgggagacggggctaggctgccgggccactggcttcctggcagtacttgggtcggaggcatcggtgctgctgctcactctggccgcagtgcagtgcagcgtctccgtctcctgtgtccgggcctatgggaagtccccctccctgggcagcgttcgagcaggggtcctaggctgcctggcactggcagggctggccgccgcactgcccctggcctcagtgggagaatacggggcctccccactctgcctgccctacgcgccacctgagggtcagccagcagccctgggcttcaccgtggccctggtgatgatgaactccttctgtttcctggtcgtggccggtgcctacatcaaactgtactgtgacctgccgcggggcgactttgaggccgtgtgggactgcgccatggtgaggcacgtggcctggctcatcttcgcagacgggctcctctactgtcccgtggccttcctcagcttcgcctccatgctgggcctcttccctgtcacgcccgaggccgtcaagtctgtcctgctggtggtgctgcccctgcctgcctgcctcaacccactgctgtacctgctcttcaacccccacttccgggatgaccttcggcggcttcggccccgcgcaggggactcagggcccctagcctatgctgcggccggggagctggagaagagctcctgtgattctacccaggccctggtagccttctctgatgtggatctcattctggaagcttctgaagctgggcggccccctgggctggagacctatggcttcccctcagtgaccctcatctcctgtcagcagccaggggcccccaggctggagggcagccattgtgtagagccagaggggaaccactttgggaacccccaaccctccatggatggagaactgctgctgagggcagagggatctacgccagcaggtggaggcttgtcagggggtggcggctttcagccctctggcttggcctttgcttcacacgtg

SEQ ID NO: 7 - DNA, 2711 bp, Homo sapiens
  CDS (1)..(2208)
gggctgcacaatctggagacactagacctgaattataacaagctgcaggagttccctgtggccatccggaccctgggcagactgcaggaactggggttccataacaacaacatcaaggccatcccagaaaaggccttcatggggaaccctctgctacagacgatacacttttatgataacccaatccagtttgtgggaagatcggcattccagtacctgcctaaactccacacactatctctgaatggtgccatggacatccaggagtttccagatctcaaaggcaccaccagcctggagatcctgaccctgacccgcgcaggcatccggctgctcccatcggggatgtgccaacagctgcccaggctccgagtcctggaactgtctcacaatcaaattgaggagctgcccagcctgcacaggtgtcagaaattggaggaaatcggcctccaacacaaccgcatctgggaaattggagctgacaccttcagccagctgagctccctgcaagccctggatcttagctggaacgccatccggtccatccaccctgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggccgtgtgggccatcgtgttgctctccgtgctctgcaatggactggtgctgctgaccgtgttcgctggcgggcctgcccccctgcccccggtcaagtttgtggtaggtgcgattgcaggcgccaacaccttgactggcatttcctgtggccttctagcctcagtcgatgccctgacctttggtcagttctctgagtacggagcccgctgggagacggggctaggctgccgggccactggcttcctggcagtacttgggtcggaggcatcggtgctgctgctcactctggccgcagtgcagtgcagcgtctccgtctcctgtgtccgggcctatgggaagtccccctccctgggcagcgttcgagcaggggtcctaggctgcctggcactggcagggctggccgccgcactgcccctggcctcagtgggagaatacggggcctccccactctgcctgccctacgcgccacctgagggtcagccagcagccctgggcttcaccgtggccctggtgatgatgaactccttctgtttcctggtcgtggccggtgcctacatcaaactgtactgtgacctgccgcggggcgactttgaggccgtgtgggactgcgccatggtgaggcacgtggcctggctcatcttcgcagacgggctcctctactgtcccgtggccttcctcagcttcgcctccatgctgggcctcttccctgtcacgcccgaggccgtcaagtctgtcctgctggtggtgctgcccctgcctgcctgcctcaacccactgctgtacctgctcttcaacccccacttccgggatgaccttcggcggcttcggccccgcgcaggggactcagggcccctagcctatgctgcggccggggagctggagaagagctcctgtgattctacccaggccctggtagccttctctgatgtggatctcattctggaagcttctgaagctgggcggccccctgggctggagacctatggcttcccctcagtgaccctcatctcctgtcagcagccaggggcccccaggctggagggcagccattgtgtagagccagaggggaaccactttgggaacccccaaccctccatggatggagaactgctgctgagggcagagggatctacgccagcaggtggaggcttgtcagggggtggcggctttcagccctctggcttggcctttgcttcacacgtgtaaatatccctccccattcttctcttcccctctcttccctttcctctctccccctcggtgaatgatggctgcttctaaaacaaatacaaccaaaactcagcagtgtgatctatagcaggatggcccagtacctggctccactgatcacctctctcctgtgaccatcaccaacgggtgcctcttggcctggctttcccttggccttcctcagcttcaccttgatactgggcctcttccttgtcatgtctgaagctgtggaccagagacctggacttttgtctgcttaagggaaatgagggaagtaaagacagtgaaggggtggagggttgatcagggcacagtggacagggagacctcacagagaaaggcctggaaggtgatttcccgtgtgactcatggataggatacaaaatgtgttccatgtaccattaatcttgacatatgccatgcataaagacttcctattaaaataagctttggaagagattaaaaaaaaaaaaaaa

SEQ ID NO: 8 - protein, 736 aa, Homo sapiens
GLHNLETLDLNYNKLQEFPVAIRTLGRLQELGFHNNNIKAIPEKAFMGNPLLQTIHFYDNPIQFVGRSAFQYLPKLHTLSLNGAMDIQEFPDLKGTTSLEILTLTRAGIRLLPSGMCQQLPRLRVLELSHNQIEELPSLHRCQKLEEIGLQHNRIWEIGADTFSQLSSLQALDLSWNAIRSIHPEAFSTLHSLVKLDLTDNQLTTLPLAGLGGLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLAVWAIVLLSVLCNGLVLLTVFAGGPAPLPPVKFVVGAIAGANTLTGISCGLLASVDALTFGQFSEYGARWETGLGCRATGFLAVLGSEASVLLLTLAAVQCSVSVSCVRAYGKSPSLGSVRAGVLGCLALAGLAAALPLASVGEYGASPLCLPYAPPEGQPAALGFTVALVMMNSFCFLVVAGAYIKLYCDLPRGDFEAVWDCAMVRHVAWLIFADGLLYCPVAFLSFASMLGLFPVTPEAVKSVLLVVLPLPACLNPLLYLLFNPHFRDDLRRLRPRAGDSGPLAYAAAGELEKSSCDSTQALVAFSDVDLILEASEAGRPPGLETYGFPSVTLISCQQPGAPRLEGSHCVEPEGNHFGNPQPSMDGELLLRAEGSTPAGGGLSGGGGFQPSGLAFASHV

SEQ ID NO: 9 - DNA, 2208 bp, Homo sapiens
  CDS (1)..(2208)
gggctgcacaatctggagacactagacctgaattataacaagctgcaggagttccctgtggccatccggaccctgggcagactgcaggaactggggttccataacaacaacatcaaggccatcccagaaaaggccttcatggggaaccctctgctacagacgatacacttttatgataacccaatccagtttgtgggaagatcggcattccagtacctgcctaaactccacacactatctctgaatggtgccatggacatccaggagtttccagatctcaaaggcaccaccagcctggagatcctgaccctgacccgcgcaggcatccggctgctcccatcggggatgtgccaacagctgcccaggctccgagtcctggaactgtctcacaatcaaattgaggagctgcccagcctgcacaggtgtcagaaattggaggaaatcggcctccaacacaaccgcatctgggaaattggagctgacaccttcagccagctgagctccctgcaagccctggatcttagctggaacgccatccggtccatccaccctgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggccgtgtgggccatcgtgttgctctccgtgctctgcaatggactggtgctgctgaccgtgttcgctggcgggcctgcccccctgcccccggtcaagtttgtggtaggtgcgattgcaggcgccaacaccttgactggcatttcctgtggccttctagcctcagtcgatgccctgacctttggtcagttctctgagtacggagcccgctgggagacggggctaggctgccgggccactggcttcctggcagtacttgggtcggaggcatcggtgctgctgctcactctggccgcagtgcagtgcagcgtctccgtctcctgtgtccgggcctatgggaagtccccctccctgggcagcgttcgagcaggggtcctaggctgcctggcactggcagggctggccgccgcactgcccctggcctcagtgggagaatacggggcctccccactctgcctgccctacgcgccacctgagggtcagccagcagccctgggcttcaccgtggccctggtgatgatgaactccttctgtttcctggtcgtggccggtgcctacatcaaactgtactgtgacctgccgcggggcgactttgaggccgtgtgggactgcgccatggtgaggcacgtggcctggctcatcttcgcagacgggctcctctactgtcccgtggccttcctcagcttcgcctccatgctgggcctcttccctgtcacgcccgaggccgtcaagtctgtcctgctggtggtgctgcccctgcctgcctgcctcaacccactgctgtacctgctcttcaacccccacttccgggatgaccttcggcggcttcggccccgcgcaggggactcagggcccctagcctatgctgcggccggggagctggagaagagctcctgtgattctacccaggccctggtagccttctctgatgtggatctcattctggaagcttctgaagctgggcggccccctgggctggagacctatggcttcccctcagtgaccctcatctcctgtcagcagccaggggcccccaggctggagggcagccattgtgtagagccagaggggaaccactttgggaacccccaaccctccatggatggagaactgctgctgagggcagagggatctacgccagcaggtggaggcttgtcagggggtggcggctttcagccctctggcttggcctttgcttcacacgtg

SEQ ID NO: 10 - DNA, 3492 bp, Homo sapiens
  CDS (104)...(3004)
ccgccsgcggtgcagcccgccgggaccgggaggcggcagctgcggccaccgcgccgtgcgtccgcgcccggccgccaggtgccccagtagcccgaccgccgagatgcccagcccgccggggctccgggcgctatggctttgcgccgcgctgtgcgcttcccggagggccggcggcgccccccagcccggcccggggcccaccgcctgcccggccccctgccactgccaggaggacggcatcatgctgtctgccgactgctctgagctcgggctgtccgccgttccgggggacctggaccccctgacggcttacctggacctcagcatgaacaacctcacagagcttcagcctggcctcttccaccacctgcgcttcttggaggagctgcgtctctctgggaaccatctctcacacatcccaggacaagcattctctggtctctacagcctgaaaatcctgatgctgcagaacaatcagctgggaggaatccccgcagaggcgctgtgggagctgccgagcctgcagtcgctgcgcctagatgccaacctcatctccctggtcccggagaggagctttgaggggctgtcctccctccgccacctctggctggacgacaatgcactcacggagatccctgtcagggccctcaacaacctccctgccctgcaggccatgaccctggccctcaaccgcatcagccacatccccgactacgcgttccagaatctcaccagccttgtggtgctgcatttgcataacaaccgcatccagcatctggggacccacagcttcgaggggctgcacaatctggagacactagacctgaattataacaagctgcaggagttccctgtggccatccggaccctgggcagactgcaggaactggggttccataacaacaacatcaaggccatcccagaaaaggccttcatggggaaccctctgctacagacgatacacttttatgataacccaatccagtttgtgggaagatcggcattccagtacctgcctaaactccacacactatctctgaatggtgccatggacatccaggagtttccagatctcaaaggcaccaccagcctggagatcctgaccctgacccgcgcaggcatccggctgctcccatcggggatgtgccaacagctgcccaggctccgagtcctggaactgtctcacaatcaaattgaggagctgcccagcctgcacaggtgtcagaaattggaggaaatcggcctccaacacaaccgcatctgggaaattggagctgacaccttcagccagctgagctccctgcaagccctggatcttagctggaacgccatccggtccatccaccctgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggccgtgtgggccatcgtgttgctctccgtgctctgcaatggactggtgctgctgaccgtgttcgctggcgggcctgcccccctgcccccggtcaagtttgtggtaggtgcgattgcaggcgccaacaccttgactggcatttcctgtggccttctagcctcagtcgatgccctgacctttggtcagttctctgagtacggagcccgctgggagacggggctaggctgccgggccactggcttcctggcagtacttgggtcggaggcatcggtgctgctgctcactctggccgcagtgcagtgcagcgtctccgtctcctgtgtccgggcctatgggaagtccccctccctgggcagcgttcgagcaggggtcctaggctgcctggcactggcagggctggccgccgcactgcccctggcctcagtgggagaatacggggcctccccactctgcctgccctacgcgccacctgagggtcagccagcagccctgggcttcaccgtggccctggtgatgatgaactccttctgtttcctggtcgtggccggtgcctacatcaaactgtactgtgacctgccgcggggcgactttgaggccgtgtgggactgcgccatggtgaggcacgtggcctggctcatcttcgcagacgggctcctctactgtcccgtggccttcctcagcttcgcctccatgctgggcctcttccctgtcacgcccgaggccgtcaagtctgtcctgctggtggtgctgcccctgcctgcctgcctcaacccactgctgtacctgctcttcaacccccacttccgggatgaccttcggcggcttcggccccgcgcaggggactcagggcccctagcctatgctgcggccggggagctggagaagagctcctgtgattctacccaggccctggtagccttctctgatgtggatctcattctggaagcttctgaagctgggcggccccctgggctggagacctatggcttcccctcagtgaccctcatctcctgtcagcagccaggggcccccaggctggagggcagccattgtgtagagccagaggggaaccactttgggaacccccaaccctccatggatggagaactgctgctgagggcagagggatctacgccagcaggtggaggcttgtcagggggtggcggctttcagccctctggcttggcctttgcttcacacgtgtaaatatccctccccattcttctcttcccctctcttccctttcctctctccccctcggtgaatgatggctgcttctaaaacaaatacaaccaaaactcagcagtgtgatctatagcaggatggcccagtacctggctccactgatcacctctctcctgtgaccatcaccaacgggtgcctcttggcctggctttcccttggccttcctcagcttcaccttgatactgggcctcttccttgtcatgtctgaagctgtggaccaragacctggacttttgtctgcttaagggaaatgagggaagtaaagacagtgaaggggtggagggttgatcagggcacagtggacagggagacctcacaraaaaaggcctggaaggkgatttcccgtgtgactcatggrtaggawacaaaatgtgttccatgtaccattaatcttgacatatgccatgcataaaracttcctattaaaataagctttggragagatt

SEQ ID NO: 11 - protein, 967 aa, Homo sapiens [sequence omitted]

SEQ ID NO: 12 - DNA, 2901 bp, Homo sapiens
  CDS (1)...(2901)
atgcccagcccgccggggctccgggcgctatggctttgcgccgcgctgtgcgcttcccggagggccggcggcgccccccagcccggcccggggcccaccgcctgcccggccccctgccactgccaggaggacggcatcatgctgtctgccgactgctctgagctcgggctgtccgccgttccgggggacctggaccccctgacggcttacctggacctcagcatgaacaacctcacagagcttcagcctggcctcttccaccacctgcgcttcttggaggagctgcgtctctctgggaaccatctctcacacatcccaggacaagcattctctggtctctacagcctgaaaatcctgatgctgcagaacaatcagctgggaggaatccccgcagaggcgctgtgggagctgccgagcctgcagtcgctgcgcctagatgccaacctcatctccctggtcccggagaggagctttgaggggctgtcctccctccgccacctctggctggacgacaatgcactcacggagatccctgtcagggccctcaacaacctccctgccctgcaggccatgaccctggccctcaaccgcatcagccacatccccgactacgcgttccagaatctcaccagccttgtggtgctgcatttgcataacaaccgcatccagcatctggggacccacagcttcgaggggctgcacaatctggagacactagacctgaattataacaagctgcaggagttccctgtggccatccggaccctgggcagactgcaggaactggggttccataacaacaacatcaaggccatcccagaaaaggccttcatggggaaccctctgctacagacgatacacttttatgataacccaatccagtttgtgggaagatcggcattccagtacctgcctaaactccacacactatctctgaatggtgccatggacatccaggagtttccagatctcaaaggcaccaccagcctggagatcctgaccctgacccgcgcaggcatccggctgctcccatcggggatgtgccaacagctgcccaggctccgagtcctggaactgtctcacaatcaaattgaggagctgcccagcctgcacaggtgtcagaaattggaggaaatcggcctccaacacaaccgcatctgggaaattggagctgacaccttcagccagctgagctccctgcaagccctggatcttagctggaacgccatccggtccatccaccctgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggccgtgtgggccatcgtgttgctctccgtgctctgcaatggactggtgctgctgaccgtgttcgctggcgggcctgcccccctgcccccggtcaagtttgtggtaggtgcgattgcaggcgccaacaccttgactggcatttcctgtggccttctagcctcagtcgatgccctgacctttggtcagttctctgagtacggagcccgctgggagacggggctaggctgccgggccactggcttcctggcagtacttgggtcggaggcatcggtgctgctgctcactctggccgcagtgcagtgcagcgtctccgtctcctgtgtccgggcctatgggaagtccccctccctgggcagcgttcgagcaggggtcctaggctgcctggcactggcagggctggccgccgcactgcccctggcctcagtgggagaatacggggcctccccactctgcctgccctacgcgccacctgagggtcagccagcagccctgggcttcaccgtggccctggtgatgatgaactccttctgtttcctggtcgtggccggtgcctacatcaaactgtactgtgacctgccgcggggcgactttgaggccgtgtgggactgcgccatggtgaggcacgtggcctggctcatcttcgcagacgggctcctctactgtcccgtggccttcctcagcttcgcctccatgctgggcctcttccctgtcacgcccgaggccgtcaagtctgtcctgctggtggtgctgcccctgcctgcctgcctcaacccactgctgtacctgctcttcaacccccacttccgggatgaccttcggcggcttcggccccgcgcaggggactcagggcccctagcctatgctgcggccggggagctggagaagagctcctgtgattctacccaggccctggtagccttctctgatgtggatctcattctggaagcttctgaagctgggcggccccctgggctggagacctatggcttcccctcagtgaccctcatctcctgtcagcagccaggggcccccaggctggagggcagccattgtgtagagccagaggggaaccactttgggaacccccaaccctccatggatggagaactgctgctgagggcagagggatctacgccagcaggtggaggcttgtcagggggtggcggctttcagccctctggcttggcctttgcttcacacgtg